(19) United States
(12) Patent Application Publication
(10) Pub. No.: US 2002/0006618 A1
(43) Pub. Date: Jan. 17, 2002

(54) Methods for using 20893, a human protein kinase

(76) Inventors: Katherine M. Galvin, Jamaica Plain, MA (US); Rosana Kapeller-Libermann, Chestnut Hill, MA (US); Nadine S. Weich, Brookline, MA (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 09/780,949

(22) Filed: Feb. 9, 2001

Related U.S. Application Data
(60) Provisional application No. 60/181,690, filed on Feb. 9, 2000.

(51) Int. Cl. C12Q 1/68; C12Q 1/00

(52) U.S. Cl. 435/6; 435/4

(57) ABSTRACT

The present invention relates to methods for using a human protein kinase belonging to the superfamily of mammalian protein kinases. The invention also relates to methods for using polynucleotides encoding the protein kinase. The invention relates to methods using the protein kinase polypeptides and polynucleotides as a target for diagnosis and treatment in protein kinase-mediated or -related disorders. The invention further relates to drug-screening methods using the protein kinase polypeptides and polynucleotides to identify agonists and antagonists for diagnosis and treatment. The invention further encompasses agonists and antagonists based on the protein kinase polypeptides and polynucleotides. The invention further relates to agonists and antagonists identified by drug screening methods with the protein kinase polypeptides and polynucleotides as a target.

This application claims the benefit of U.S. Provisional Application No. 60/181,690, filed Feb. 9, 2000.

### FIELD OF THE INVENTION

The present invention relates to methods for using a human protein kinase. The invention also relates to methods for using polynucleotides encoding the kinase. The invention further relates to methods using the protein kinase polypeptides and polynucleotides as a target for diagnosis and treatment in protein kinase-mediated or -related disorders. The invention further relates to drug-screening methods using the protein kinase polypeptides and polynucleotides to identify agonists and antagonists for diagnosis and treatment. The invention further encompasses agonists and antagonists based on the protein kinase polypeptides and polynucleotides. The invention further relates to agonists and antagonists identified by drug screening methods with the protein kinase polypeptides and polynucleotides as a target.

### BACKGROUND OF THE INVENTION

Phosphate tightly associated with a molecule, e.g., a protein, has been known since the late nineteenth century. Since then, a variety of covalent linkages of phosphate to proteins have been found. The most common involve esterification of phosphate to serine, threonine, and tyrosine with smaller amounts being linked to lysine, arginine, histidine, aspartic acid, glutamic acid, and cysteine. The occurrence of phosphorylated molecules, e.g., proteins, implies the existence of one or more kinases, e.g., protein kinases, capable of phosphorylating various molecules, e.g., amino acid residues on proteins, and also of phosphatases, e.g., protein phosphatases, capable of hydrolyzing various phosphorylated molecules, e.g., phosphorylated amino acid residues on proteins.

Protein kinases play critical roles in the regulation of biochemical and morphological changes associated with cellular growth and division (D'Urso et al. (1990) Science 250:786-791; Birchmeier et al. (1993) Bioessays 15:185-189). They serve as growth factor receptors and signal transducers and have been implicated in cellular transformation and malignancy (Hunter et al (1992) Cell 70:375-387; Posada et al. (1992) Mol. Biol. Cell 3:583-592; Hunter et al. (1994) Cell 79:573-582). For example, protein kinases have been shown to participate in the transmission of signals from growth-factor receptors (Sturgill et al. (1988) Nature 344:715-718; Gomez et al (1991) Nature 353:170-173), control of entry of cells into mitosis (Nurse (1990) Nature 344:503-508; Maller (1991) Curr. Opin. Cell Biol. 3:269-275) and regulation of actin bundling (Husain-Chishti et al. (1988) Nature 334:718-721).

Protein kinases can be divided into different groups based on either amino acid sequence similarity or specificity for either serine/threonine or tyrosine residues. A small number of dual-specificity kinases have also been described. Within the broad classification, kinases can be further subdivided into families whose members share a higher degree of catalytic domain amino acid sequence identity and also have similar biochemical properties. Most protein kinase family members also share structural features outside the kinase domain that reflect their particular cellular roles. These include regulatory domains that control kinase activity or interaction with other proteins (Hanks et al. (1988) Science 241:42-52).

Extracellular-signal-regulated kinases/microtubule-associated protein kinases (Erk\MAPKs) and cyclin-directed kinases (Cdks) represent two large families of serine-threonine kinases (see Songyang et al., (1996) Mol. Cell. Biol. 16: 6486-6493). Both types of kinases function in cell growth, cell division, and cell differentiation, in response to extracellular stimulae. The Erk\MAPK family members are critical participants in intracellular signaling pathways. Upstream activators as well as the Erk\MAPK components are phosphorylated following contact of cells with growth factors or hormones or after cellular stressors, for example, heat, ultraviolet light, and inflammatory cytokines. These kinases transport messages that have been relayed from the plasma membrane to the cytoplasm by upstream kinases into the nucleus where they phosphorylate transcription factors and effect gene transcription modulation (Karin et al., (1995) Curr. Biol. 5: 747-757). Substrates of the Erk\MAPK family include c-fos, c-jun, APF2, and ETS family members Elk1, Sapla, and c-Ets-1 (cited in Brott et al., (1998) Proc. Natl Acad. Sci. USA 95, 963-968).

Cdks regulate transitions between successive stages of the cell cycle. The activity of these molecules is controlled by phosphorylation events and by association with cyclin. Cdk activity is negatively regulated by the association of small inhibitory molecules (Dynlacht, (1997) Nature 389:148-152). Cdk targets include various transcriptional activators such as p110Rb, p107 and transcription factors, such as p53, E2F and RNA polymerase II, as well as various cytoskeletal proteins and cytoplasmic signaling proteins (cited in Brott et al., above).

A protein has been isolated in Drosophilia, designated nemo, which has homology to Erk\MAPKs and Cdks. A mammalian homologue of nemo, designated NLK, has been reported (Brott et al., above). This protein kinase autophosphorylates and localizes to a great extent in the nucleus. This protein showed homology to both families of kinases (Erk\MAPKs and Cdks). It did not possess the characteristic MAPK phosphorylation motif TXY in the conserved kinase domain VIII. It instead exhibited the sequence TQE resembling the THE sequence found in some Cdks.

More recently, it was shown that NLK could down-regulate HMG-domain-containing proteins related to POP-1. The signaling protein Wnt regulates transcription factors containing high-mobility group (HMG) domains to direct decisions on cell fate during animal development. In C. elegans, the HMG-domain-containing repressor POP-1 distinguishes the fate of anterior daughter cells from posterior daughter cells throughout development. Wnt signaling down-regulates POP-1 activity in posterior daughter cells, for example, E. Meneghini et al., (1999) Nature 399: 793-797, show that the genes MOM-4 and LIT-I were also required to down-regulate POP-I not only in E but in other posterior daughter cells. MOM-4 and LIT-1 are homologous to the mammalian components of the mitogen-activated protein kinase (MAPK) pathway of TAK-1 (transforming growth factor beta activated kinase (and NLK) nemo-like kinase, respectively. MOM-4 and TAK-1 bind related proteins that promote their kinase activity.

The authors of the report concluded that a MAPK-related pathway cooperates with Wnt signal transduction to down-regulate POP-1 activity.

In a further report by the same group (Ishitani et al,(1999) Nature 399: 798-802), it was shown that the TAK-1-NLK-MAPK-related pathway antagonizes signaling between beta-catenin and transcription factor TCF. The Wnt-signaling pathway regulates developmental processes through a complex of beta-catenin and the T-cell factor/lymphoid enhancer factor (TCF\LEF) family of high-mobility group transcription factors. Wnt stabilizes beta-catenin which then binds to TCF and activates gene transcription. This signal pathway is conserved in vertebrates, Drosophilia and C. elegans. In C. elegans, MOM-4 and LIT-I regulate Wnt signaling during embryogenesis. MOM-4 is homologous to TAK-1 (a kinase activated by transforming growth factor beta). LIT-1z is homologous to mitogen-activated protein kinase kinase kinase (MAP3K) and MAP kinase (MAPK)-related NEMO-like kinase (NLK) in mammalian cells. This raised the possibility that TAK-1 and NLK were involved in Wnt signaling in mammalian cells. The authors reported that TAK-1 activation stimulates NLK activity and down-regulates transcriptional activation mediated by beta-catenin and TCF. Injection of NLK suppressed the induction of axis duplication by microinjected beta-catenin in Xenopus embryos. NLK was shown to phosphorylate TCF\LEF factors and inhibit the interaction of the beta-catenin-TCF complex with DNA. Accordingly, the TAK-1-NLK-MAPK-like pathway was shown to negatively regulate the Wnt signaling pathway.

Members of the tumor necrosis factor receptor superfamily have an important role in the induction of cellular signals resulting in cell growth, differentiation, and death. See Smith et al. (1994) Cell 76:959-962. Tumor necrosis factor receptor-1 recruits and assembles a signaling complex containing a number of death domain-containing proteins and a serine/threonine kinase, RIP, that mediates tumor necrosis factor-induced activation of nuclear factor-κB. See Stanger et al. (1995) Cell 81:513-523 and Kelliher et al. (1998) Immunity 8:297-303. Recently, another RIP-like kinase has been characterized, designated “CARDIAK,” which contains a serine/threonine kinase domain as well as a carboxy-terminal caspase recruiting domain (CARD) (Thome, et al. (1998) Current Biology 8:885-888). Overexpression of this serine/threonine kinase induced the activation of both nuclear factor-κB and Jun N-terminal kinase. This kinase also interacted with the tumor necrosis factor receptor-associated factors TRAF-1 and TRAF-2. A dominant negative form of TRAF-2 inhibited CARDIAK-induced nuclear factor-κB activation. The data in the report suggested that CARDIAK is involved in nuclear factor-KB/Jun N-terminal kinase signaling.

Protein kinases play critical roles in cellular growth. Therefore, novel protein kinase polynucleotides and proteins are useful for modulating cellular growth, differentiation and/or development.

Accordingly, protein kinases are a major target for drug action and development. Accordingly, it is valuable to the field of pharmaceutical development to identify and characterize tissues and disorders in which protein kinases are differentially expressed. The present invention advances the state of the art by providing tissues and disorders in which expression of a human protein kinase is relevant. Accordingly, the invention provides methods directed to expression of the protein kinase.

### SUMMARY OF THE INVENTION

It is an object of the invention to identify tissues and disorders in which expression of the protein kinase is relevant.

It is a further object of the invention to provide methods wherein the kinase polypeptides are useful as reagents or targets in protein kinase assays applicable to treatment and diagnosis of disorders mediated by or related to the protein kinase.

It is a further object of the invention to provide methods wherein polynucleotides corresponding to the protein kinase polypeptide are useful as targets or reagents in protein kinase assays applicable to treatment and diagnosis of disorders mediated by or related to the protein kinase.

A specific object of the invention is to identify compounds that act as agonists and antagonists and modulate the expression of the protein kinase in specific tissues and disorders.

A further specific object of the invention is to provide compounds that modulate expression of the protein kinase for treatment and diagnosis of protein kinase-mediated or related disorders.

The invention is thus based on the expression of a human protein kinase in specific tissues and disorders.

The invention provides methods of screening for compounds that modulate expression or activity of the protein kinase polypeptides or nucleic acid (RNA or DNA) in the specific tissues or disorders.

The invention also provides a process for modulating protein kinase polypeptide or nucleic acid expression or activity, especially using the screened compounds.

Modulation may be used to treat conditions related to aberrant activity or expression of the protein kinase polypeptides or nucleic acids.

The invention also provides assays for determining the activity of or the presence or absence of the protein kinase polypeptides or nucleic acid molecules in specific biological samples, including for disease diagnosis.

The invention also provides assays for determining the presence of a mutation in the polypeptides or nucleic acid molecules, including for disease diagnosis.

The invention utilizes isolated protein kinase polypeptides, including a polypeptide having the amino acid sequence shown in SEQ ID NO:2.

The invention also utilizes an isolated protein kinase nucleic acid molecule having the sequence shown in SEQ ID NO: 1.

The invention also utilizes variant polypeptides having an amino acid sequence that is substantially homologous to the amino acid sequence shown in SEQ ID NO:2.

The invention also utilizes variant nucleic acid sequences that are substantially homologous to the nucleotide sequence shown in SEQ ID NO: 1.

The invention also utilizes fragments of the polypeptide shown in SEQ ID NO:2 and nucleotide sequence shown in SEQ ID NO: 1, as well as substantially homologous fragments of the polypeptide or nucleic acid.

The invention further utilizes nucleic acid constructs comprising the nucleic acid molecules described herein. In a preferred embodiment, the nucleic acid molecules of the invention are operatively linked to a regulatory sequence.

The invention also utilizes vectors and host cells that express the protein kinase and provides methods for expressing the protein kinase nucleic acid molecules and polypeptides in specific cell types and disorders, and particularly recombinant vectors and host cells.

The invention also utilizes methods of making the vectors and host cells and provides methods for using them to assay expression and cellular effects of expression of the protein kinase nucleic acid molecules and polypeptides in specific cell types and disorders.

The invention also utilizes antibodies or antigen-binding fragments thereof that selectively bind the protein kinase polypeptides and fragments.

### DESCRIPTION OF THE DRAWINGS

FIG. 1 shows the 20893 nucleotide sequence (SEQ ID NO:1) and the deduced amino acid sequence (SEQ ID NO:2). The coding sequence for 20893 is set forth in SEQ ID NO:3. BLAST analysis showed significant scores to rat 5′ AMP-activated protein kinase (NCBI Accession N. U40819).

FIG. 2 shows expression of the gene in various normal human tissues. Expression levels were determined by quantitative RT-PCR (Taqman® brand quantitative PCR kit, Applied Biosystems). The quantitative RT-PCR reactions were performed according to the kit manufacturer's instructions.

FIG. 3 shows expression in various human tissues and in HepG2 (hepatocyte) cell lines and HepG2 cell lines infected with the hepatitis B virus (2.15). CD3 cells were treated with phytohaemagglutinin (PHA). Expression levels were determined as described in the description of FIG. 2.

FIG. 4 shows increased expression of the gene in HepG2 cell lines and HepG2 cell lines infected with the hepatitis B virus. Expression levels were determined as described in the description of FIG. 2.

FIG. 5 shows expression of the 20893 protein kinase in various human tissues and in a liver fibrosis model. Hepatic stellate cells are a paradigm for liver fibrosis. Stellate/FBS (fetal bovine serum). NHLF: normal human lung fibroblasts. NHLF TGF: normal human lung fibroblasts treated with transforming growth factor β. Liver fibrosis: fibrotic liver biopsies. Expression levels were determined as described in the description of FIG. 2.

FIG. 6 shows expression of the 20893 protein kinase in various human tissues, hepatic stellate cells, activated hepatic stellate cells, normal human dermal fibroblasts with and without transforming growth factor β treatment, normal human lung fibroblasts with and without transforming growth factor β treatment, and fibrotic liver biopsies (LF/NDR) compared to non-fibrotic liver samples. Expression levels were determined as described in the description of FIG. 2.

FIG. 7 depicts relative expression of 20893 in various human tissues: lung (column 1); kidney (column 2); brain (column 3); heart (column 4); colon (column 5); tonsil (column 6); liver pool, pool of 7 normal livers (column 7); fetal liver (column 8); spleen (column 9); ThI cells (column 10); Th2 cells, 48 hrs (column 11); Th1 cells (column 12); Th2 cells, 48 hrs (column 13); HepG2-A, immortalized hepatocyte cells (column 14); HepG2.2.15-A, HepG2 stably transfected with hepatitis B virus (column 15); monocytes (column 16); monocytes stimulated with lipopolysaccharide (column 17); resting stellate cells (column 18), serum-reactivated stellate cells (column 19); normal human lung fibroblasts, NHLF (column 20); NHLF treated with TGF-β (column 21); liver fibrosis (columns 22-25); Th0, 6 hr (column 26); Th2, 6 hr (column 27), CD19, B cells (column 28); granulocytes (column 29); PBMC, resting peripheral blood mononuclear cells (column 30); PBMC cells treated with PHA (column 31); PBMC cells treated with interleukon-10 (IL-10) and interleukon-4 (IL-4) (column 32); PBMC cells treated with tumor necrosis factor-α (TNF-α) and ILFN-g (column 33); NHBE, normal human bronchial epithelial (column 34); NHBE treated with IL13-2 (column 35); bone marrow mononuclear cells (column 36); CD34+cells from mPB, mobilized peripheral blood (column 37); CD34+cells from ABM, adult bone marrow (column 38); erythroid cells (column 39); megakaryocytes, day 7 (column 40); megakaryocytes, day 10 (column 41); megakaryocytes, day 14 (column 42); neutrophil, day 7 (column 43); CD11b-cells from human mobilized bone marrow (column 44); glycophorin A positive cells from human bone marrow (column 45); erythroid, day 6 (column 46). Expression levels were determined as described in the description of FIG. 2.

FIG. 8 depicts 20893 expression in clinical samples of normal human liver tissue: liver pool, liver NDR 200/2, liver CHT and liver PIT, and the following human fibrotic liver tissues: LF/NDR 079, LF/NDR 141, LF/NDR 156, LF/NDR 190, LF/NDR 191, LF/NDR 192, LF/NDR 194, LF/NDR 225, and LF/MPI 447/448. Expression levels were determined as described in the description of FIG. 2.

FIG. 9 depicts 20893 expression in a variety of human tissue samples: lung (column 1); kidney (column 2); colon (column 3); heart (column 4); spleen (column 5); CD3 cells (column 6); CD3 cells treated with PHA (column 7); granulocytes (column 8); stellate cells harvested after first passage (column 9); resting stellate cells (column 10); serum reactivated stellate cells (column 11); NHDF, normal human dermal fibroblasts (column 12); NHDF treated with TGF-β (column 13); NHLF (column 14); NHLF treated with TGF-β (column 15); HepG2 (column 16); HepG2 treated with TGF-β (column 17); cultured human hepatocytes (column 18); hepatocytes treated with PMA and ionomycin (column 19); hepatocytes treated with TGF-β for 24 hrs (column 20); hepatocytes treated with TGF-β for 48 hrs (column 21); liver pool (column 21); LF NDR 200-2, normal liver (column 22); LF CHT 339-4, normal liver (column 23); liver pit 260, normal liver (column 24); fibrotic liver samples: LF/NDR 079 (column 25); LF/NDR 141 (column 26); LF/NDR 156 (column 27); LF/NDR 190 (column 28); LF/NDR 191 (column 29); LF/NDR 192 (column 30); LF/NDR 194 (column 31); LF/NDR 225 (column 32); and LF/MPI 447/448 (column 33). Expression levels were determined as described in the description of FIG. 2.

FIG. 10 depicts 20893 expression in a variety of human tissues, particularly liver fibrosis. The tissues include: kidney (column 1); heart (column 2); liver pool (column 3); LF NDR 200-2 (column 4); liver pit 260 (column 5); LFINDR 079 (column 6); LF/NDR 190 (column 7), LF/NDR 191 (column 8); freshly harvested stellate cells (column 9); stellate cells harvested after first passage (column 10); resting stellate cells (column 11); serum reactivated stellate cells (column 12); non cultured hepatocytes (column 13); cultured human hepatocytes (column 14); hepatocytes treated with PMA and ionomycin (column 15); hepatocytes treated with TGF-β for 24 hrs (column 16); hepatocytes treated with TGF-β for 48 hrs (column 17); and an NTC reference (column 18). Expression levels were determined as described in the description of FIG. 2.

FIG. 11 depicts 20893 expression in rat 5.5 weeks serum model experiments. The first 4 columns contain control serum data while the remaining columns contain fibrotic serum data. Expression levels were determined as described in the description of FIG. 2.

FIG. 12 depicts 20893 expression in rats three weeks after bile duct ligation (BDL) induced fibrosis. Columns 2-11 contain data from fibrotic liver while the remaining columns contain data from control specimens. Expression levels were determined as described in the description of FIG. 2.

FIG. 13 depicts 20893 expression in rats after CCl₄ induction of fibrosis. Expression levels were determined as described in the description of FIG. 2.

FIG. 14 depicts 20893 expression over time in hepatic stellate cells and control tissues. The control tissues include: heart, kidney, brain, and lung. Expression data from an initial time point, day 1, day 3 and day 7 are indicated also. Expression levels were determined as described in the description of FIG. 2.

FIG. 15 depicts the alignment of the protein kinase domain of human 20893 with a consensus amino acid sequence derived from a hidden Markov model. The lower sequence is the consensus amino acid sequence (SEQ ID NO:4), while the upper amino acid sequence corresponds to amino acids 55 to 350 of SEQ ID NO:2.

### DETAILED DESCRIPTION OF THE INVENTION

The present invention is based, at least in part, on methods for using a protein or nucleic acid molecule, referred to herein as a “kinase” or “protein kinase” nucleic acid and polypeptide molecule, and which is a serine-threonine protein kinase. In embodiments pertaining to the polypeptide or nucleic acid of the invention, this polypeptide is a protein kinase and the nucleic acid encodes it. The kinase of the invention plays a role in, or functions in, signaling pathways associated with cellular growth and/or cellular metabolic pathways, and, in the present case, is involved in a productive viral infection. These growth and metabolic pathways are described in Lodish et al. (1995) Molecular Cell Biology (Scientific American Books Inc., New York, N.Y.) and Stryer Biochemistry, (W. H. Freeman, New York), the contents of which are incorporated herein by reference. In one embodiment, the protein kinase molecule modulates the activity of one or more cellular components involved in viral infection, cellular growth, or differentiation, e.g., HBV-infected cells. In another embodiment, the protein kinase molecule of the present invention is capable of modulating the phosphorylation state of a kinase molecule or the phosphorylation state of one or more proteins involved in viral infection, cellular growth, or differentiation, e.g., HBV-infected cells. See also Lodish et al. and Stryer, supra. In another embodiment the protein kinase modulates the activity of one or more cellular components involved in tissue fibrosis, especially liver fibrosis, and more particularly fibrosis associated with viral infections. In addition, protein kinases of the present invention are targets of drugs described in Goodman and Gilman (1996), The Pharmacological Basis of Therapeutics (9^{th} ed.) Hartman & Limbard Editors, the contents of which are incorporated herein by reference. Particularly, the protein kinase of the invention modulates phosphorylation in HBV virus-infected tissues and cells, such as liver.

As used herein, the term “kinase” includes a protein or polypeptide that is capable of modulating its own phosphorylation state or the phosphorylation state of a different protein or polypeptide. Kinases can have a specificity for (i.e., a specificity to phosphorylate) serine/threonine residues, tyrosine residues, or both serine/threonine and tyrosine residues, e.g., the dual-specificity kinases. As referred to herein, kinases, such as protein kinases, preferably include a catalytic domain of about 200-400 amino acid residues in length, preferably about 200-300 amino acid residues in length, or more preferably about 250-300 amino acid residues in length, which includes preferably 5-20, more preferably 5-15, or most preferably 11 highly conserved motifs or subdomains separated by sequences of amino acids with reduced or minimal conservation. Specificity of a kinase for phosphorylation of either tyrosine or serine/threonine can be predicted by the sequence of two of the subdomains (Vib and VIII) in which different residues are conserved in each class (as described in, for example, Hanks et al. (1988) Science 241:42-52, the contents of which are incorporated herein by reference). These subdomains are also described in further detail herein.

Plasmids containing the nucleotide sequences of the invention were deposited with the Patent Depository of the American Type Culture Collection (ATCC), Manassas, Va., on Jul. 7, 2000, and assigned Patent Deposit No. PTA-2201. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. §112. The nucleotide sequences utilized in the methods of the invention are indicated in SEQ ID NO: 1 and SEQ ID NO: 3 comprising nucleotides 1381-3366 of SEQ ID NO: 1; SEQ ID NO: 1 and SEQ ID NO: 3 encode the amino acid sequence listed in SEQ ID NO: 2.

Protein kinases play a role in signaling pathways associated with cellular growth. For example, protein kinases are involved in the regulation of signal transmission from cellular receptors, e.g., growth-factor receptors, entry of cells into mitosis, and the regulation of cytoskeleton function, e.g., actin bundling.

Assays for measuring protein kinase activity are well known in the art depending on the particular protein kinase. Specific assay protocols are available in standard sources known to the ordinarily skilled artisan. For example, see “Kinases” in Ausubel et al., eds. (1994-1998) Current Protocols in Molecular Biology (3) and references cited therein; http://www.sdsc.edu/Kinases/pkr/pk_protocols.html; and http://www.sdsc.edu/Kinases/pkr/pk_protocols/tyr_synpep_assay.html.

Inhibition or over-stimulation of the activity of protein kinases involved in signaling pathways associated with cellular growth can lead to perturbed cellular growth, which can in turn lead to cellular growth related-disorders. As used herein, a “cellular growth-related disorder” includes a disorder, disease, or condition characterized by a deregulation, e.g., an upregulation or a downregulation, of cellular growth. Cellular growth deregulation may be due to a deregulation of cellular proliferation, cell cycle progression, cellular differentiation and/or cellular hypertrophy. Examples of cellular growth related disorders include cardiovascular disorders such as heart failure, hypertension, atrial fibrillation, dilated cardiomyopathy, idiopathic cardiomyopathy, or angina; proliferative disorders or differentiative disorders such as cancer, e.g., liver cancer, melanoma, prostate cancer, cervical cancer, breast cancer, colon cancer, or sarcoma. Disorders associated with virally-infected cells or tissues are also encompassed. The compositions are useful for the treatment of viral infection, such as DNA virus infection, including but not limited to HBV. They are also useful for treating or preventing tissue fibrosis, especially liver fibrosis, and more particularly fibrosis associated with virus infection.

As used herein, a “signaling pathway” refers to the modulation (e.g., stimulation or inhibition) of a cellular function/activity upon the binding of a ligand to a receptor. Examples of such functions include mobilization of intracellular molecules that participate in a signal transduction pathway, e.g., phosphatidylinositol 4,5-bisphosphate (PIP2), inositol 1,4,5-triphosphate (IP3) and adenylate cyclase; polarization of the plasma membrane; production or secretion of molecules; alteration in the structure of a cellular component; cell proliferation, e.g., synthesis of DNA; cell migration; cell differentiation; and cell survival.

The response depends on the type of cell. In some cells, binding of a ligand to the receptor may stimulate an activity such as release of compounds, gating of a channel, cellular adhesion, migration, differentiation, etc., through phosphatidylinositol or cyclic AMP metabolism and turnover while in other cells, binding will produce a different result.

The cAMP turnover pathway is a signaling pathway. As used herein, “cyclic AMP turnover and metabolism” refers to the molecules involved in the turnover and metabolism of cAMP as well as to the activities of these molecules. Cyclic AMP is a second messenger produced in response to ligand-induced stimulation of certain receptors. In the cAMP signaling pathway, binding of a ligand can lead to the activation of the enzyme adenyl cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

The cGMP turnover pathway is also a signaling pathway. As used herein, “cyclic GMP turnover and metabolism” refers to the molecules involved in the turnover and metabolism of cGMP as well as to the activities of these molecules. Cyclic GMP is a second messenger produced in response to ligand-induced stimulation of certain receptors. In the cGMP signaling pathway, binding of a ligand can lead to the activation of the enzyme guanyl cyclase, which catalyzes the synthesis of cGMP. Synthesized cGMP can in turn activate a cGMP-dependent protein kinase.

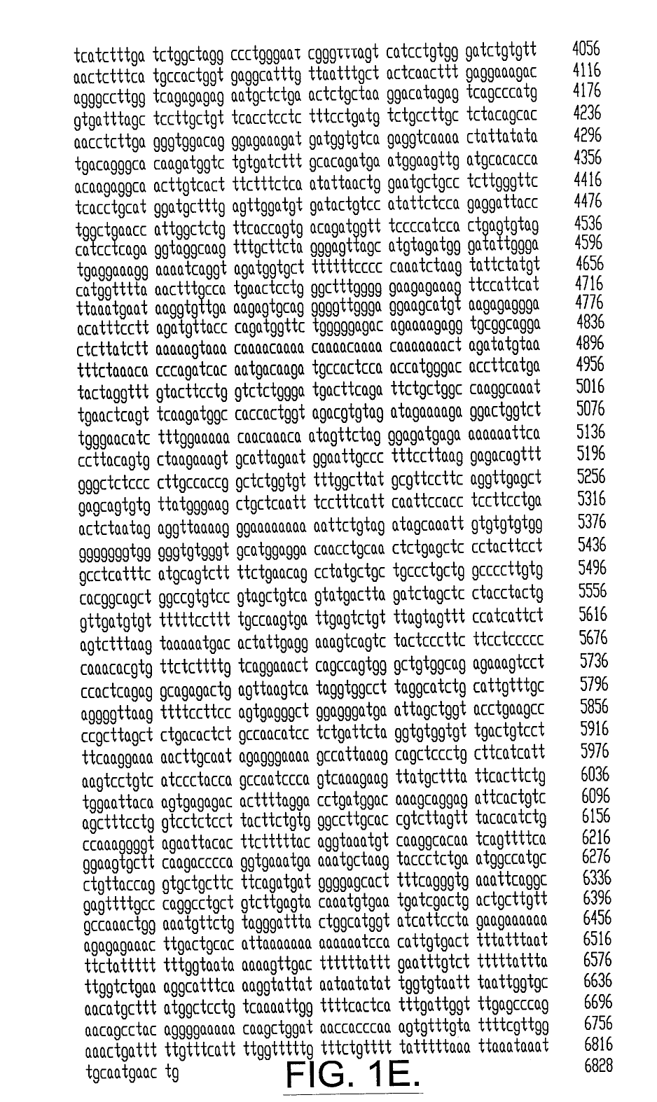

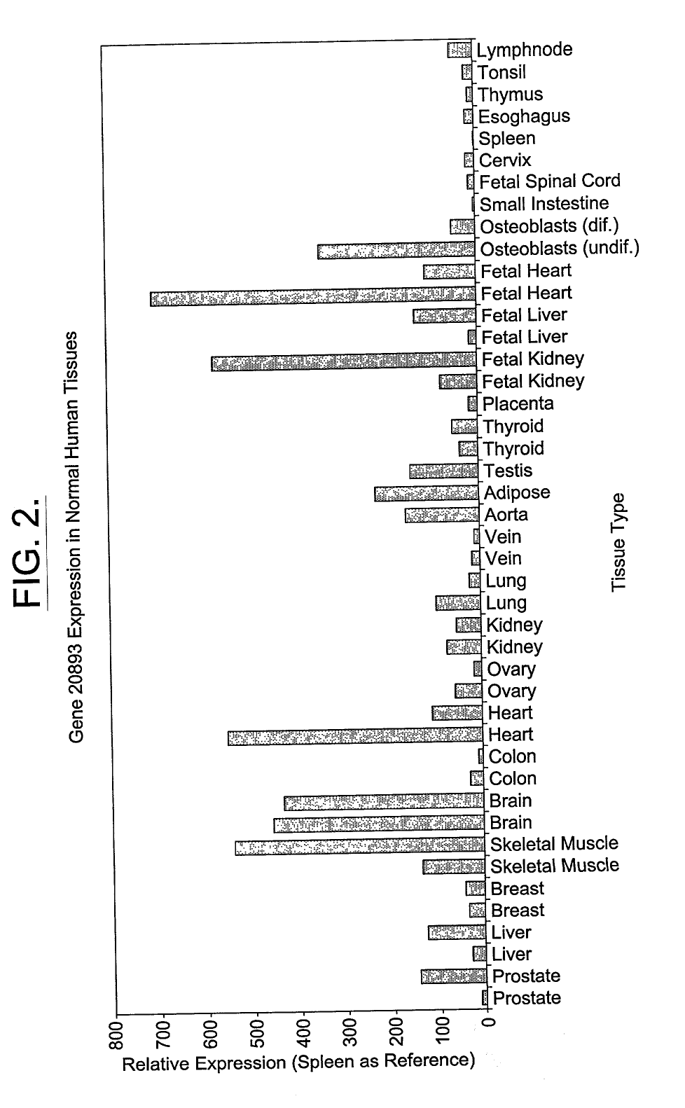

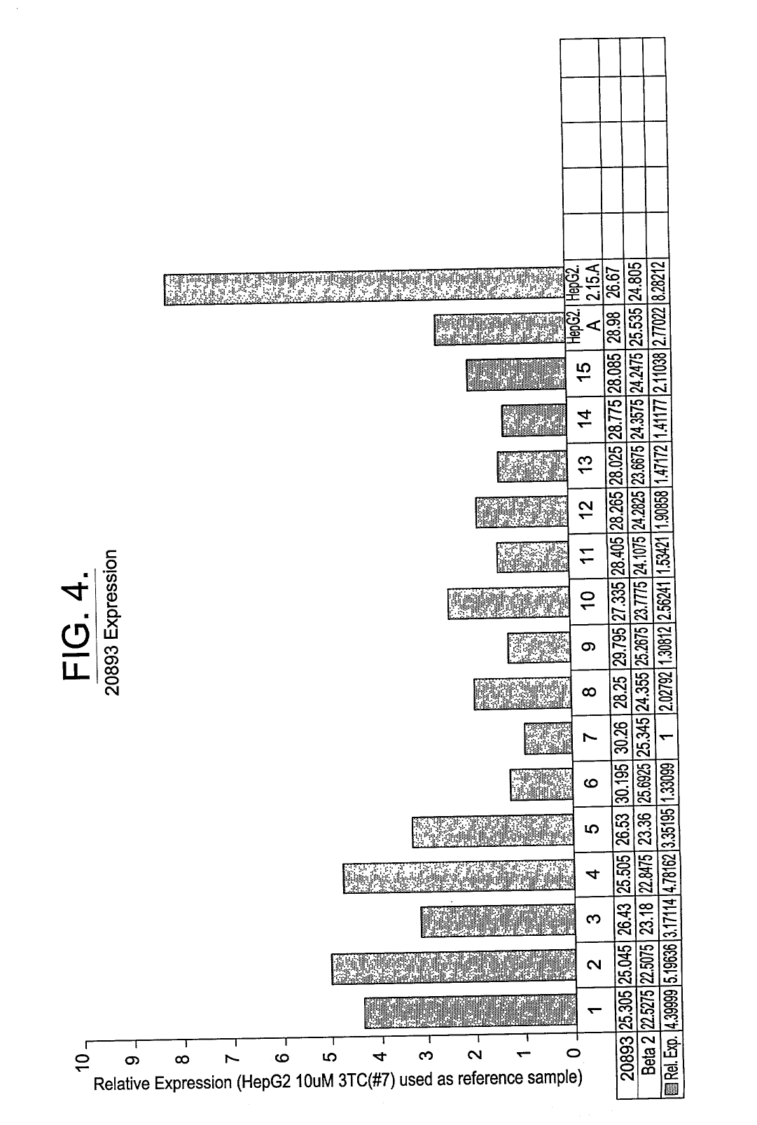

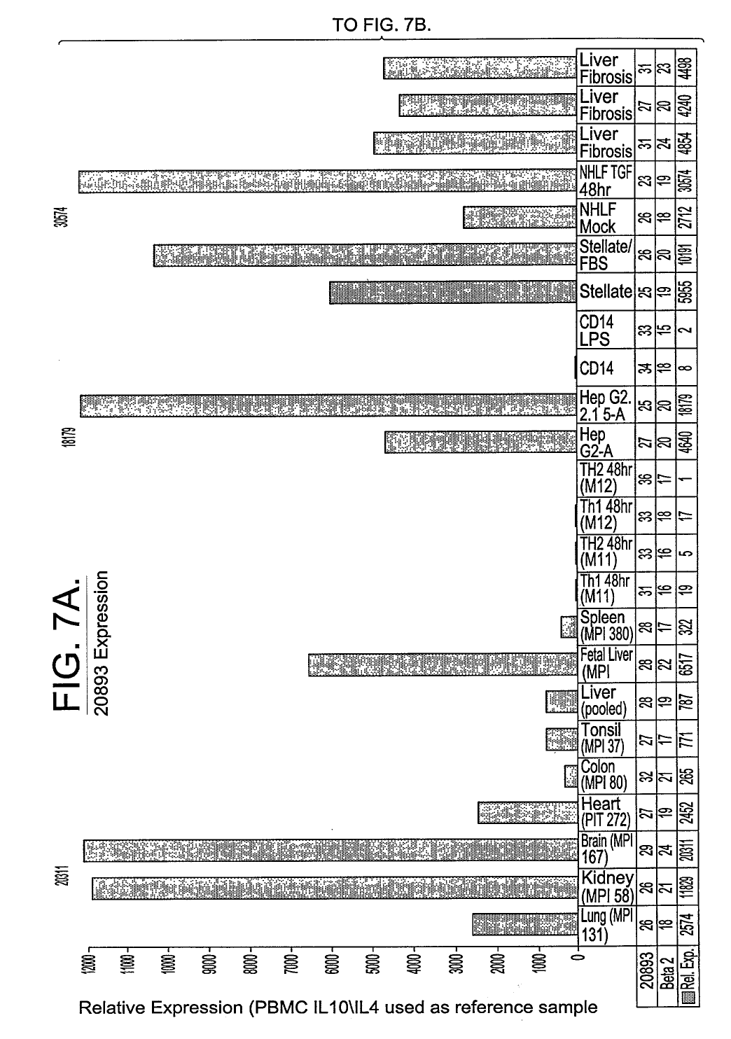

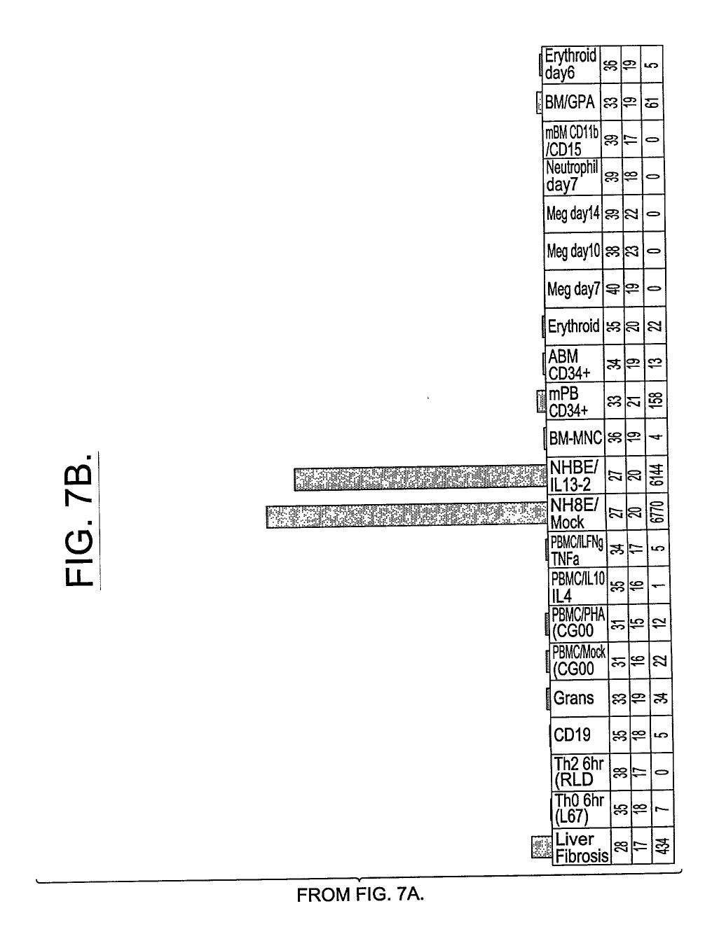

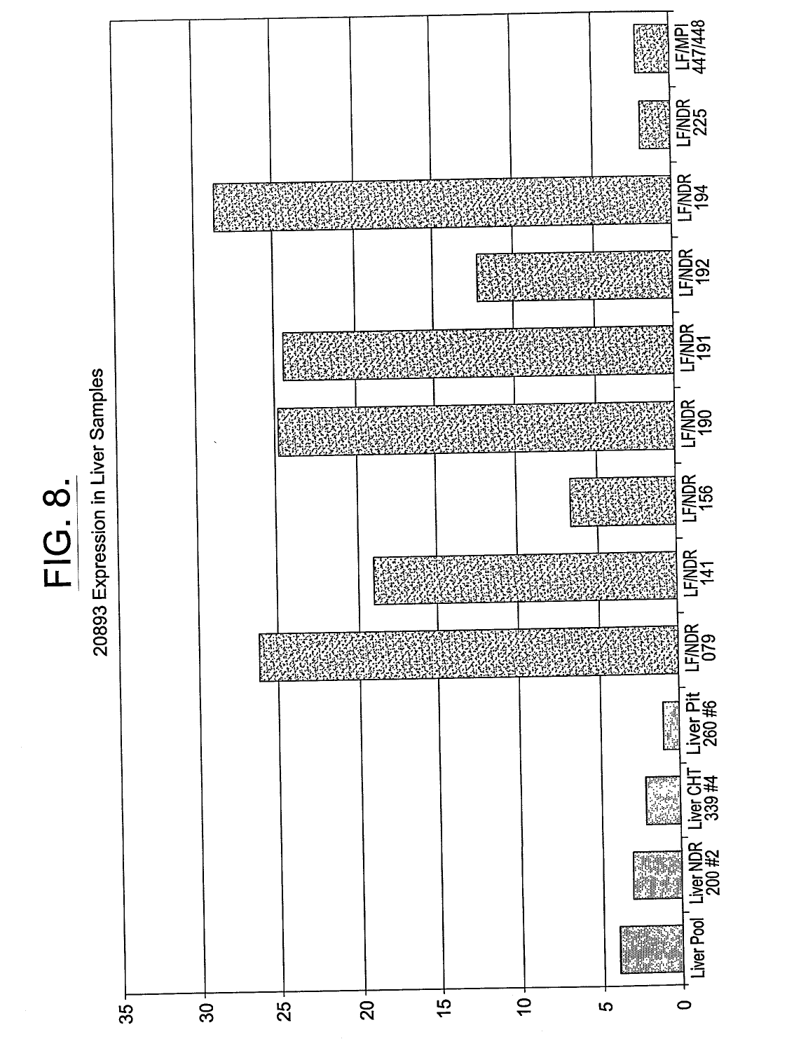

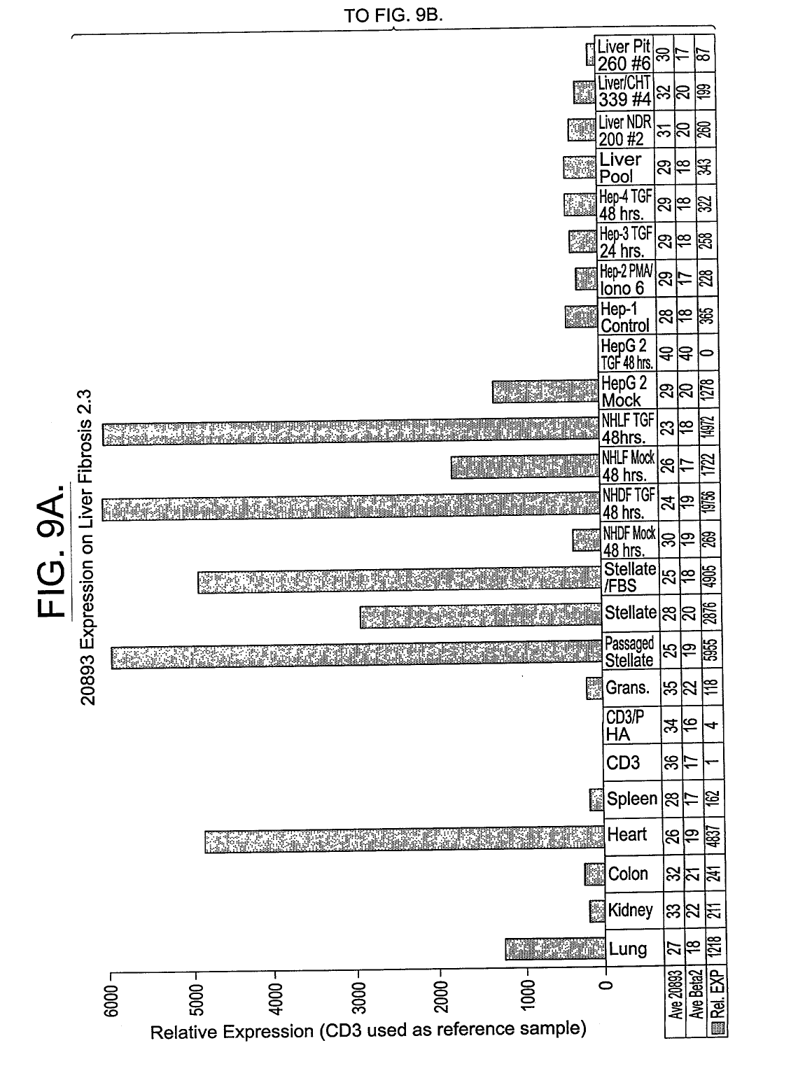

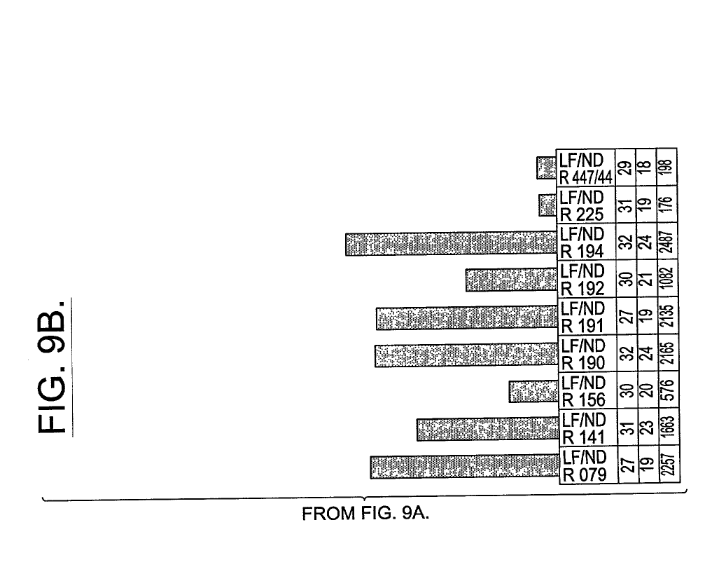

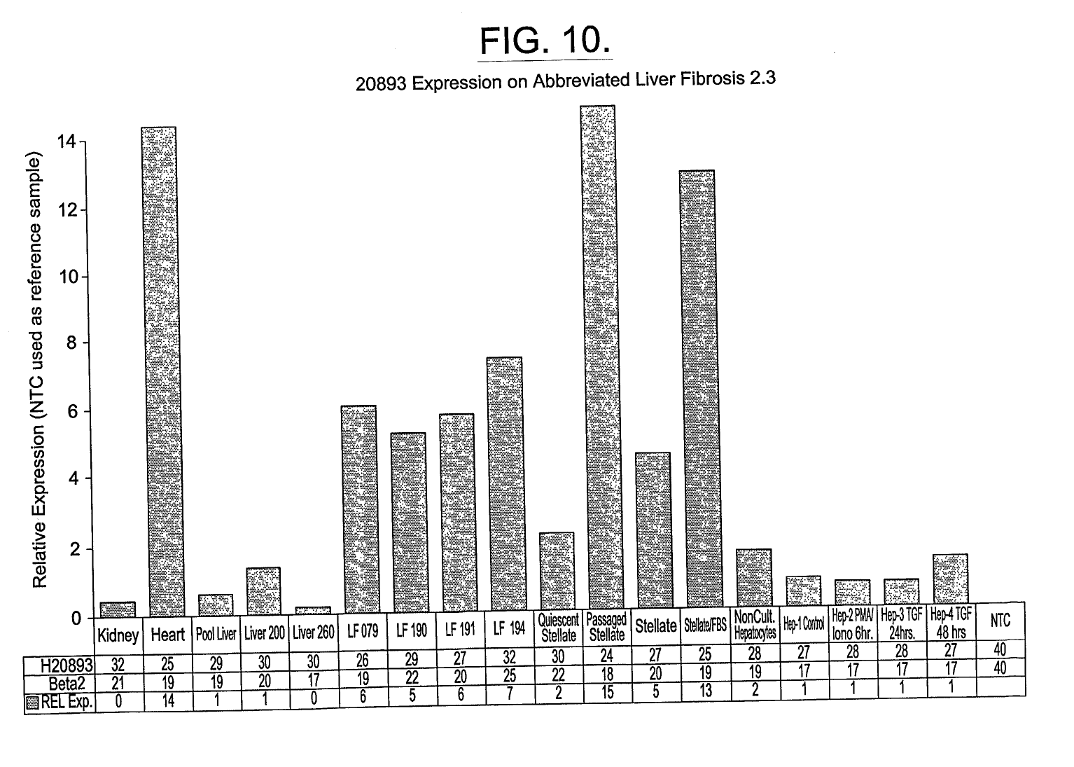

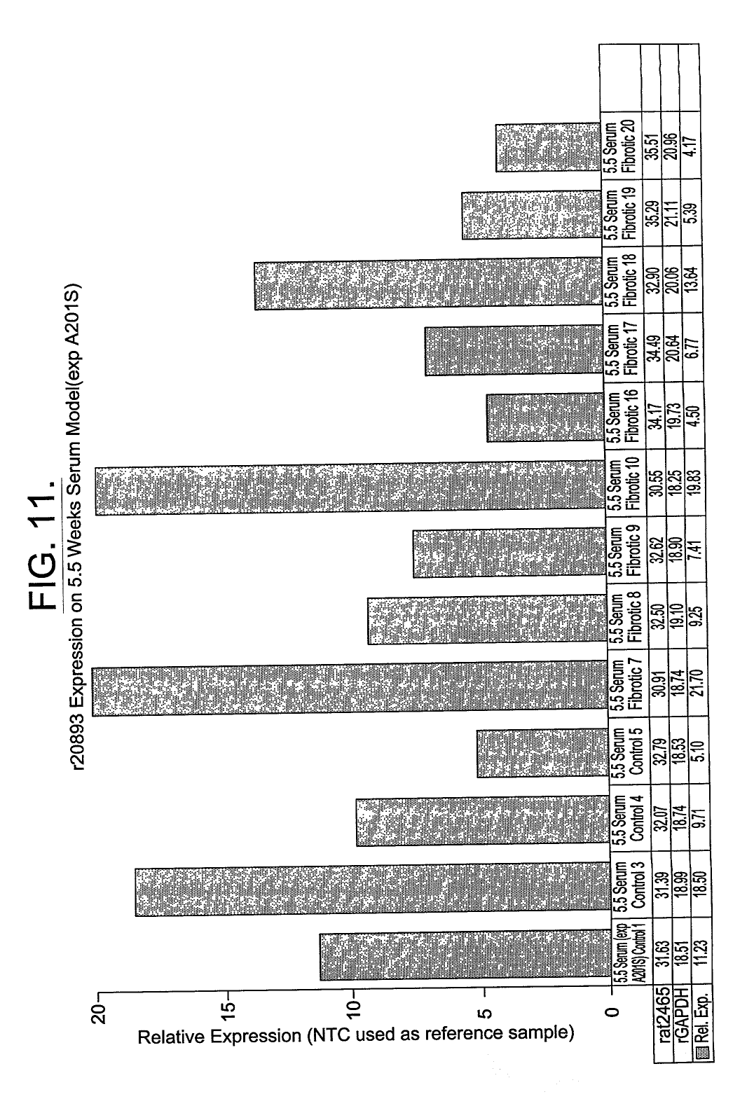

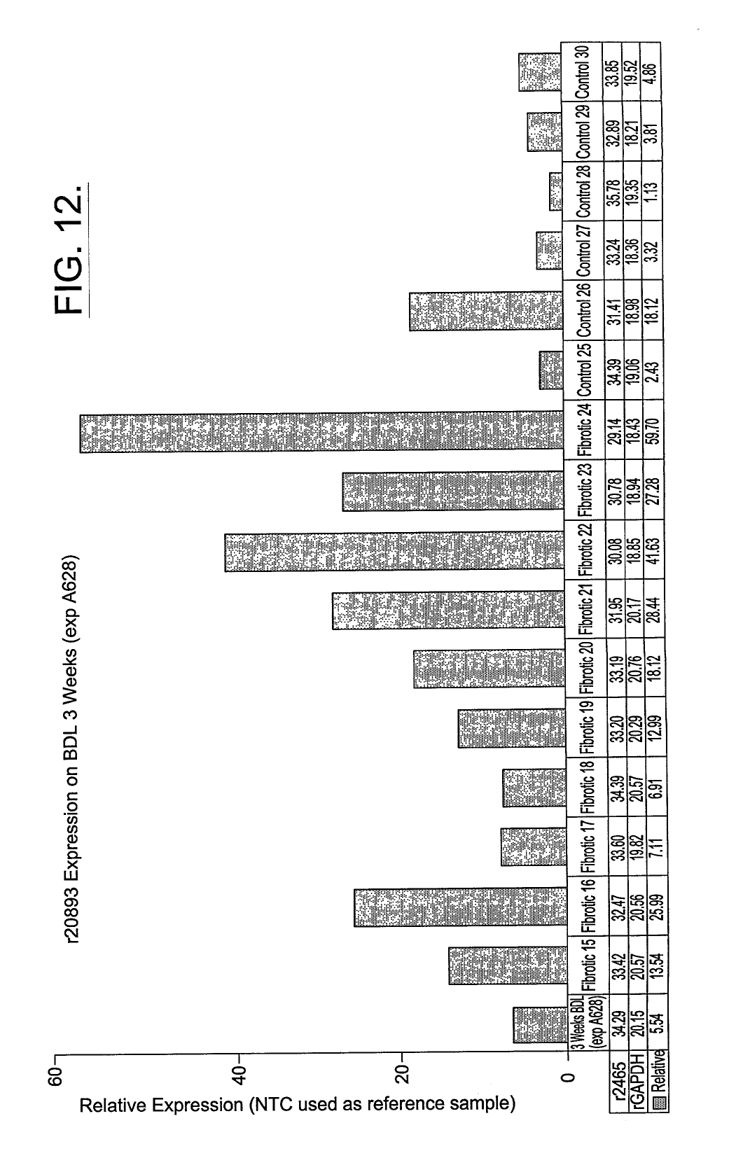

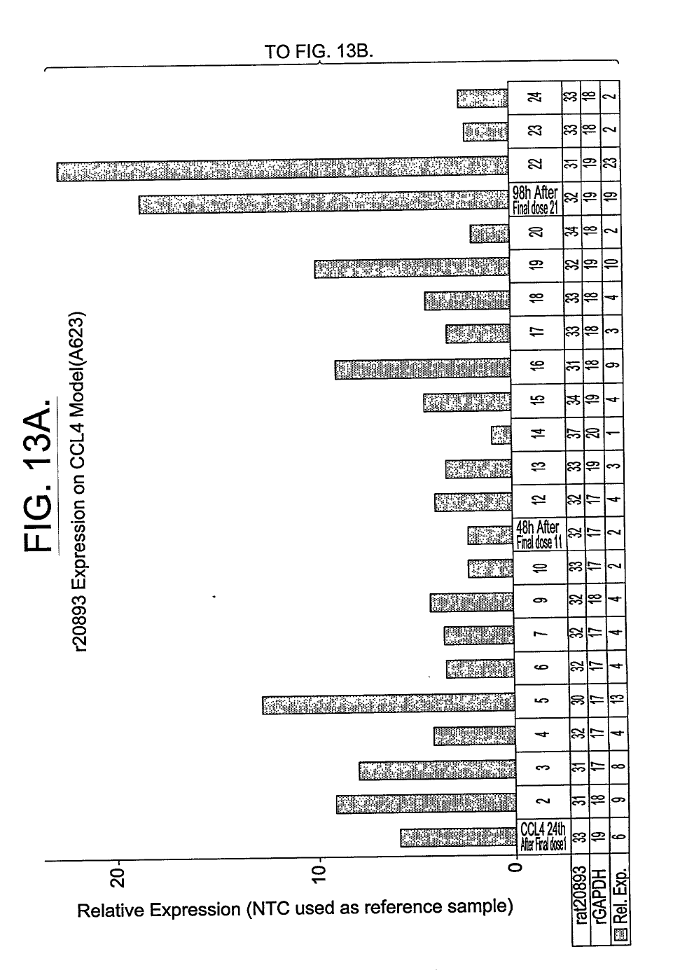

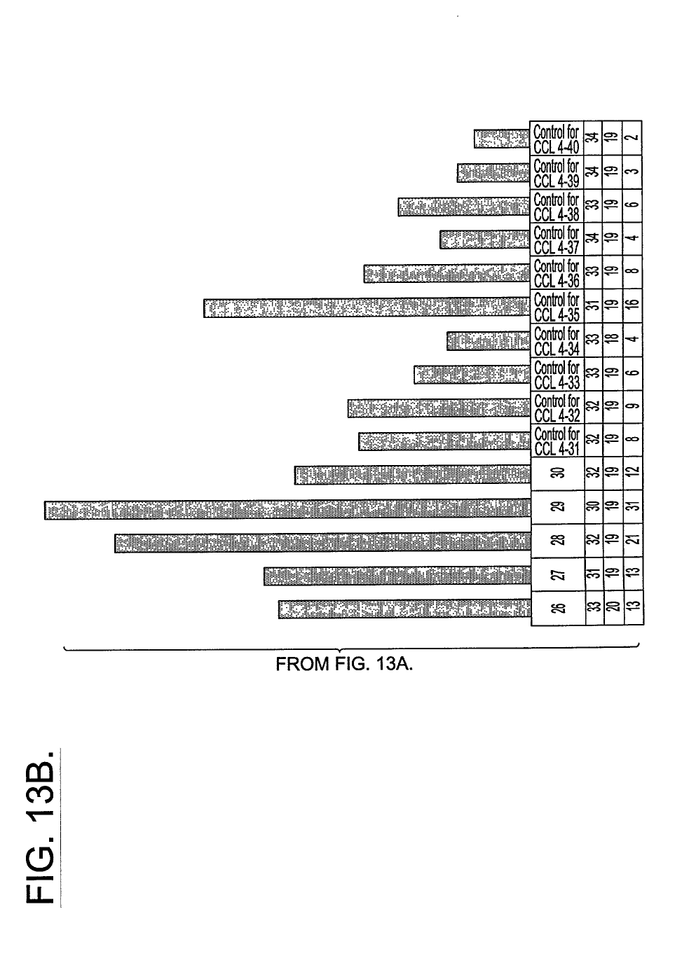

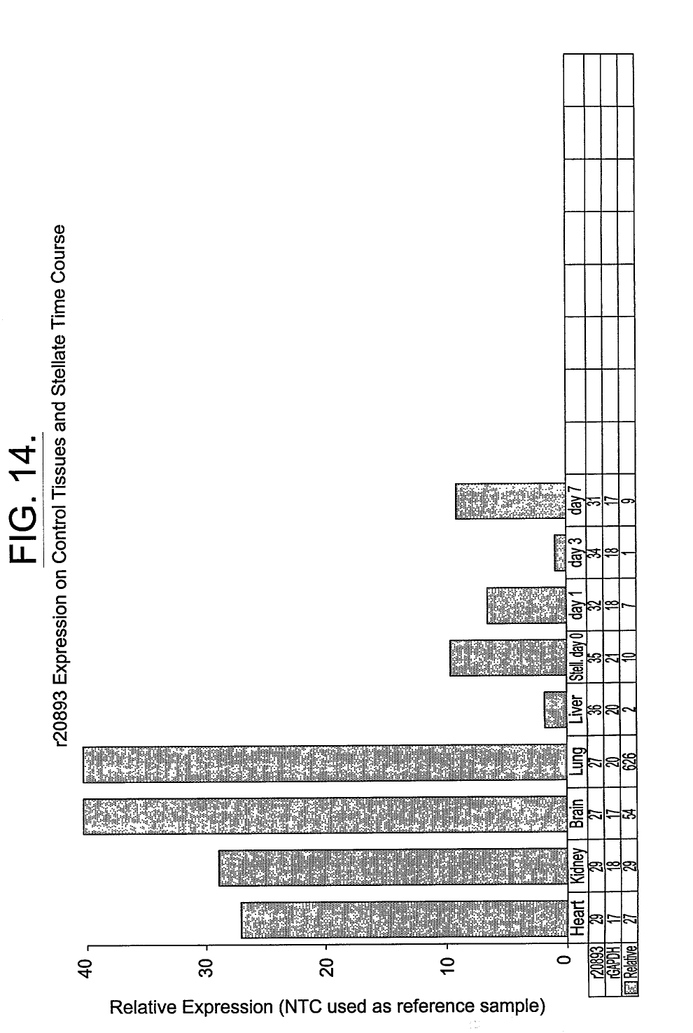

The invention is directed to methods, uses and reagents applicable to methods and uses that are applied to cells, tissues and disorders of these cells and tissues wherein the 20893 protein kinase expression is relevant. The protein kinase is expressed in a variety of tissues as shown in FIGS. 2-14. Accordingly, the methods and uses of the invention as disclosed in greater detail below apply to these tissues, disorders involving these tissues, and particularly to the disorders with which gene expression is associated, as shown in these figures and as disclosed herein. Accordingly, the methods, uses and reagents disclosed in greater detail below especially apply to skeletal muscle, brain, heart, fetal kidney, fetal heart and osteoblasts. They also especially apply to virus-infected cells and particularly to virus-infected liver cells, more particularly to liver cells infected by hepatitis B virus. They also especially apply to conditions in which tissue fibrosis, such as lung and liver fibrosis, have developed or may develop. Accordingly, the uses, reagents and methods disclosed in detail herein below apply especially to these tissues, cell types, and disorders.

In addition, 20893 is expressed in a variety of endothelial and vascular cell types. HUVEC formation of tubes on Matrigel media serves as a model of angiogenesis. 20893 is upregulated during tube formation Matrigel, indicating a role in angiogenesis. The angiogenic role of 20893 was confirmed by examining expression of 20893 in mouse ischemic models of angiogenesis. Ischemic hindlimbs of mice exhibit higher levels of 20893 expression than control hindlimbs. 20893 expression was also examined in an apoE mouse atherosclerosis model. 20893 expression increased in the apoE atherosclerotic mouse compared with the wild-type mouse indicating a role in atherosclerosis.

Methods of Using the Polypeptide

The invention provides methods using the protein kinase, variants, or fragments, including but not limited to use in the cells, tissues, and disorders as disclosed herein.

The invention provides biological assays related to protein kinases. Such assays involve any of the known functions or activities or properties useful for diagnosis and treatment of protein kinase-related conditions. These include, but are not limited to, interaction with and phosphorylation of substrate (polypeptide or other macromolecule), ability to be bound by specific antibody, GTP or ATP, GMP or AMP binding, effector molecule interaction as well as the various other properties and functions disclosed herein and disclosed in the references cited herein.

The invention provides drug screening assays, in cell-based or cell-free systems. Cell-based systems can be native, i.e., cells that normally express the kinase, as a biopsy, or expanded in cell culture. In one embodiment, cell-based assays involve recombinant host cells expressing the kinase. Accordingly, cells that are useful in this regard include, but are not limited to, those disclosed herein as expressing or differentially expressing the kinase, such as those shown in FIGS. 2-14. Such cells can naturally express the gene or can be recombinant, containing one or more copies of exogenously-introduced kinase sequences or genetically modified to modulate expression of the endogenous kinase sequence.

This aspect of the invention particularly relates to cells derived from subjects with disorders involving the tissues in which the kinase is expressed or derived from tissues subject to disorders including, but not limited to, those disclosed herein. These disorders may naturally occur, as in populations of human subjects, or may occur in model systems such as in vitro systems or in vivo, such as in non-human transgenic organisms, particularly in non-human transgenic animals.

Such assays can involve the identification of agents that interact with the kinase protein. This interaction can be detected by functional assays, such as the ability to be activated or otherwise affected by an effector molecule, such as phosphorylation by an effector or phosphorylating a substrate. Such interaction can also be measured by ultimate biological effects, such as increasing or decreasing the levels of ATP or GTP, increasing or decreasing the levels of phosphorylated substrate, or having biological effects on immunity/inflammation, angiogenesis, atherosclerosis, cell proliferation, viral infection, or tissue fibrosis, particularly lung or liver fibrosis, especially virus-related fibrosis, and in vitro markers for fibrosis.

Determining the ability of the test compound to interact with the kinase can also comprise determining the ability of the test compound to preferentially bind to the polypeptide as compared to the ability of a known binding molecule (e.g., ATP, GTP, GMP or AMP) to bind to the polypeptide.

In yet another aspect of the invention, the invention provides methods to identify proteins that interact with the kinase in the tissues and disorders disclosed. The proteins of the invention can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins (captured proteins) which bind to or interact with the proteins of the invention and modulate their activity.

The invention provides methods to identify compounds that modulate kinase activity. Such compounds, for example, can increase or decrease affinity or rate of binding to ATP or AMP, compete with ATP or AMP for binding to the kinase, or displace ATP or AMP bound to the kinase. Such compounds can also, for example, increase or decrease affinity or rate of binding to substrate or effector molecule, compete with substrate or effector molecule for binding, or displace substrate or effector molecule bound to the kinase. Both kinase and appropriate variants and fragments can be used in high-throughput screens to assay candidate compounds for the ability to bind to the kinase. These compounds can be further screened against a functional kinase to determine the effect of the compound on the kinase activity. Compounds can be identified that activate (agonist) or inactivate (antagonist) the kinase to a desired degree. Modulatory methods can be performed in vitro (e.g., by culturing the cell with the agent) or, alternatively, in vivo (e.g., by administering the agent to a subject. The subject can be a human subject, for example, a subject in a clinical trial or undergoing treatment or diagnosis, or a non-human transgenic subject, such as a transgenic animal model for disease.

Treatment is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. “Subject”, as used herein, can refer to a mammal, e.g. a human, or to an experimental or animal or disease model. The subject can also be a non-human animal, e.g. a horse, cow, goat, or other domestic animal. A therapeutic agent includes, but is not limited to, small molecules, peptides, antibodies, ribozymes and antisense oligonucleotides.

The invention provides methods to screen a compound for the ability to stimulate or inhibit interaction between the kinase protein and a target molecule that normally interacts with the kinase protein. The target can be an effector molecule, such as that which phosphorylates the kinase, a component of a signal pathway with which the kinase protein normally interacts (for example, substrate protein, ATP, AMP, or other interactor involved in the kinase pathway). The assay includes the steps of combining the kinase protein with a candidate compound under conditions that allow the kinase protein or fragment to interact with the target molecule, and to detect the formation of a complex between the kinase protein and the target, or to detect the biochemical consequence of the interaction with the kinase and the target, such as any of the associated effects of signal transduction, including but not limited to, substrate protein phosphorylation, including, but not limited to, phosphorylation of a transcription factor, modulation of transcription as a result of regulated transcription factor, autophosphorylation, interaction with an effector molecule of the pathway, ATP or GTP turnover, and biological endpoints of the pathway.

Determining the ability of the kinase to bind to a target molecule can also be accomplished using a technology such as real-time Bimolecular Interaction Analysis (BIA). Sjolander et al. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BLAcore™). Changes in the optical phenomenon surface plasmon resonance (SPR) can be used as an indication of real-time reactions between biological molecules.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145).

Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. 97:6378-6382); (Felici (1991) J. Mol. Biol 222:301-310); (Ladner supra).

Candidate compounds include, for example, 1) peptides such as soluble peptides, including Ig-tailed fusion peptides and members of random peptide libraries (see, e.g., Lam et al. (1991) Nature 354:82-84; Houghten et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

One candidate compound is a soluble full-length kinase or fragment that competes for substrate macromolecule, e.g., peptide, or effector (for example upstream kinase) binding. Other candidate compounds include mutant kinases or appropriate fragments containing mutations that affect kinase function and thus compete for substrate or effector molecule. Accordingly, a fragment that competes for effector or substrate, for example with a higher affinity, or a fragment that binds substrate or effector but does not phosphorylate or is not phosphorylated by these molecules (respectively), is encompassed by the invention.

Another candidate compound is a soluble full-length kinase or fragment that competes for ATP, GTP, GMP or AMP binding. Other candidate compounds include mutant kinases or appropriate fragments containing mutations that affect kinase function and thus compete for ATP, GTP, GMP or AMP. Accordingly, a fragment that competes for ATP, GTP, AMP or GMP, for example with a higher affinity, or a fragment that binds AMP or GMP but is not activated by it, is encompassed by the invention.

The invention provides other end points to identify compounds that modulate (stimulate or inhibit) kinase activity. The assays typically involve an assay of events in a signal transduction pathway or other pathway in which the kinase is involved that indicate kinase activity. Thus, the expression of genes that are up- or down-regulated in response to the kinase dependent signal cascade or other cascade can be assayed. In one embodiment, the regulatory region of such genes can be operably linked to a marker that is easily detectable, such as luciferase. Alternatively, phosphorylation of the kinase, or a kinase target, could also be measured.

Any of the biological or biochemical functions mediated by the kinase can be used as an endpoint assay. These include all of the biochemical or biocherical/biological events described herein, in the references cited herein, incorporated by reference for these endpoint assay targets, and other functions known to those of ordinary skill in the art.

In the case of the kinase, specific end points can include AMP and GMP activation of the kinase, phosphorylation of a substrate for the kinase, autophosphorylation of the kinase, or phosphorylation of the kinase by an upstream effector (e.g., kinase) molecule.

Assays for kinase function include, but are not limited to, those that are well known in the art and available to the person of ordinary skill in the art, for example, those in the references referred to above, and which are incorporated herein by reference for these assays. Assays for kinase function are also disclosed in U.S. Pat. Nos. 5,798,246; 5,581,784; 5,702,936, all of which are incorporated by reference for these assays. Assays are also disclosed in Houslay et al. (1997), TIBS 22:217-224, Bloom et al. (1996), Proc. Natl. Acad. Sci, USA 93:14188-14192, Zhu et al. (1997) J. Biol. Chem. 272:16152-16157, and Beavo (1995), Physiological Reviews 75:725-748, also incorporated by reference for these assays.

Binding and/or activating compounds can also be screened by using chimeric kinase proteins in which one or more domains, sites, and the like, as disclosed herein, or parts thereof, can be replaced by their heterologous counterparts derived from other kinase isoforms of the same family or from kinase isoforns of any other kinase family. For example, a catalytic region can be used that interacts with a different substrate or effector specificity and/or affinity than the native kinase. Accordingly, a different set of signal transduction or other pathway components is available as an end-point assay for activation. Alternatively, a heterologous targeting sequence can replace the native targeting sequence. This will result in different subcellular or cellular localization and accordingly can result in having an effect on a different signal transduction or other biochemical pathway. Accordingly, a different set of signal transduction or other pathway components is available as an endpoint assay for activation. As a further alternative, the site of modification by an effector protein, for example phosphorylation by a protein kinase, can be replaced with the site from a different effector protein. This could also provide the use of a different signal transduction or other pathway for endpoint determination. Activation can also be detected by a reporter gene containing an easily detectable coding region operably linked to a transcriptional regulatory sequence that is part of the native signal transduction pathway.

The invention provides competition binding assays designed to discover compounds that interact with the kinase. Thus, a compound is exposed to a kinase polypeptide under conditions that allow the compound to bind or to otherwise interact with the polypeptide. Soluble kinase polypeptide is also added to the mixture. If the test compound interacts with the soluble kinase polypeptide, it decreases the amount of complex formed or activity from the kinase target. This type of assay is particularly useful in cases in which compounds are sought that interact with specific regions of the kinase. Thus, the soluble polypeptide that competes with the target kinase region is designed to contain peptide sequences corresponding to the region of interest.

Another type of competition-binding assay can be used to discover compounds that interact with specific functional sites. As an example, an effector protein kinase and a candidate compound can be added to a sample of the kinase. Compounds that interact with the kinase at the same site as the effector protein kinase will reduce the amount of complex formed between the kinase and the effector protein kinase. Accordingly, it is possible to discover a compound that specifically prevents interaction between the kinase and the effector protein kinase. Another example involves adding a candidate compound to a sample of kinase and substrate protein. A compound that competes with substrate protein will reduce the amount of phosphorylation or binding of the substrate protein to the kinase. Accordingly, compounds can be discovered that directly interact with the kinase and compete with substrate protein. Such assays can involve any other component that interacts with the kinase.

To perform cell-free drug screening assays, it is desirable to immobilize either the kinase, or fragment, or its target molecule to facilitate separation of complexes from uncomplexed forms of one or both of the proteins, as well as to accommodate automation of the assay.

Techniques for immobilizing proteins on matrices can be used in the drug screening assays. In one embodiment, a fusion protein can be provided which adds a domain that allows the protein to be bound to a matrix. For example, glutathione-S-transferase/kinase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes is dissociated. Alternatively, the complexes can be dissociated from the matrix, separated by SDS-PAGE, and the level of kinase-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a kinase-binding target component, such as substrate or effector protein kinase, and a candidate compound are incubated in the kinase-presenting wells and the amount of complex trapped in the well can be quantitated. Methods for detecting such complexes, in addition to those described above for the GST-immobilized complexes, include immunodetection of complexes using antibodies reactive with the kinase target molecule, or which are reactive with kinase and compete with the target molecule; as well as enzyme-linked assays which rely on detecting an enzymatic activity associated with the target molecule.

Modulators of kiase level or activity identified according to these assays can be used to test the effects of modulation of expression of the enzyme on the outcome of clinically relevant disorders. This can be accomplished in vitro, in vivo, such as in human clinical trials, and in test models derived from other organisms, such as non-human transgenic subjects. Modulation in such subjects includes, but is not limited to, modulation of the cells, tissues, and disorders particularly disclosed herein. Modulators of kinase activity identified according to these drug screening assays can be used to treat a subject with a disorder mediated by the kinase pathway, by treating cells that express the kinase, such as those disclosed herein, especially in FIGS. 2-14, as well as those disorders disclosed in the references cited herein above.

In one embodiment, the cells that are treated are derived from liver, cardiovascular tissue, prostate, skeletal muscle, brain, heart, aorta, adipose, fetal kidney, fetal heart, and undifferentiated osteoblasts, and as such, modulation is particularly relevant to disorders involving these tissues. In another embodiment, modulation is in virus-infected cells and particularly virus-infected liver cells. In another embodiment modulation is in fibrotic liver, liver having a tendency to become fibrotic as a result of a preexisting condition or disorder, such as viral infection, fibrosis which is due to genetic disease, external injury or stimulus, or other physiological condition which results in or has a tendency to result in liver fibrosis. Methods to study liver fibrosis are known in the art and include but are not limited to such assays as bile duct ligation or bile duct ligation/scission assays (Lee et al. (2000) Arch. Pharm. Res. 23:613-619), and carbon tetrachloride induced fibrosis (Chen et al. (1998) Chin. Med. J. 111:779-783; Lu et al. (2000) Am. J Chin. Med. 28:361-370; Mucke et al. (2000) Int. J. Colorectal Dis. 15:335-341). Such conditions include but are not limited to alcoholic liver disease, viral hepatitis and biliary disease. In some cases fibrosis can also be caused by hereditary hematochromatosis (HHC) that results from iron overload, Wilson disease which results from copper overload, Vitamin A intoxication, and cystic fibrosis.

Expression of the kinase is detected in vascular endothelial cells, vascular smooth muscle cells, in vitro and in vivo models of angiogenesis, and in vivo atherosclerosis models. Irregular angiogenesis occurs in cancer, diabetic blindness, age-related macular degeneration, rheumatoid arthritis, and psoriasis. Thus, modulation of 20893 expression in atherosclerosis and angiogenesis disorders is relevant.

Significant expression of the kinase is also detected in fibrotic lung biopsies (data not shown). Accordingly, modulation of expression of the kinase is also particularly relevant in fibrotic lung disorders. Generally, modulation is relevant in any virus-infected tissue where such infection is associated with or directly causes fibrosis. In addition, expression is significantly decreased in differentiated osteoblasts relative to undifferentiated osteoblasts. Accordingly, modulation of the gene is also relevant in disorders involving differentiation and development of bone, including bone mass. Such disorders include, but are not limited to, osteoporosis and osteopetrosis.

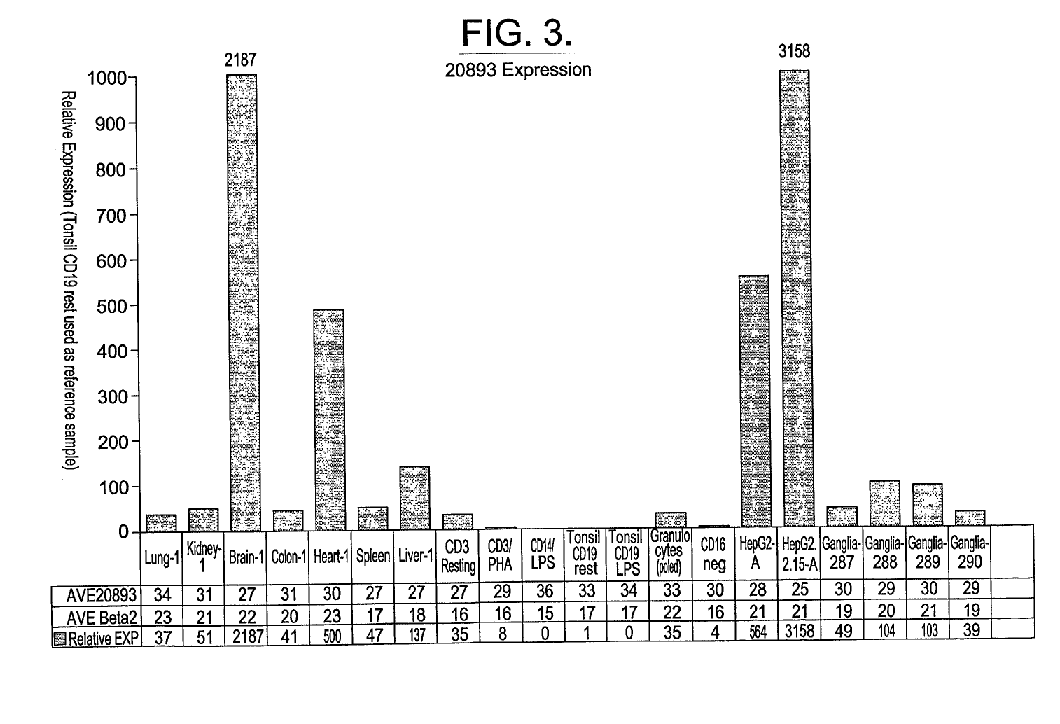

In the present case, RNA was isolated from HepG2 (immortalized human hepatocyte cells) and a HepG2 cell line stably transfected with the HBV genome. These cell lines can be used to screen for anti-HBV compounds. The RNA was labeled by synthesizing ³³P-labeled cDNA and hybridized to a gene array containing novel human genes identified by the inventors. 14171 RNA was found to be 6-fold more abundant in HBV-infected HepG2 cells than in uninfected HepG2 cells (FIG. 3).

TaqMan analysis showed a high expression of the gene in activated hepatic stellate cells and transforming growth factor β-treated normal human lung fibroblasts (NHLF), moderate but significant expression in fibrotic liver biopsies, fibrotic lung biopsies (NHBE), and low expression in normal adult liver. Increased expression of 20893 was detected in normal human lung and normal human dermal (NHDF) fibroblasts after transforming growth factor β treatment.

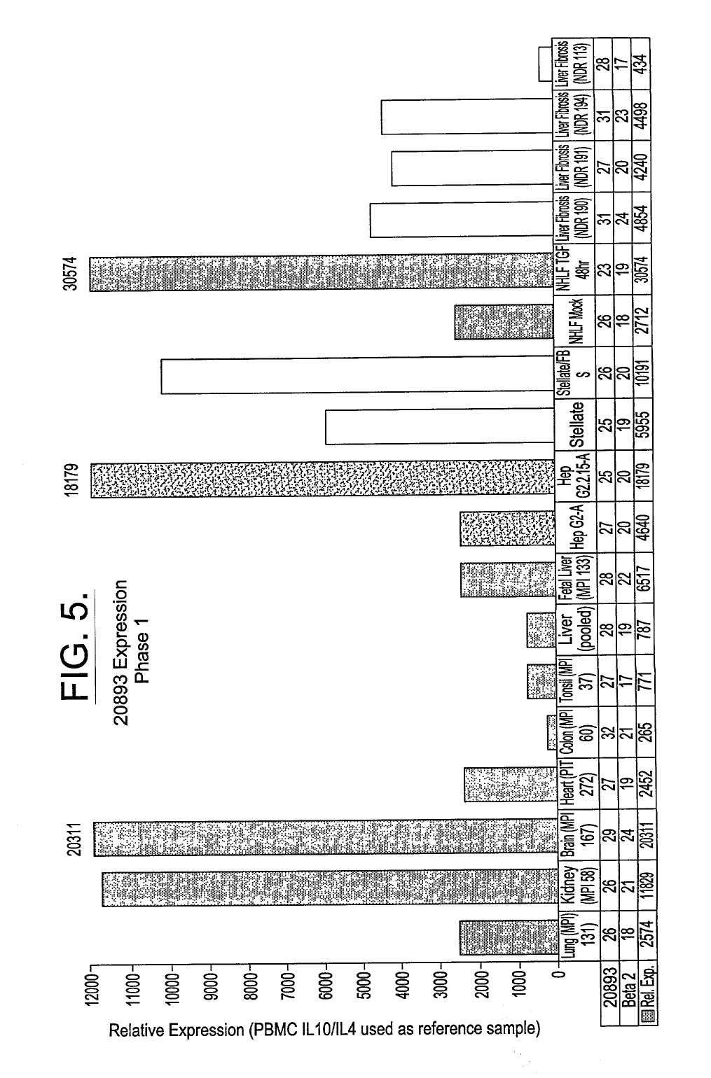

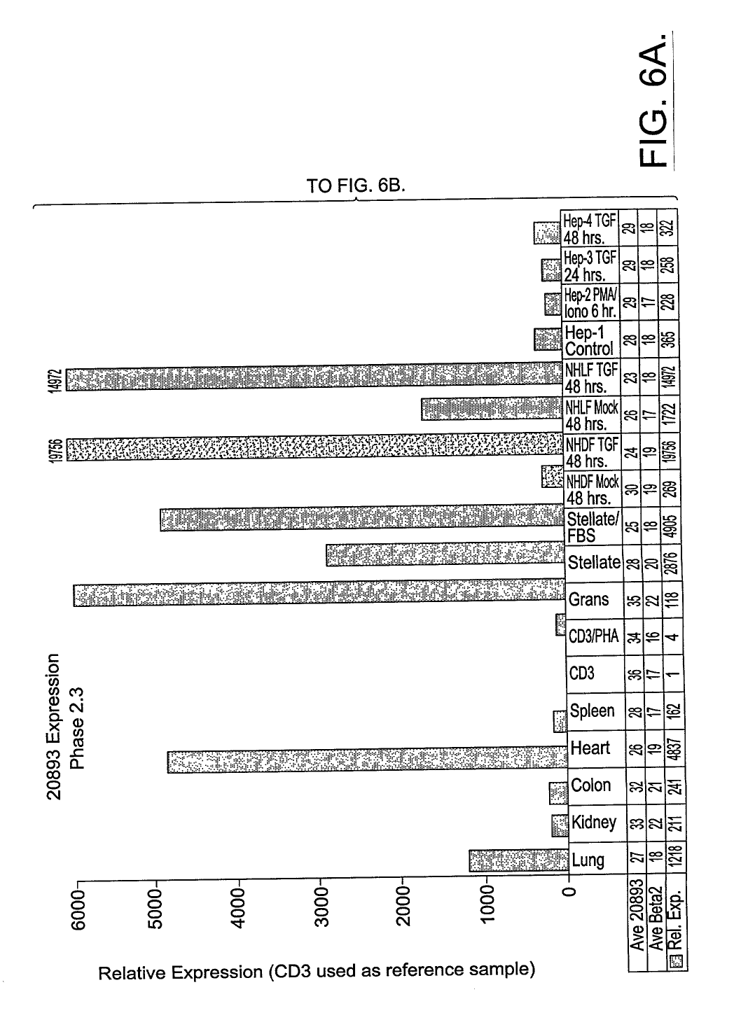

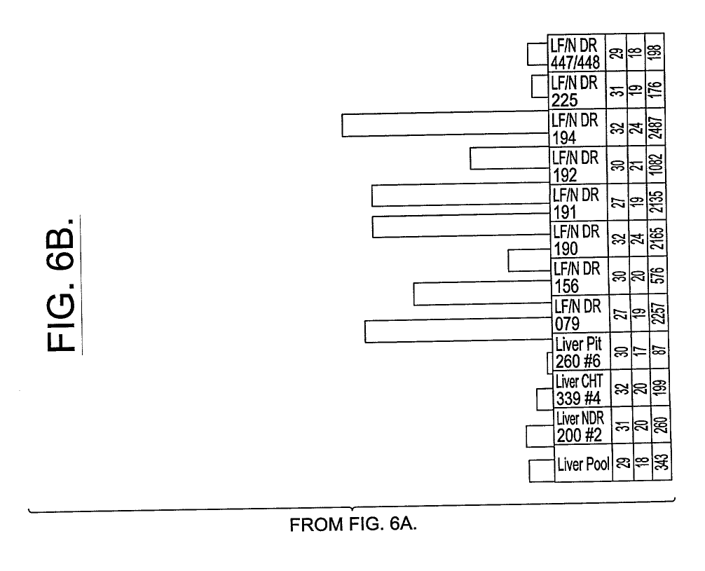

A large panel of fibrotic liver biopsies showed increased expression over several normal liver samples. High expression of 20893 was detected in activated hepatic stellate cells whereas hepatocytes showed very low levels of expression. See FIGS. 5 and 6. Hepatic stellate cells, a scarce liver type, have been proposed as an effector of the fibrotic process (Hironaka et al. (2000) Dig. Dis. Sci. 45:1935-1943). Once stimulated, stellate cells acquire the activated phenotype, proliferate and become fibrogenic. The elevated expression of 20893 in stellate cells is a significant indication for a relationship between 20893 and liver fibrosis. The disclosed invention accordingly specifically relates to methods and compositions for the modulation, diagnosis and treatment of disorders involving tissue fibrosis and particularly liver fibrosis, and more particularly fibrosis resulting from or associated with virus infection, including, but not limited to, HBV infection.

The disclosed invention also accordingly relates to methods and compositions for the modulation, diagnosis, and treatment of disorders associated with, caused by, or related to viral infection. These disorders can manifest as immune, inflammatory, respiratory, hematological, cardiovascular, and other disorders including, but not limited to, AIDS, virus associated leukemias, lymphomas, sarcomas, and carcinomas, herpetic infections and collateral symptoms, EBV infection, including mononucleosis, hepatitis virus infection, including A, B, C, and D viruses, with virally induced liver cancer, and viral pneumonias.

Viruses include, but are not limited to, those identified with carcinogenesis, including hepatitis B virus (HBV) and liver cancer, Epstein-Barr virus (EBV), and lymphoma, human T-cell lymphotrophic virus Type I (HTLV-1) and leukemia, and human Herpes virus 8 (HHV-8) and Kaposi sarcoma. Virus families to which the invention pertains include but are not limited to Adenoviridae, Picornaviridae, Coronaviridae, Orthomyxoviridae, Paramyxoviridae, Reoviridae, Caliciviridae, Hepadnaviridae, Viroid-like, Flaviviridae, Norwalk-like, Togaviridae, Parvoviridae, Poxviridae, Herpesviridae, Retroviridae, Reoviridae (Orbivirus), Arenaviridae, Bunyaviridae, Filoviridae, Hantavirus, and Papovaviridae. Respiratory diseases have been associated with Adenovirus, Echovirus, Rhinovirus, Coxsackievirus, Coronavirus, Influenza viruses A, B, Parainfluenza virus 1-4, and Respiratory syncytial virus. Viral diseases of the respiratory system include, but are not limited to, lower respiratory tract infections, conjunctivitis, diarrhea; upper respiratory tract infections, pharyngitis, rash; pleurodynia, herpangina, hand-foot-and-mouth disease; influenza, croup, bronchiolitis, and pneumonia. Digestive diseases have been associated with Mumps virus, Rotavirus, Norwalk agent, Hepatitis A Virus, Hepatitis B Virus, Hepatitis D Virus, Hepatitis C Virus, and Hepatitis E Virus. These include but are not limited to mumps, pancreatitis, orchitis; childhood diarrhea; gastroenteritis; acute viral hepatitis; acute or chronic hepatitis; with HBV, acute or chronic hepatitis; and enterically transmitted hepatitis. Systemic viral pathogens associated with skin eruptions include, but are not limited to, Measles virus, Rubella virus, Parvovirus, Vaccinia virus, Varicella-zoster virus, Herpes simplex virus 1, and Herpes simplex virus 2. Disease expression includes, but is not limited to, Measles (rubeola); German measles (rubella); Erythema infectiosum, aplastic anemia; smallpox; chickenpox, shingles; “cold sore”; and genital herpes. Systemic viral pathogens associated with hematopoietic disorders include Cytomegalovirus, Epstein-Barr virus, HTLV-I, HTLV-II, HIV-1 and HIV-2. Disease expression includes, but is not limited to, Cytomegalic inclusion disease; infectious mononucleosis; adult T-cell leukemia; tropical spastic paraparesis; and AIDS. Viral pathogens associated with Arboviral and Hemorrhagic fevers include, but are not limited, Dengue virus 1-4, yellow fever virus, Colorado tick fever virus, and regional hemorrhagic fever viruses. Disease expression includes, but is not limited to, Dengue, hemorrhagic fever; yellow fever; Colorado tick fever; Bolivian, Argentinian, Lassa fever; Crimean-Congo, Hantaan, sandfly fever; Ebola, Marburg disease; Korean, U.S. pneumonia. Viral pathogens associated with warty growths include Papillomavirus and molluscum virus. Disease expression includes, but is not limited to, Condyloma; cervical carcinoma; and molluscum contagiosum. Viral pathogens associated with diseases of the central nervous system include, but are not limited to, Poliovirus, Rabiesvirus, JC virus, and Arboviral encephalitis viruses. Disease expression includes, but is not limited to, Poliomyelitis; Rabies; progressive multifocal leukoencephalopathy (opportunistic); Eastern, Western, Venezuelan, St. Louis, Calif. group.

Disorders involving the lung include, but are not limited to, congenital anomalies; atelectasis; diseases of vascular origin, such as pulmonary congestion and edema, including hemodynamic pulmonary edema and edema caused by microvascular injury, adult respiratory distress syndrome (diffuse alveolar damage), pulmonary embolism, hemorrhage, and infarction, and pulmonary hypertension and vascular sclerosis; chronic obstructive pulmonary disease, such as emphysema, chronic bronchitis, bronchial asthma, and bronchiectasis; diffuse interstitial (infiltrative, restrictive) diseases, such as pneumoconioses, sarcoidosis, idiopathic pulmonary fibrosis, desquamative interstitial pneumonitis, hypersensitivity pneumonitis, pulmonary eosinophilia (pulmonary infiltration with eosinophilia), Bronchiolitis obliterans-organizing pneumonia, diffuse pulmonary hemorrhage syndromes, including Goodpasture syndrome, idiopathic pulmonary hemosiderosis and other hemorrhagic syndromes, pulmonary involvement in collagen vascular disorders, and pulmonary alveolar proteinosis; complications of therapies, such as drug-induced lung disease, radiation-induced lung disease, and lung transplantation; tumors, such as bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Disorders involving the colon include, but are not limited to, congenital anomalies, such as atresia and stenosis, Meckel diverticulum, congenital aganglionic megacolon-Hirschsprung disease; enterocolitis, such as diarrhea and dysentery, infectious enterocolitis, including viral gastroenteritis, bacterial enterocolitis, necrotizing enterocolitis, antibiotic-associated colitis (pseudomembranous colitis), and collagenous and lymphocytic colitis, miscellaneous intestinal inflammatory disorders, including parasites and protozoa, acquired immunodeficiency syndrome, transplantation, drug-induced intestinal injury, radiation enterocolitis, neutropenic colitis (typhlitis), and diversion colitis; idiopathic inflammatory bowel disease, such as Crohn disease and ulcerative colitis; tumors of the colon, such as non-neoplastic polyps, adenomas, familial syndromes, colorectal carcinogenesis, colorectal carcinoma, and carcinoid tumors.

Disorders involving the liver include, but are not limited to, hepatic injury; jaundice and cholestasis, such as bilirubin and bile formation; hepatic failure and cirrhosis, such as cirrhosis, portal hypertension, including ascites, portosystemic shunts, and splenomegaly; infectious disorders, such as viral hepatitis, including hepatitis A-E infection and infection by other hepatitis viruses, clinicopathologic syndromes, such as the carrier state, asymptomatic infection, acute viral hepatitis, chronic viral hepatitis, and fulminant hepatitis; autoimmune hepatitis; drug- and toxin-induced liver disease, such as alcoholic liver disease; inborn errors of metabolism and pediatric liver disease, such as hemochromatosis, Wilson disease, a₁-antitrypsin deficiency, and neonatal hepatitis; intrahepatic biliary tract disease, such as secondary biliary cirrhosis, primary biliary cirrhosis, primary sclerosing cholangitis, and anomalies of the biliary tree; circulatory disorders, such as impaired blood flow into the liver, including hepatic artery compromise and portal vein obstruction and thrombosis, impaired blood flow through the liver, including passive congestion and centrilobular necrosis and peliosis hepatis, hepatic vein outflow obstruction, including hepatic vein thrombosis (Budd-Chiari syndrome) and veno-occlusive disease; hepatic disease associated with pregnancy, such as preeclampsia and eclampsia, acute fatty liver of pregnancy, and intrehepatic cholestasis of pregnancy; hepatic complications of organ or bone marrow transplantation, such as drug toxicity after bone marrow transplantation, graft-versus-host disease and liver rejection, and nonimmunologic damage to liver allografts; tumors and tumorous conditions, such as nodular hyperplasias, adenomas, and malignant tumors, including primary carcinoma of the liver and metastatic tumors.

Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

Disorders involving the heart, include but are not limited to, heart failure, including but not limited to, cardiac hypertrophy, left-sided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcific aortic stenosis, calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroelastoma, rhabdomyoma, and sarcoma, and cardiac effects of noncardiac neoplasms; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia, and disorders involving cardiac transplantation.

Disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, disorders of angiogenesis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

Disorders involving the thymus include developmental disorders, such as DiGeorge syndrome with thymic hypoplasia or aplasia; thymic cysts; thymic hypoplasia, which involves the appearance of lymphoid follicles within the thymus, creating thymic follicular hyperplasia; and thymomas, including germ cell tumors, lynphomas, Hodgkin disease, and carcinoids. Thymomas can include benign or encapsulated thymoma, and malignant thymoma Type I (invasive thymoma) or Type II, designated thymic carcinoma.

Disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schonlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypemephroma, adenocarcinoma of kidney), which includes urothelial carcinomas of renal pelvis.

Disorders of the breast include, but are not limited to, disorders of development; inflammations, including but not limited to, acute mastitis, periductal mastitis, periductal mastitis (recurrent subareolar abscess, squamous metaplasia of lactiferous ducts), mammary duct ectasia, fat necrosis, granulomatous mastitis, and pathologies associated with silicone breast implants; fibrocystic changes; proliferative breast disease including, but not limited to, epithelial hyperplasia, sclerosing adenosis, and small duct papillomas; tumors including, but not limited to, stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, no special type, invasive lobular carcinoma, medullary carcinoma, colloid (mucinous) carcinoma, tubular carcinoma, and invasive papillary carcinoma, and miscellaneous malignant neoplasms.

Disorders in the male breast include, but are not limited to, gynecomastia and carcinoma.

Disorders involving the testis and epididymis include, but are not limited to, congenital anomalies such as cryptorchidism, regressive changes such as atrophy, inflammations such as nonspecific epididymitis and orchitis, granulomatous (autoimmune) orchitis, and specific inflammations including, but not limited to, gonorrhea, mumps, tuberculosis, and syphilis, vascular disturbances including torsion, testicular tumors including germ cell tumors that include, but are not limited to, seminoma, spermatocytic seminoma, embryonal carcinoma, yolk sac tumor choriocarcinoma, teratoma, and mixed tumors, tumore of sex cord-gonadal stroma including, but not limited to, leydig (interstitial) cell tumors and sertoli cell tumors (androblastoma), and testicular lymphoma, and miscellaneous lesions of tunica vaginalis.

Disorders involving the prostate include, but are not limited to, inflammations, benign enlargement, for example, nodular hyperplasia (benign prostatic hypertrophy or hyperplasia), and tumors such as carcinoma.

Disorders involving the thyroid include, but are not limited to, hyperthyroidism; hypothyroidism including, but not limited to, cretinism and myxedema; thyroiditis including, but not limited to, hashimoto thyroiditis, subacute (granulomatous) thyroiditis, and subacute lymphocytic (painless) thyroiditis; Graves disease; diffuse and multinodular goiter including, but not limited to, diffuse nontoxic (simple) goiter and multinodular goiter; neoplasms of the thyroid including, but not limited to, adenomas, other benign tumors, and carcinomas, which include, but are not limited to, papillary carcinoma, follicular carcinoma, medullary carcinoma, and anaplastic carcinoma; and cogenital anomalies.

Disorders involving the skeletal muscle include tumors such as rhabdomyosarcoma.

Disorders involving the ovary include, for example, polycystic ovarian disease, Stein-leventhal syndrome, Pseudomyxoma peritonei and stromal hyperthecosis; ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecoma-fibromas, androblastomas, hill cell tumors, and gonadoblastoma; and metastatic tumors such as Krukenberg tumors.

Bone-forming cells include the osteoprogenitor cells, osteoblasts, and osteocytes. The disorders of the bone are complex because they may have an impact on the skeleton during any of its stages of development. Hence, the disorders may have variable manifestations and may involve one, multiple or all bones of the body. Such disorders include, congenital malformations, achondroplasia and thanatophoric dwarfism, diseases associated with abnormal matix such as type 1 collagen disease, osteoporosis, Paget disease, rickets, osteomalacia, high-turnover osteodystrophy, low-turnover of aplastic disease, osteonecrosis, pyogenic osteomyelitis, tuberculous osteomyelitism, osteoma, osteoid osteoma, osteoblastoma, osteosarcoma, osteochondroma, chondromas, chondroblastoma, chondromyxoid fibroma, chondrosarcoma, fibrous cortical defects, fibrous dysplasia, fibrosarcoma, malignant fibrous histiocytoma, Ewing sarcoma, primitive neuroectodermal tumor, giant cell tumor, and metastatic tumors.

The invention thus provides methods for treating a disorder characterized by aberrant expression or activity of a kinase. These methods of treatment include the steps of administering the modulators of kinase activity in a pharmaceutical composition as described herein, to a subject in need of such treatment. In one embodiment, the method involves administering an agent (e.g., an agent identified by a screening assay described herein), or combination of agents that modulates (e.g., upregulates or downregulates) expression or activity of the protein. In another embodiment, the method involves administering the kinase as therapy to compensate for reduced or aberrant expression or activity of the protein.

Methods for treatment include but are not limited to the use of soluble kinase or fragments of the kinase protein that compete for ATP, GTP, AMP or GMP, effector protein or other macromolecule or substrate. These kinases or fragments can have a higher affinity for the target so as to provide effective competition.

Stimulation of activity is desirable in situations in which the protein is abnormally downregulated and/or in which increased activity is likely to have a beneficial effect. Likewise, inhibition of activity is desirable in situations in which the protein is abnormally upregulated and/or in which decreased activity is likely to have a beneficial effect. In one example of such a situation, a subject has a disorder characterized by aberrant development or cellular differentiation. In another example, the subject has a proliferative disease (e.g., cancer) or a disorder characterized by an aberrant hematopoietic response. In another example, it is desirable to achieve tissue regeneration in a subject (e.g., where a subject has undergone brain or spinal cord injury and it is desirable to regenerate neuronal tissue in a regulated manner).

The invention also provides methods for diagnosing a disease or predisposition to disease mediated by the kinase, including, but not limited to, diseases involving tissues in which the kinases are expressed, as disclosed herein, and particularly in skeletal muscle, brain, heart, fetal kidney, fetal heart, osteoblast tissue, virus-infected tissue and especially where virus infection results in tissue fibrosis, for example in lung and liver, and fibrotic tissues. In view of these results, in one embodiment of the invention, these disorders are treated by modulating the level or activity of the kinase gene in diseased hearts. Since expression has been shown virally-infected liver cells as well as in fibrotic lung and liver samples, treatment is especially directed to these cells. Likewise, in one embodiment, diagnosis is directed to cells and tissues involved in these disorders. As mentioned above, treatment and diagnosis can be in human subjects in which the disease normally occurs and in model systems, both in vitro and in vivo, such as in transgenic animals.

Accordingly, methods are directed to detecting the presence, or levels of, the kinase in a cell, tissue, or organism. The methods involve contacting a biological sample with a compound capable of interacting with the kinase such that the interaction can be detected.

One agent for detecting kinase is an antibody capable of selectively binding to kinase. A biological sample includes tissues, cells and biological fluids isolated from a subject, as well as tissues, cells and fluids present within a subject.

The invention also provides methods for diagnosing active disease, or predisposition to disease, in a patient having a variant kinase. Thus, kinase can be isolated from a biological sample and assayed for the presence of a genetic mutation that results in an aberrant protein. This includes amino acid substitution, deletion, insertion, rearrangement, (as the result of aberrant splicing events), and inappropriate post-translational modification. Analytic methods include altered electrophoretic mobility, altered tryptic peptide digest, altered kinase activity in cell-based or cell-free assay, alteration in ATP, GTP, AMP or GMP binding or activation, effector molecule (e.g., protein) binding or phosphorylation, or antibody-binding pattern, substrate binding or phosphorylation, altered isoelectric point, direct amino acid sequencing, and any other of the known assay techniques useful for detecting mutations in a protein in general or in a kinase specifically.

In vitro techniques for detection of kinase include enzyme linked immunosorbent assays (ELISAs), Western blots, immunoprecipitations and immunofluorescence. Alternatively, the protein can be detected in vivo in a subject by introducing into the subject a labeled anti-kinase antibody. For example, the antibody can be labeled with a radioactive marker whose presence and location in a subject can be detected by standard imaging techniques. Particularly useful are methods, which detect the allelic variant of the kinase expressed in a subject, and methods, which detect fragments of the kinase in a sample.

The invention also provides methods of pharmacogenomic analysis including, but not limited to, in the cells, tissues and disorders disclosed herein in which expression of the kinase either occurs or shows differential expression. Pharmacogenomics deal with clinically significant hereditary variations in the response to drugs due to altered drug disposition and abnormal action in affected persons. See, e.g., Eichelbaum, M. (1996) Clin. Exp. Pharmacol. Physiol. 23(10-11):983-985, and Linder, M. W. (1997) Clin. Chem. 43(2):254-266. The clinical outcomes of these variations result in severe toxicity of therapeutic drugs in certain individuals or therapeutic failure of drugs in certain individuals as a result of individual variation in metabolism. Thus, the genotype of the individual can determine the way a therapeutic compound acts on the body or the way the body metabolizes the compound. Further, the activity of drug metabolizing enzymes affects both the intensity and duration of drug action. Thus, the pharmacogenomics of the individual permit the selection of effective compounds and effective dosages of such compounds for prophylactic or therapeutic treatment based on the individual's genotype. The discovery of genetic polymorphisms in some drug metabolizing enzymes has explained why some patients do not obtain the expected drug effects, show an exaggerated drug effect, or experience serious toxicity from standard drug dosages. Polymorphisms can be expressed in the phenotype of the extensive metabolizer and the phenotype of the poor metabolizer. Accordingly, genetic polymorphism may lead to allelic protein variants of the kinase in which one or more of the kinase functions in one population is different from those in another population. The polypeptides can be used as a target to ascertain a genetic predisposition that can affect treatment modality. Thus, in a substrate protein-based treatment, polymorphism may give rise to catalytic regions that are more or less active. Accordingly, dosage would necessarily be modified to maximize the therapeutic effect within a given population containing the polymorphism. As an alternative to genotyping, specific polymorphic polypeptides could be identified.

The invention also provides for monitoring therapeutic effects during clinical trials and other treatment. Thus, the therapeutic effectiveness of an agent that is designed to increase or decrease gene expression, protein levels or kinase activity can be monitored over the course of treatment using the kinase polypeptides as an end-point target. The monitoring can be, for example, as follows: (i) obtaining a pre-administration sample from a subject prior to administration of the agent; (ii) detecting the level of expression or activity of the protein in the pre-administration sample; (iii) obtaining one or more post-administration samples from the subject; (iv) detecting the level of expression or activity of the protein in the post-administration samples; (v) comparing the level of expression or activity of the protein in the pre-administration sample with the protein in the post-administration sample or samples; and (vi) increasing or decreasing the administration of the agent to the subject accordingly.

Polypeptides

The methods and uses herein disclosed can be based on polypeptide reagents and targets. The invention is thus based on the use of a human protein kinase. Specifically, an expressed sequence tag (EST) was selected based on homology to protein kinase sequences. This EST was used to design primers based on sequences that it contains and used to identify a cDNA from a human cDNA library. Positive clones were sequenced and the overlapping fragments were assembled. Analysis of the assembled sequence revealed that the cloned cDNA molecule encodes a protein kinase homologous to (among others) GenBank human sequence AB011109 and rat 5′ AMP activated protein kinase α-1 catalytic subunit U40819.

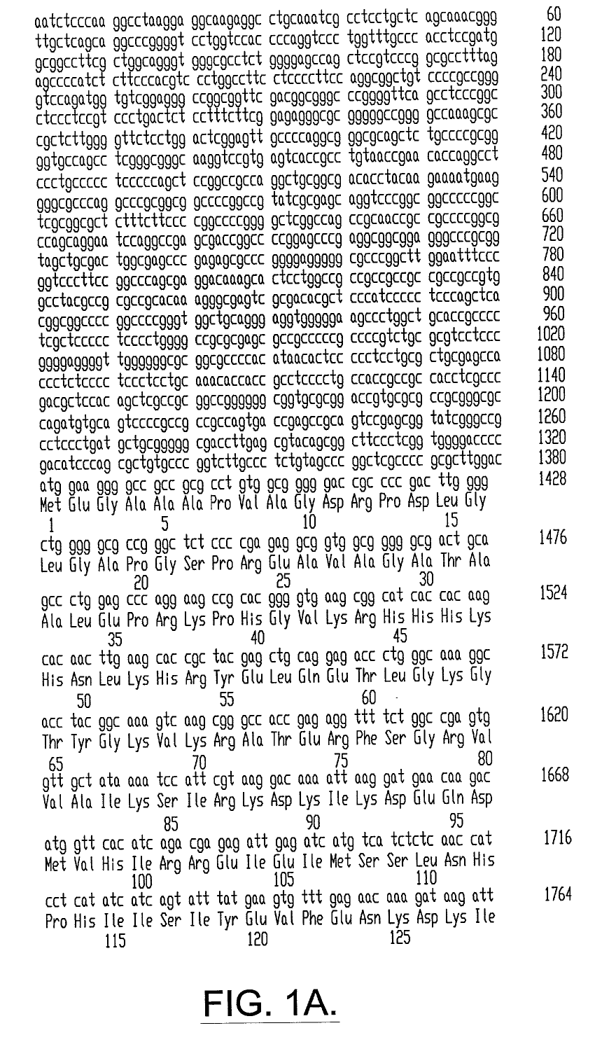

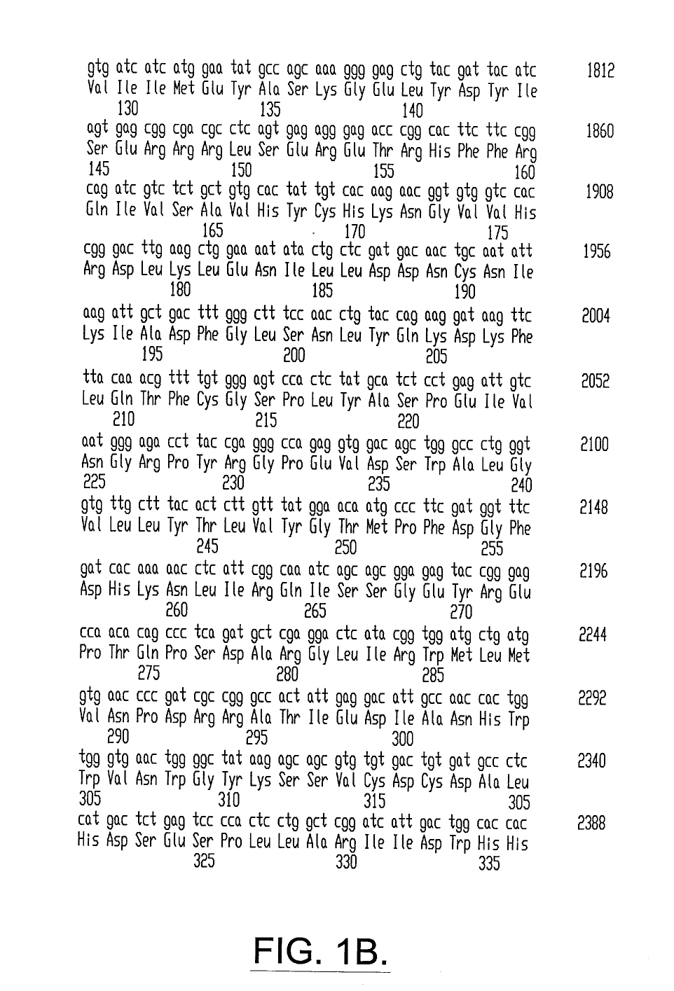

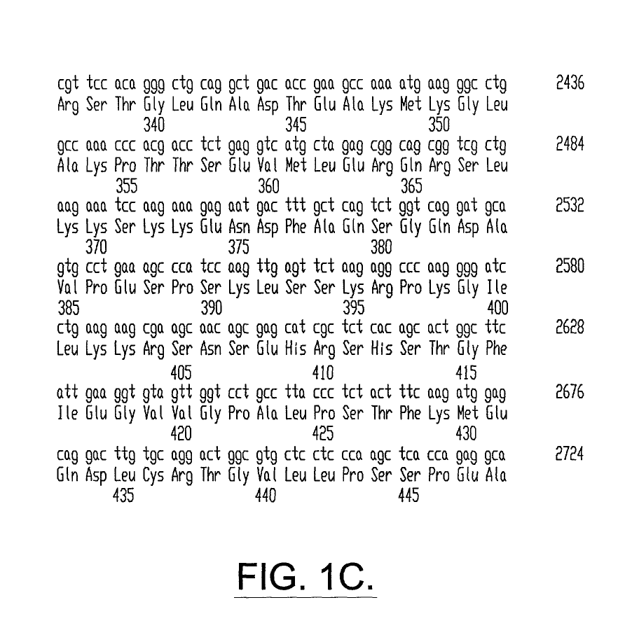

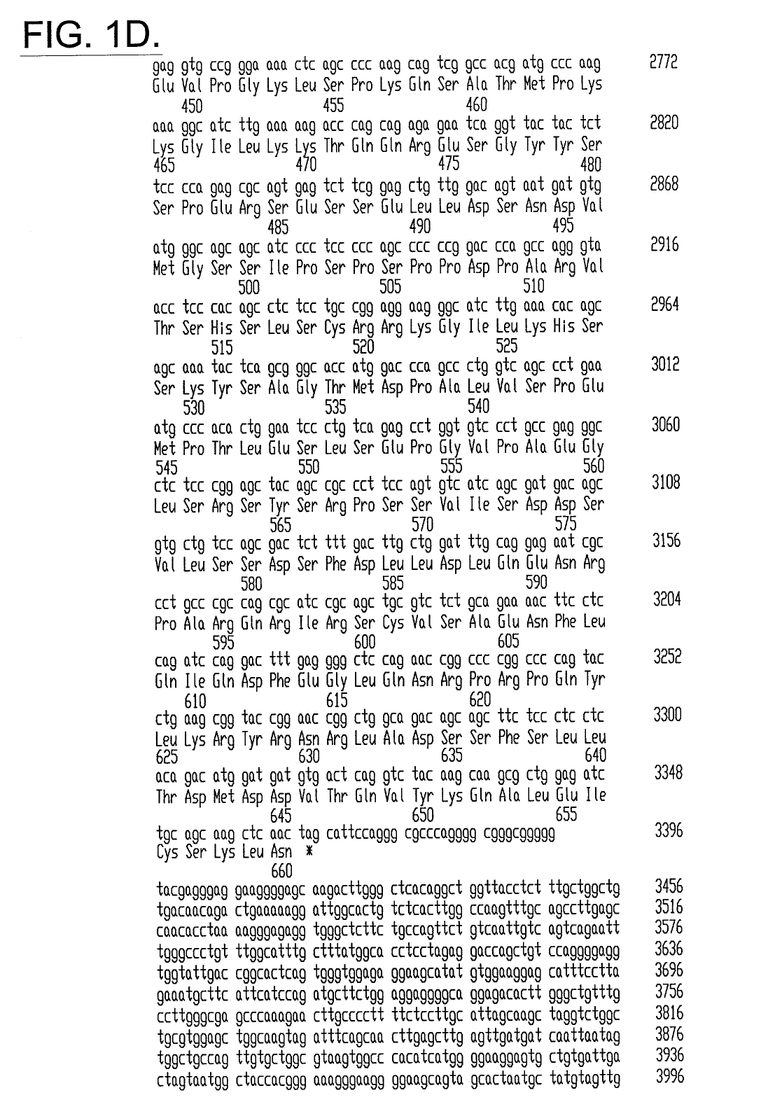

The invention thus relates to expression of a protein kinase having the deduced amino acid sequence shown in FIG. 1 (SEQ ID NO:2).

“Kinase polypeptide,” “kinase protein,” “protein kinase polypeptide,” “protein kinase protein,” and “protein kinase” all refer to the polypeptide in SEQ ID NO:2. The terms, however, further include the numerous variants described herein, as well as fragments derived from the full-length kinases and variants. By “variants” is intended proteins or polypeptides having an amino acid sequence that is at least about 60%, 65%, or 70%, preferably about 75%, 85%, 95%, or 98% identical to the amino acid sequence of SEQ ID NO:2. Variants also include polypeptides encoded by the cDNA insert of the plasmid deposited with the ATCC as Patent Deposit No. PTA-2201, or polypeptides encoded by a nucleic acid molecule that hybridizes to the nucleic acid molecule of SEQ ID NO:3 or a complement thereof, under stringent conditions. Variants retain the biological activity (e.g. the protein kinase activity) of the reference polypeptide set forth in SEQ ID NO:2. In another embodiment, a variant of an isolated polypeptide of the present invention differs, by at least 1, but less than 5, 10, 20, 50, or 100 amino acid residues from the sequence shown in SEQ ID NO:2. If alignment is needed for this comparison the sequences should be aligned for maximum identity. “Looped” out sequences from deletions or insertions, or mismatches, are considered differences. Variants include polypeptides that differ in amino acid sequence due to natural allelic variation or mutagenesis.

Tissues and/or cells in which the kinase is found include, but are not limited to those shown in FIGS. 2-14, and particularly in liver, particularly fibrotic liver, kidney, skeletal muscle, brain, heart, fetal heart. Expression is high in hepatic stellate cells. In addition, the kinase is expressed in fibrotic tissues such as liver and lung. In addition, the kinase is expressed in virally-infected cells, specifically liver cells.

The present invention thus utilizes an isolated or purified kinase polypeptide and variants and fragments thereof.

As used herein, a polypeptide is said to be “isolated” or “purified” when it is substantially free of cellular material, when it is isolated from recombinant and non- recombinant cells, or free of chemical precursors or other chemicals when it is chemically synthesized. A polypeptide, however, can be joined to another polypeptide with which it is not normally associated in a cell and still be considered “isolated” or “purified.”

The kinase polypeptides can be purified to homogeneity. It is understood, however, that preparations in which the polypeptide is not purified to homogeneity are useful and considered to contain an isolated form of the polypeptide. The critical feature is that the preparation allows for the desired function of the polypeptide, even in the presence of considerable amounts of other components. Thus, the invention encompasses various degrees of purity.

In one embodiment, the language “substantially free of cellular material” includes preparations of the kinase having less than about 30% (by dry weight) other proteins (i.e., contaminating protein), less than about 20% other proteins, less than about 10% other proteins, or less than about 5% other proteins. When the polypeptide is recombinantly produced, it can also be substantially free of culture medium, i.e., culture medium represents less than about 20%, less than about 10%, or less than about 5% of the volume of the protein preparation.

A kinase polypeptide is also considered to be isolated when it is part of a membrane preparation or is purified and then reconstituted with membrane vesicles or liposomes.

The language “substantially free of chemical precursors or other chemicals” includes preparations of the kinase polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, the kinase polypeptide comprises the amino acid sequence shown in SEQ ID NO:2. However, the invention also encompasses sequence variants. Variants include a substantially homologous protein encoded by the same genetic locus in an organism, i.e., an allelic variant.

Variants also encompass proteins derived from other genetic loci in an organism, but having substantial homology to the kinase of SEQ ID NO:2. Variants also include proteins substantially homologous to the kinase but derived from another organism, i.e., an ortholog. Variants also include proteins that are substantially homologous to the kinase that are produced by chemical synthesis. Variants also include proteins that are substantially homologous to the kinase that are produced by recombinant methods. It is understood, however, that variants exclude any amino acid sequences disclosed prior to the invention.

As used herein, two proteins (or a region of the proteins) are substantially homologous when the amino acid sequences are at least about 70-75%, typically at least about 80-85%, and most typically at least about 90-95% or more homologous. A substantially homologous amino acid sequence, according to the present invention, will be encoded by a nucleic acid sequence hybridizing to the nucleic acid sequence, or portion thereof, of the sequence shown in SEQ ID NO:1 under stringent conditions as more fully described below.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% or more of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). For example, amino acid or nucleotide sequences that contain a common structural domain having at least about 60%, 65%, 70%, 75%, 80%, 85%, 90%, 95%, 96%, 97%, 98%, or 99% identity are defined herein as identical. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (1970) J. Mol. Biol. 48:444-453 algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) is using a Blossum 62 scoring matrix with a gap open penalty of 12, a gap extend penalty of 4, and a frameshift gap penalty of 5.

The percent identity between two amino acid or nucleotide sequences can be determined using the algorithm of E. Meyers and W. Miller (1989) CABIOS 4:11-17 which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 20893 nucleic acid molecules of the invention. BLAST protein searches can be performed with the XBLAST program, score=50, wordlength=3 to obtain amino acid sequences homologous to 20893 protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also encompasses polypeptides having a lower degree of identity but having sufficient similarity so as to perform one or more of the same functions performed by the kinase. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al., Science 247:1306-1310 (1990).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

A variant polypeptide can differ in amino acid sequence by one or more substitutions, deletions, insertions, inversions, fusions, and truncations or a combination of any of these.

Variant polypeptides can be fully functional or can lack function in one or more activities. Thus, in the present case, variations can affect the function, for example, of one or more of the regions corresponding to a catalytic region, regulatory region, targeting region, region involved in membrane association, region involved in enzyme activation, for example, by phosphorylation, and regions involved in interaction with components of a phosphorylation-dependent signal transduction or other pathway.

Fully functional variants typically contain only conservative variation or variation in non-critical residues or in non-critical regions. Functional variants can also contain substitution of similar amino acids, which results in no change or an insignificant change in function. Alternatively, such substitutions may positively or negatively affect function to some degree.

Non-functional variants typically contain one or more non-conservative amino acid substitutions, deletions, insertions, inversions, or truncation or a substitution, insertion, inversion, or deletion in a critical residue or critical region.

As indicated, variants can be naturally-occurring or can be made by recombinant means or chemical synthesis to provide useful and novel characteristics for the kinase polypeptide. This includes preventing immunogenicity from pharmaceutical formulations by preventing protein aggregation.

Useful variations further include alteration of catalytic activity. For example, one embodiment involves a variation at the binding site that results in binding but not phosphorylation, or slower phosphorylation, of substrate. A further useful variation at the same site can result in altered affinity for substrate. Useful variation includes one that prevents autophosphorylation or activation by AMP or by an effector protein kinase or other effector. Another useful variation provides a fusion protein in which one or more domains or subregions are operationally fused to one or more domains or subregions from another kinase isoform or family.

Amino acids that are essential for function can be identified by methods known in the art, such as site-directed mutagenesis or alanine-scanning mutagenesis (Cunningham et al. (1985) Science 244:1081-1085). The latter procedure introduces single alanine mutations at every residue in the molecule. The resulting mutant molecules are then tested for biological activity, such as substrate phosphorylation in vitro or effector molecule, such as a protein (for example kinase)-dependent in vitro activity, such as proliferative activity. Sites that are critical for effector protein or other effector molecule binding, AMP or GMP binding or activation, effector activation, substrate binding and phosphorylation, etc. can also be determined by structural analysis such as crystallization, nuclear magnetic resonance or photoaffinity labeling (Smith et al. (1992) J. Mol. Biol. 224:899-904; de Vos et al. (1992) Science 255:306-312).

Substantial homology can be to the entire nucleic acid or amino acid sequence or to fragments of these sequences. Generally, nucleic acid molecules that are fragments of 20893 protein kinase comprise 10, 20, 30, 40, 50, 60, 70, 80, 90, 100, 150, 200, 250, 300, 350, 400, 450, 500, 550, 600, 650, 700, 750, 800, 850, 900, 950, 1000, 1050, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200,5300, 5400, 5500, 5600, 5700, 5800, 5900, 6000, 6100, 6200, 6300, 6400, 6500, 6600, 6700, 6800, or up to 6828 nucleotides of SEQ ID NO:1. Alternatively, a nucleic acid molecule that is a fragment of a nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400,4400-4500, 4500-4600, 4600-4700, 4700-4800, 4800-4900, 4900-5000, 5000-5100, 5100-5200, 5200-5300, 5300-5400, 5400-5500, 5500-5600, 5600-5700, 5700-5800, 5800-5900, 5900-6000, 6000-6100, 6100-6200, 6200-6300, 6300-6400, 6400-6500, 6500-6600, 6600-6700, 6700-6800, 6800-6828 of SEQ ID NO:1. The sequence listed in SEQ ID NO:5 encompasses approximately nucleotides 1357 to 4241 of SEQ ID NO:1.

The invention thus also includes polypeptide fragments of the kinase. Fragments can be derived from the amino acid sequence shown in SEQ ID NO:2. However, the invention also encompasses fragments of the variants of the kinase as described herein. SEQ ID NO:6 encompasses amino acid sequence encoded by SEQ ID NO:5.

Accordingly, a fragment can comprise at least about 10, 15, 20, 25, 30, 35, 40, 45, 50, 60, 70, 80, 90, 100, 150,200, 250, 300, 350, 400, 450, 500, 550, 600, 650, or 661 contiguous amino acids disclosed in SEQ ID NO:2. Fragments can retain one or more of the biological activities of the protein, for example the ability to bind to or phosphorylate a (e.g., protein) substrate, as well as fragments that can be used as an immunogen to generate kinase antibodies.

Biologically active fragments (peptides which are, for example, 5, 7, 10, 12, 15, 20, 30, 35, 36, 37, 38, 39, 40, 50, 100, 150, 200, 250, 300, 350, 400, 450, 500, 550, 600, 650, or 661 or more amino acids in length) can comprise a domain or motif, e.g., catalytic site, kinase signature, and sites for glycosylation, protein kinase C phosphorylation, casein kinase II phosphorylation, tyrosine kinase phosphorylation, and N-myristoylation. Further possible fragments include a catalytic site or domain, an allosteric binding site, sites important for cellular and subcellular targeting, sites functional for interacting with components of other cGMP or cAMP-dependent signal transduction or other biochemical pathways, and regulatory sites.

Such domains or motifs can be identified by means of routine computerized homology searching procedures.

Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived.

These regions can be identified by well-known methods involving computerized homology analysis.

The invention also provides fragments with immunogenic properties. These contain an epitope-bearing portion of the kinase and variants. These epitope-bearing peptides are useful to raise antibodies that bind specifically to a kinase polypeptide or region or fragment. These peptides can contain at least 10, 12, at least 14, or between at least about 15 to about 30 amino acids.

Non-limiting examples of antigenic polypeptides that can be used to generate antibodies include but are not limited to peptides derived from an extracellular site. However, intracellularly-made antibodies (“intrabodies”) are also encompassed, which would recognize intracellular peptide regions.

The epitope-bearing kinase polypeptides may be produced by any conventional means (Houghten, R. A. (1985) Proc. Natl. Acad. Sci. USA 82:5131-5135). Simultaneous multiple peptide synthesis is described in U.S. Pat. No. 4,631,211.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the kinase fragment and an additional region fused to the carboxyl terminus of the fragment.

The invention thus provides chimeric or fusion proteins. These comprise a kinase peptide sequence operatively linked to a heterologous peptide having an amino acid sequence not substantially homologous to the kinase. “Operatively linked” indicates that the kinase peptide and the heterologous peptide are fused in-frame. The heterologous peptide can be fused to the N-terminus or C-terminus of the kinase or can be internally located.

In one embodiment the fusion protein does not affect kinase function per se. For example, the fusion protein can be a GST-fusion protein in which the kinase sequences are fused to the N- or C-terminus of the GST sequences. Other types of fusion proteins include, but are not limited to, enzymatic fusion proteins, for example beta-galactosidase fusions, yeast two-hybrid GAL-4 fusions, poly-His fusions and Ig fusions. Such fusion proteins, particularly poly-His fusions, can facilitate the purification of recombinant kinase. In certain host cells (e.g., mammalian host cells), expression and/or secretion of a protein can be increased by using a heterologous signal sequence. Therefore, in another embodiment, the fusion protein contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists (Bennett et al. (1995) J. Mol. Recog. 8:52-58 (1995) and Johanson et al. J. Biol. Chem. 270:9459-9471). Thus, this invention also utilizes soluble fusion proteins containing a kinase polypeptide and various portions of the constant regions of heavy or light chains of immunoglobulins of various subclass (IgG, IgM, IgA, IgE). Preferred as immunoglobulin is the constant part of the heavy chain of human IgG, particularly IgG1, where fusion takes place at the hinge region. For some uses it is desirable to remove the Fc after the fusion protein has been used for its intended purpose, for example when the fusion protein is to be used as antigen for immunizations. In a particular embodiment, the Fc part can be removed in a simple way by a cleavage sequence, which is also incorporated and can be cleaved with factor Xa.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al. (1992) Current Protocols in Molecular Biology). Moreover, many expression vectors are commercially available that already encode a fusion moiety (e.g., a GST protein). A kinase-encoding nucleic acid can be cloned into such an expression vector such that the fusion moiety is linked in-frame to the kinase.

Another form of fusion protein is one that directly affects kinase functions. Accordingly, a kinase polypeptide is encompassed by the present invention in which one or more of the kinase domains (or parts thereof) has been replaced by homologous domains (or parts thereof) from another kinase family. Accordingly, various permutations are possible. For example, the aminoterninal regulatory domain, or subregion thereof, can be replaced with the domain or subregion from another isoform or kinase family. As a further example, a catalytic domain or parts thereof, can be replaced. Thus, chimeric kinases can be formed in which one or more of the native domains or subregions has been replaced by another.

Additionally, chimeric kinase proteins can be produced in which one or more functional sites is derived from a different isoform, or from another kinase family. It is understood, however, that sites could be derived from kinase families that occur in the mammalian genome but which have not yet been discovered or characterized. Such sites include but are not limited to a catalytic site, regulatory site, site important for targeting to subcellular and cellular locations, site functional for interaction with components of a phosphorylation-dependent signal transduction or other pathway, effector phosphorylation site, glycosylation sites, and other functional sites such as are disclosed herein.

The isolated kinases can be purified from cells that naturally express it, such as from those shown in FIGS. 2-14 and/or specifically disclosed herein above, among others, especially purified from cells that have been altered to express it (recombinant), or synthesized using known protein synthesis methods.

In one embodiment, the protein is produced by recombinant DNA techniques. For example, a nucleic acid molecule encoding the kinase polypeptide is cloned into an expression vector, the expression vector introduced into a host cell and the protein expressed in the host cell. The protein can then be isolated from the cells by an appropriate purification scheme using standard protein purification techniques.

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd ed., T. E. Creighton, W.H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (1990) Meth. Enzymol. 182: 626-646) and Rattan et al. (1992) Ann. N. Y. Acad. Sci. 663:48-62).

As is also well known, polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the aminoterminal residue of polypeptides made in E. coli, prior to proteolytic processing, almost invariably will be N- formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

The same type of modification may be present in the same or varying degree at several sites in a given polypeptide. Also, a given polypeptide may contain more than one type of modification.

Methods of Using Antibodies

Methods for using antibodies as disclosed herein are particularly applicable to the cells, tissues and disorders shown in FIGS. 2-14 and as otherwise discussed herein above.

The invention provides methods using antibodies that selectively bind to the kinase and its variants and fragments. An antibody is considered to selectively bind, even if it also binds to other proteins that are not substantially homologous with the kinase. These other proteins share homology with a fragment or domain of the kinase. This conservation in specific regions gives rise to antibodies that bind to both proteins by virtue of the homologous sequence. In this case, it would be understood that antibody binding to the kinase is still selective.

The invention provides methods of using antibodies to isolate a kinase by standard techniques, such as affinity chromatography or immunoprecipitation. The antibodies can facilitate the purification of the kinase from cells naturally expressing it and cells recombinantly producing it.

The antibodies can be used to detect the presence of kinase in cells or tissues to determine the pattern of expression of the kinase among various tissues in an organism and over the course of normal development.

The antibodies can be used to detect kinase in situ, in vitro, or in a cell lysate or supernatant in order to evaluate the abundance and pattern of expression.

The antibodies can be used to assess abnormal tissue distribution or abnormal expression during development.

Antibody detection of circulating fragments of the fall length kinase can be used to identify kinase turnover.

Further, the antibodies can be used to assess kinase expression in disease states such as in active stages of the disease or in an individual with a predisposition toward disease related to kinase function. When a disorder is caused by an inappropriate tissue distribution, developmental expression, or level of expression of the kinase protein, the antibody can be prepared against the normal kinase protein. If a disorder is characterized by a specific mutation in the kinase, antibodies specific for this mutant protein can be used to assay for the presence of the specific mutant kinase. However, intracellularly-made antibodies (“intrabodies”) are also encompassed, which would recognize intracellular kinase peptide regions.

The antibodies can also be used to assess normal and aberrant subcellular localization in cells in the various tissues in an organism. Antibodies can be developed against the whole kinase or portions of the kinase.

The diagnostic uses can be applied, not only in genetic testing, but also in monitoring a treatment modality. Accordingly, where treatment is ultimately aimed at correcting kinase expression level or the presence of aberrant kinases and aberrant tissue distribution or developmental expression, antibodies directed against the kinase or relevant fragments can be used to monitor therapeutic efficacy.

Antibodies accordingly can be used diagnostically to monitor protein levels in tissue as part of a clinical testing procedure, e.g., to, for example, determine the efficacy of a given treatment regimen.

Additionally, antibodies are useful in pharmacogenomic analysis. Thus, antibodies prepared against polymorphic kinase can be used to identify individuals that require modified treatment modalities.

Antibodies can also be used in diagnostic procedures as an immunological marker for aberrant kinase analyzed by electrophoretic mobility, isoelectric point, tryptic peptide digest, and other physical assays known to those in the art.

The antibodies are also useful for tissue typing. Thus, where the kinase is expressed in a specific tissue, antibodies that are specific for this kinase can be used to identify the tissue type.

The antibodies are also useful for inhibiting kinase function, for example, blocking binding of ATP, GTP, GMP or AMP, effector protein, substrate, or the catalytic site.

These uses can also be applied in a therapeutic context in which treatment involves inhibiting kinase function. Antibodies can be prepared against specific fragments containing sites required for function or against intact kinase.

Completely human antibodies are particularly desirable for therapeutic treatment of human patients. For an overview of this technology for producing human antibodies, see Lonberg et al. (1995) Int. Rev. Immunol. 13:65-93. For a detailed discussion of this technology for producing human antibodies and human monoclonal antibodies and protocols for producing such antibodies, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806.

The invention also encompasses kits for using antibodies to detect the presence of a kinase protein in a biological sample. The kit can comprise antibodies such as a labeled or labelable antibody and a compound or agent for detecting kinase in a biological sample; means for determining the amount of kinase in the sample; and means for comparing the amount of kinase in the sample with a standard. The compound or agent can be packaged in a suitable container. The kit can further comprise instructions for using the kit to detect kinase.

Antibodies

The methods for using antibodies described above are based on the generation of antibodies that specifically bind to the kinase or its variants or fragments.

To generate antibodies, an isolated kinase polypeptide is used as an immunogen to generate antibodies using standard techniques for polyclonal and monoclonal antibody preparation. Either the full-length protein or antigenic peptide fragment can be used.

Antibodies are preferably prepared from these regions or from discrete fragments in these regions. However, antibodies can be prepared from any region of the peptide as described herein. A preferred fragment produces an antibody that diminishes or completely prevents effector protein phosphorylation or binding or substrate phosphorylation or binding. Antibodies can be developed against the entire kinase or domains of the kinase as described herein. Antibodies can also be developed against specific functional sites as disclosed herein.

The antigenic peptide can comprise a contiguous sequence of at least 12, 14, 15, or 30 amino acid residues. In one embodiment, fragments correspond to regions that are located on the surface of the protein, e.g., hydrophilic regions. These fragments are not to be construed, however, as encompassing any fragments, which may be disclosed prior to the invention.

Antibodies can be polyclonal or monoclonal. An intact antibody, or a fragment thereof (e.g. Fab or F(ab′)₂) can be used.

Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

An appropriate immunogenic preparation can be derived from native, recombinantly expressed, or chemically synthesized peptides.

Methods for Using the Polynucleotide

The methods and uses described herein below for the kinase polynucleotide are particularly applicable to the cells, tissues, and disorders shown in FIGS. 2-14, and specifically discussed herein above.

The nucleic acid fragments useful to practice the invention provide probes or primers in assays, such as those described herein. “Probes” are oligonucleotides that hybridize in a base-specific manner to a complementary strand of nucleic acid. Such probes include polypeptide nucleic acids, as described in Nielsen et al. (1991) Science 254:1497-1500. Typically, a probe comprises a region of nucleotide sequence that hybridizes under highly stringent conditions to at least about 15, typically about 20-25, and more typically about 40, 50 or 75 consecutive nucleotides of the nucleic acid sequence shown in SEQ ID NO:1; SEQ ID NO:3, or the complements thereof. More typically, the probe further comprises a label, e.g., radioisotope, fluorescent compound, enzyme, or enzyme co-factor.

As used herein, the term “primer” refers to a single-stranded oligonucleotide which acts as a point of initiation of template-directed DNA synthesis using well-known methods (e.g., PCR, LCR) including, but not limited to those described herein. The appropriate length of the primer depends on the particular use, but typically ranges from about 15 to 30 nucleotides. The term “primer site” refers to the area of the target DNA to which a primer hybridizes. The term “primer pair” refers to a set of primers including a 5′ (upstream) primer that hybridizes with the 5′ end of the nucleic acid sequence to be amplified and a 3′ (downstream) primer that hybridizes with the complement of the sequence to be amplified.

The kinase polynucleotides can be utilized as probes and primers in biological assays.

Where the polynucleotides are used to assess kinase properties or functions, such as in the assays described herein, all or less than all of the entire cDNA can be useful. Assays specifically directed to kinase functions, such as assessing agonist or antagonist activity, encompass the use of known fragments. Further, diagnostic methods for assessing kinase function can also be practiced with any fragment, including those fragments that may have been known prior to the invention. Similarly, in methods involving treatment of kinase dysfunction, all fragments are encompassed including those, which may have been known in the art.

The invention utilizes the kinase polynucleotides as a hybridization probe for cDNA and genomic DNA to isolate a full-length cDNA and genomic clones encoding variant polypeptides and to isolate cDNA and genomic clones that correspond to variants producing the same polypeptides shown in SEQ ID NO:2 or the other variants described herein. This method is useful for isolating variant genes and cDNA that are expressed in the cells, tissues, and disorders disclosed herein.

The probe can correspond to any sequence along the entire length of the gene encoding the kinase. Accordingly, it could be derived from 5′ noncoding regions, the coding region, and 3′ noncoding regions.

The nucleic acid probe can be, for example, the full-length cDNA of SEQ ID NO:1, or a fragment thereof, such as an oligonucleotide of at least 12, 15, 30, 50, 100, 250 or 500 nucleotides in length and sufficient to specifically hybridize under stringent conditions to mRNA or DNA.

Fragments of the polynucleotides can also be used to synthesize larger fragments or full-length polynucleotides described herein. For example, a fragment can be hybridized to any portion of an mRNA and a larger or full-length cDNA can be produced.

Fragments can also be used to synthesize antisense molecules of desired length and sequence.

Antisense nucleic acids, useful in treatment and diagnosis, can be designed using the nucleotide sequences of SEQ ID NO:1 or SEQ ID NO:3, and constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6- isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

Additionally, the nucleic acid molecules useful to practice the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670. PNAs can be further modified, e.g., to enhance their stability, specificity or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra, Finn et al. (1996) Nucleic Acids Res. 24(17):3357-63, Mag et al. (1989) Nucleic Acids Res. 17:5973, and Peterser et al. (1975) Bioorganic Med. Chem. Lett. 5:1119.

The nucleic acid molecules and fragments useful to practice the invention can also include other appended groups such as peptides (e.g., for targeting host cell kinases in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/0918) or the blood brain barrier (see, e.g., PCT Publication No. WO 89/10134). In addition, oligonucleotides can be modified with hybridization-triggered cleavage agents (see, e.g., Krol et al. (1988) Bio-Techniques 6:958-976) or intercalating agents (see, e.g., Zon (1988) Pharm Res. 5:539-549).

The kinase polynucleotides can also be used as primers for PCR to amplify any given region of a kinase polynucleotide.

The kinase polynucleotides can also be used to construct recombinant vectors. Such vectors include expression vectors that express a portion of, or all of, the kinase polypeptides. Vectors also include insertion vectors, used to integrate into another polynucleotide sequence, such as into the cellular genome, to alter in situ expression of kinase genes and gene products. For example, an endogenous kinase coding sequence can be replaced via homologous recombination with all or part of the coding region containing one or more specifically introduced mutations.

The kinase polynucleotides can also be used to express antigenic portions of the kinase protein.

The kinase polynucleotides can also be used as probes for determining the chromosomal positions of the kinase polynucleotides by means of in situ hybridization methods, such as FISH. (For a review of this technique, see Verma et al. (1988) Human Chromosomes: A Manual of Basic Techniques (Pergamon Press, New York), and PCR mapping of somatic cell hybrids. The mapping of the sequence to chromosomes is important in correlating these sequences with genes associated with disease, especially where translocations and/or amplification has occurred. .

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease mapped to the same chromosomal region, can then be identified through linkage analysis (co-inheritance of physically adjacent genes), described in, for example, Egeland et al. ((1987) Nature 325:783-787).

Moreover, differences in the DNA sequences between individuals affected and unaffected with a disease associated with a specified gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations, that are visible from chromosome spreads, or detectable using PCR based on that DNA sequence. Ultimately, complete sequencing of genes from several individuals can be performed to confirm the presence of a mutation and to distinguish mutations from polymorphisms.

The kinase polynucleotide probes can also be used to determine patterns of the presence of the gene encoding the kinase with respect to tissue distribution, for example, whether gene duplication has occurred and whether the duplication occurs in all or only a subset of cells in a tissue. The genes can be naturally occurring or can have been introduced into a cell, tissue, or organism exogenously.

The kinase polynucleotides can also be used to design ribozymes corresponding to all, or a part, of the mRNA produced from genes encoding the polynucleotides described herein, the ribozymes being useful to treat or diagnose a disorder or otherwise modulate expression of the nucleic acid.

The kinase polynucleotides can also be used to make vectors that express part, or all, of the kinase polypeptides.

The kinase polynucleotides can also be used to construct host cells expressing a part, or all, of the kinase polynucleotides and polypeptides.

The kinase polynucleotides can also be used to construct transgenic animals expressing all, or a part, of the kinase polynucleotides and polypeptides.

The kinase polynucleotides can also be used as hybridization probes to determine the level of kinase nucleic acid expression. Accordingly, the probes can be used to detect the presence of, or to determine levels of, kinase nucleic acid in cells, tissues, and in organisms. DNA or RNA level can be determined. Probes can be used to assess gene copy number in a given cell, tissue, or organism. This is particularly relevant in cases in which there has been an amplification of the kinase gene.

Alternatively, the probe can be used in an in situ hybridization context to assess the position of extra copies of the kinase gene, as on extrachromosomal elements or as integrated into chromosomes in which the kinase gene is not normally found, for example, as a homogeneously staining region.

These uses are relevant for diagnosis of disorders involving an increase or decrease in kinase expression relative to normal, such as a proliferative disorder, a differentiative or developmental disorder, or a hematopoietic disorder, such as in the cells and tissues shown in FIGS. 2-14 and otherwise specifically discussed herein. Thus in one embodiment, disorders include viral infections and diseases involving fibrotic tissue.

Thus, the present invention provides a method for identifying a disease or disorder associated with aberrant expression or activity of kinase nucleic acid, in which a test sample is obtained from a subject and nucleic acid (e.g., mRNA, genomic DNA) is detected, wherein the presence of the nucleic acid is diagnostic for a subject having or at risk of developing a disease or disorder associated with aberrant expression or activity of the nucleic acid.

One aspect of the invention relates to diagnostic assays for determining nucleic acid expression as well as activity in the context of a biological sample (e.g., blood, serum, cells, tissue) to determine whether an individual has a disease or disorder, or is at risk of developing a disease or disorder, associated with aberrant nucleic acid expression or activity. Such assays can be used for prognostic or predictive purpose to thereby prophylactically treat an individual prior to the onset of a disorder characterized by or associated with expression or activity of the nucleic acid molecules.

In vitro techniques for detection of mRNA include Northern hybridizations and in situ hybridizations. In vitro techniques for detecting DNA includes Southern hybridizations and in situ hybridization.

Probes can be used as a part of a diagnostic test kit for identifying cells or tissues that express the kinase, such as by measuring the level of a kinase-encoding nucleic acid in a sample of cells from a subject e.g., mRNA or genomic DNA, or determining if the kinase gene has been mutated.

Nucleic acid expression assays are useful for drug screening to identify compounds that modulate kinase nucleic acid expression (e.g., antisense, polypeptides, peptidomimetics, small molecules or other drugs). A cell is contacted with a candidate compound and the expression of mRNA determined. The level of expression of the mRNA in the presence of the candidate compound is compared to the level of expression of the MRNA in the absence of the candidate compound. The candidate compound can then be identified as a modulator of nucleic acid expression based on this comparison and be used, for example to treat a disorder characterized by aberrant nucleic acid expression. The modulator can bind to the nucleic acid or indirectly modulate expression, such as by interacting with other cellular components that affect nucleic acid expression.

Modulatory methods can be performed in vitro (e.g., by culturing the cell with the agent) or, alternatively, in vivo (e.g., by administering the gene to a subject) in patients or in transgenic animals.

The invention thus provides a method for identifying a compound that can be used to treat a disorder associated with expression of the kinase gene. The method typically includes assaying the ability of the compound to modulate the expression of the kinase nucleic acid and thus identifying a compound that can be used to treat a disorder characterized by excessive or deficient kinase nucleic acid expression.

The assays can be performed in cell-based and cell-free systems, such as systems using the tissues described herein, in which the gene is expressed or in model systems for the disorders to which the invention pertains. Cell-based assays include cells naturally expressing the kinase nucleic acid or recombinant cells genetically engineered to express specific nucleic acid sequences.

Alternatively, candidate compounds can be assayed in vivo in patients or in transgenic animals.

The assay for kinase nucleic acid expression can involve direct assay of nucleic acid levels, such as mRNA levels, or on collateral compounds involved in the signal or other pathway (such as phosphorylated substrate). Further, the expression of genes that are up- or down-regulated in response to the kinase signal pathway can also be assayed. In this embodiment the regulatory regions of these genes can be operably linked to a reporter gene such as luciferase.

Thus, modulators of kinase gene expression can be identified in a method wherein a cell is contacted with a candidate compound and the expression of mRNA determined. The level of expression of kinase mRNA in the presence of the candidate compound is compared to the level of expression of kinase mRNA in the absence of the candidate compound. The candidate compound can then be identified as a modulator of nucleic acid expression based on this comparison and be used, for example to treat a disorder characterized by aberrant nucleic acid expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

Accordingly, the invention provides methods of treatment, with the nucleic acid as a target, using a compound identified through drug screening as a gene modulator to modulate kinase nucleic acid expression. Modulation includes both up-regulation (i.e. activation or agonization) or down-regulation (suppression or antagonization) or effects on nucleic acid activity (e.g. when nucleic acid is mutated or improperly modified). Treatment is of disorders characterized by aberrant expression or activity of the nucleic acid.

The gene is particularly relevant for the treatment of disorders involving the tissues shown in FIGS. 2-14, and discussed herein.

Alternatively, a modulator for kinase nucleic acid expression can be a small molecule or drug identified using the screening assays described herein as long as the drug or small molecule inhibits the kinase nucleic acid expression.

The kinase polynucleotides are also useful for monitoring the effectiveness of modulating compounds on the expression or activity of the kinase gene in clinical trials or in a treatment regimen. Thus, the gene expression pattern can serve as a barometer for the continuing effectiveness of treatment with the compound, particularly with compounds to which a patient can develop resistance. The gene expression pattern can also serve as a marker indicative of a physiological response of the affected cells to the compound. Accordingly, such monitoring would allow either increased administration of the compound or the administration of alternative compounds to which the patient has not become resistant. Similarly, if the level of nucleic acid expression falls below a desirable level, administration of the compound could be commensurately decreased.

Monitoring can be, for example, as follows: (i) obtaining a pre-administration sample from a subject prior to administration of the agent; (ii) detecting the level of expression of a specified mRNA or genomic DNA of the invention in the pre-administration sample; (iii) obtaining one or more post-administration samples from the subject; (iv) detecting the level of expression or activity of the mRNA or genomic DNA in the post-administration samples; (v) comparing the level of expression or activity of the mRNA or genomic DNA in the pre-administration sample with the mRNA or genomic DNA in the post-administration sample or samples; and (vi) increasing or decreasing the administration of the agent to the subject accordingly.

The kinase polynucleotides can be used in diagnostic assays for qualitative changes in kinase nucleic acid, and particularly in qualitative changes that lead to pathology. The polynucleotides can be used to detect mutations in kinase genes and gene expression products such as mRNA. The polynucleotides can be used as hybridization probes to detect naturally-occurring genetic mutations in the kinase gene and thereby to determine whether a subject with the mutation is at risk for a disorder caused by the mutation. Mutations include deletion, addition, or substitution of one or more nucleotides in the gene, chromosomal rearrangement, such as inversion or transposition, modification of genomic DNA, such as aberrant methylation patterns or changes in gene copy number, such as amplification. Detection of a mutated form of the kinase gene associated with a dysfunction provides a diagnostic tool for an active disease or susceptibility to disease when the disease results from overexpression, underexpression, or altered expression of a kinase.

Mutations in the kinase gene can be detected at the nucleic acid level by a variety of techniques. Genomic DNA can be analyzed directly or can be amplified by using PCR prior to analysis. RNA or cDNA can be used in the same way.

In certain embodiments, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a gene under conditions such that hybridization and amplification of the gene (if present) occurs, and detecting the presence or absence of an amplification product, or detecting the size of the amplification product and comparing the length to a control sample. Deletions and insertions can be detected by a change in size of the amplified product compared to the normal genotype. Point mutations can be identified by hybridizing amplified DNA to normal RNA or antisense DNA sequences.

It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

Alternatively, mutations in a kinase gene can be directly identified, for example, by alterations in restriction enzyme digestion patterns determined by gel electrophoresis.

Further, sequence-specific ribozymes (U.S. Pat. No. 5,498,531) can be used to score for the presence of specific mutations by development or loss of a ribozyme cleavage site.

Perfectly matched sequences can be distinguished from mismatched sequences by nuclease cleavage digestion assays or by differences in melting temperature.

Sequence changes at specific locations can also be assessed by nuclease protection assays such as RNase and S1 protection or the chemical cleavage method.

Furthermore, sequence differences between a mutant kinase gene and a wild-type gene can be determined by direct DNA sequencing. A variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the gene include methods in which protection from cleavage agents is used to detect mismatched bases in RNA/RNA or RNA/DNA duplexes (Myers et al. (1985) Science 230:1242); Cotton et al. (1988) PNAS 30 85:4397; Saleeba et al. (1992) Meth. Enzymol. 21 7:286-295), electrophoretic mobility of mutant and wild type nucleic acid is compared (Orita et al. (1989) PNAS 86:2766; Cotton et al. (1993) Mutat. Res. 285:125-144; and Hayashi et al. (1992) Genet. Anal. Tech. Appl. 9:73-79), and movement of mutant or wild-type fragments in polyacrylamide gels containing a gradient of denaturant is assayed using denaturing gradient gel electrophoresis (Myers et al. (1985) Nature 313:495). The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5). Examples of other techniques for detecting point mutations include, selective oligonucleotide hybridization, selective amplification, and selective primer extension.

In other embodiments, genetic mutations can be identified by hybridizing a sample and control nucleic acids, e.g., DNA or RNA, to high density arrays containing hundreds or thousands of oligonucleotide probes (Cronin et al. (1996) Human Mutation 7:244-255; Kozal et al. (1996) Nature Medicine 2:753-759). For example, genetic mutations can be identified in two dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild- type gene and the other complementary to the mutant gene.

The kinase polynucleotides can also be used for testing an individual for a genotype that while not necessarily causing the disease, nevertheless affects the treatment modality. Thus, the polynucleotides can be used to study the relationship between an individual's genotype and the individual's response to a compound used for treatment (pharmacogenomic relationship). In the present case, for example, a mutation in the kinase gene that results in altered affinity for substrate could result in an excessive or decreased drug effect with standard concentrations of a substrate-based treatment. Accordingly, the kinase polynucleotides described herein can be used to assess the mutation content of the gene in an individual in order to select an appropriate compound or dosage regimen for treatment.

Thus polynucleotides displaying genetic variations that affect treatment provide a diagnostic target that can be used to tailor treatment in an individual. Accordingly, the production of recombinant cells and animals containing these polymorphisms allow effective clinical design of treatment compounds and dosage regimens.

The methods can involve obtaining a control biological sample from a control subject, contacting the control sample with a compound or agent capable of detecting mRNA, or genomic DNA, such that the presence of mRNA or genomic DNA is detected in the biological sample, and comparing the presence of mRNA or genomic DNA in the control sample with the presence of mRNA or genomic DNA in the test sample. “Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

The kinase polynucleotides can further be used to provide polynucleotide reagents, e.g., labeled or labelable probes which can be used in, for example, an in situ hybridization technique, to identify a specific tissue. This is useful in cases in which a forensic pathologist is presented with a tissue of unknown origin. Panels of kinase probes can be used to identify tissue by species and/or by organ type.

In a similar fashion, these primers and probes can be used to screen tissue culture for contamination (i.e. screen for the presence of a mixture of different types of cells in a culture).

Alternatively, the kinase polynucleotides can be used directly to block transcription or translation of kinase gene sequences by means of antisense or ribozyme constructs. Thus, in a disorder characterized by abnormally high or undesirable kinase gene expression, nucleic acids can be directly used for treatment.

The kinase polynucleotides are thus useful as antisense constructs to control kinase gene expression in cells, tissues, and organisms. A DNA antisense polynucleotide is designed to be complementary to a region of the gene involved in transcription, preventing transcription and hence production of kinase protein. An antisense RNA or DNA polynucleotide would hybridize to the mRNA and thus block translation of mRNA into kinase protein.

Examples of antisense molecules useful to inhibit nucleic acid expression include antisense molecules complementary to a fragment of the 5′ untranslated region of SEQ ID NO:1 which also includes the start codon and antisense molecules which are complementary to a fragment of the 3′ untranslated region of SEQ ID NO:1.

Alternatively, a class of antisense molecules can be used to inactivate mRNA in order to decrease expression of kinase nucleic acid. Accordingly, these molecules can treat a disorder characterized by abnormal or undesired kinase nucleic acid expression. This technique involves cleavage by means of ribozymes containing nucleotide sequences complementary to one or more regions in the mRNA that attenuate the ability of the mRNA to be translated. Possible regions include coding regions and particularly coding regions corresponding to the catalytic and other functional activities of the kinase protein.

The kinase polynucleotides also provide vectors for gene therapy in patients containing cells that are aberrant in kinase gene expression. Thus, recombinant cells, which include the patient's cells that have been engineered ex vivo and returned to the patient, are introduced into an individual where the cells produce the desired kinase protein to treat the individual.

The invention also encompasses kits for detecting the presence of a kinase nucleic acid in a biological sample. For example, the kit can comprise reagents such as a labeled or labelable nucleic acid or agent capable of detecting kinase nucleic acid in a biological sample; means for determining the amount of kinase nucleic acid in the sample; and means for comparing the amount of kinase nucleic acid in the sample with a standard. The compound or agent can be packaged in a suitable container. The kit can further comprise instructions for using the kit to detect kinase mRNA or DNA.

Polynucleotides

The methods and uses described herein can be based on the kinase polynucleotide as a reagent or as a target. The invention thus provides methods and uses for the nucleotide sequence in SEQ ID NO: 1. The specifically disclosed cDNA comprises the coding region and 5′ and 3′ untranslated sequences in SEQ ID NO:1.

The invention provides isolated polynucleotides encoding the kinase. The term “kinase polynucleotide,” “kinase nucleic acid,” “protein kinase polynucleotide” and “protein kinase nucleic acid” all refer to the sequences shown in SEQ ID NO: 1 or SEQ ID NO:3 or in the deposited cDNAs. The term “kinase polynucleotide” or “kinase nucleic acid” further includes variants and fragments of the kinase polynucleotides.

An “isolated” kinase nucleic acid is one that is separated from other nucleic acid present in the natural source of the kinase nucleic acid. Preferably, an “isolated” nucleic acid is free of sequences which naturally flank the kinase nucleic acid (i.e., sequences located at the 5′ and 3′ ends of the nucleic acid) in the genomic DNA of the organism from which the nucleic acid is derived. However, there can be some flanking nucleotide sequences, for example up to about 5 KB. The important point is that the kinase nucleic acid is isolated from flanking sequences such that it can be subjected to the specific manipulations described herein, such as recombinant expression, preparation of probes and primers, and other uses specific to the kinase nucleic acid sequences.

Moreover, an “isolated” nucleic acid molecule, such as a cDNA or RNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid comprises at least about 50, 80 or 90% (on a molar basis) of all macromolecular species present.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

In some instances, the isolated material will form part of a composition (or example, a crude extract containing other substances), buffer system or reagent mix. In other circumstances, the material may be purified to essential homogeneity, for example as determined by PAGE or column chromatography such as HPLC. Preferably, an isolated nucleic acid comprises at least about 50, 80 or 90% (on a molar basis) of all macromolecular species present.

The kinase polynucleotides can encode the mature protein plus additional amino or carboxyterminal amino acids, or amino acids interior to the mature polypeptide (when the mature form has more than one polypeptide chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

The kinase polynucleotides include, but are not limited to, the sequence encoding the mature polypeptide alone, the sequence encoding the mature polypeptide and additional coding sequences, such as a leader or secretory sequence (e.g., a pre-pro or pro-protein sequence), the sequence encoding the mature polypeptide, with or without the additional coding sequences, plus additional non-coding sequences, for example introns and non- coding 5′ and 3′ sequences such as transcribed but non-translated sequences that play a role in transcription, mRNA processing (including splicing and polyadenylation signals), ribosome binding and stability of mRNA. In addition, the polynucleotide may be fused to a marker sequence encoding, for example, a peptide that facilitates purification.

Kinase polynucleotides can be in the form of RNA, such as mRNA, or in the form DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

In one embodiment, the kinase nucleic acid comprises only the coding region.

The invention further provides variant kinase polynucleotides, and fragments thereof, that differ from the nucleotide sequence shown in SEQ ID NO:1 due to degeneracy of the genetic code and thus encode the same protein as that encoded by the nucleotide sequence shown in SEQ ID NO:1.

The invention also provides kinase nucleic acid molecules encoding the variant polypeptides described herein. Such polynucleotides may be naturally occurring, such as allelic variants (same locus), homologs (different locus), and orthologs (different organism), or may be constructed by recombinant DNA methods or by chemical synthesis. Such non-naturally occurring variants may be made by mutagenesis techniques, including those applied to polynucleotides, cells, or organisms. Accordingly, as discussed above, the variants can contain nucleotide substitutions, deletions, inversions and insertions.

Typically, variants have a substantial identity with a nucleic acid molecule of SEQ ID NO:1, SEQ ID NO:3, or the complements thereof. Variation can occur in either or both the coding and non-coding regions. The variations can produce both conservative and non-conservative amino acid substitutions.

Orthologs, homologs, and allelic variants can be identified using methods well known in the art. Generally, nucleotide sequence variants of the invention will have at least about 60%, 65%, 70%, 75%, 80%, 85%, 90%, 91%,92%, 93%, 94%, 95%,96%, 97%, 98%, or 99% identity to the nucleotide sequence shown in SEQ ID NO:1 or a fragment of this sequence. Such nucleic acid molecules can readily be identified as being able to hybridize under stringent conditions, to the nucleotide sequence shown in SEQ ID NO:1 or a fragment of the sequence. It is understood that stringent hybridization does not indicate substantial homology where it is due to general homology, such as poly A sequences.

As used herein, the term “hybridizes under stringent conditions” describes conditions for hybridization and washing. Stringent conditions are known to those skilled in the art and can be found in Current Protocols in Molecular Biology John Wiley & Sons, N.Y. (1989), 6.3.1-6.3.6. Aqueous and nonaqueous methods are described in that reference and either can be used. A preferred, example of stringent hybridization conditions are hybridization in 6×sodium chloride/sodium citrate (SSC) at about 45° C., followed by one or more washes in 0.2×SSC, 0.1% SDS at 50° C. Another example of stringent hybridization conditions are hybridization in 6×sodium chloride/sodium citrate (SSC) at about 45° C., followed by one or more washes in 0.2×SSC, 0.1% SDS at 55° C. A further example of stringent hybridization conditions are hybridization in 6×sodium chloride/sodium citrate (SSC) at about 45° C., followed by one or more washes in 0.2×SSC, 0.1% SDS at 60° C. Preferably, stringent hybridization conditions are hybridization in 6×sodium chloride/sodium citrate (SSC) at about 45° C., followed by one or more washes in 0.2×SSC, 0.1% SDS at 65° C. Particularly preferred stringency conditions (and the conditions that should be used if the practitioner is uncertain about what conditions should be applied to determine if a molecule is within a hybridization limitation of the invention) are 0.5M Sodium Phosphate, 7% SDS at 65° C., followed by one or more washes at 0.2×SSC, 1% SDS at 65° C. Preferably, an isolated nucleic acid molecule of the invention that hybridizes under stringent conditions to the sequence of SEQ ID NO:1 or SEQ ID NO:2, corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

As understood by those of ordinary skill, the exact conditions can be determined empirically and depend on ionic strength, temperature and the concentration of destabilizing agents such as formamide or denaturing agents such as SDS. Other factors considered in determining the desired hybridization conditions include the length of the nucleic acid sequences, base composition, percent mismatch between the hybridizing sequences and the frequency of occurrence of subsets of the sequences within other non-identical sequences. Thus, equivalent conditions can be determined by varying one or more of these parameters while maintaining a similar degree of identity or similarity between the two nucleic acid molecules.

The present invention also provides isolated nucleic acids that contain a single or double stranded fragment or portion that hybridizes under stringent conditions to the nucleotide sequence of SEQ ID NO:1 or the complement of SEQ ID NO:1. In one embodiment, the nucleic acid consists of a portion of the nucleotide sequence of SEQ ID NO:1 and the complement of SEQ ID NO:1. The nucleic acid fragments of the invention are at least about 15, preferably at least about 18, 20, 23 or 25 nucleotides, and can be 30, 40, 50, 100, 200, 500 or more nucleotides in length. Longer fragments, for example, 30 or more nucleotides in length, which encode antigenic proteins or polypeptides described herein are useful.

Furthermore, the invention provides polynucleotides that comprise a fragment of the full-length kinase polynucleotide. The fragment can be single or double-stranded and can comprise DNA or RNA. The fragment can be derived from either the coding or the non-coding sequence.

In another embodiment an isolated kinase nucleic acid encodes the entire coding region. In another embodiment the isolated kinase nucleic acid encodes a sequence corresponding to the mature protein that may be from about amino acid 6 to the last amino acid. Other fragments include nucleotide sequences encoding the amino acid fragments described herein.

Thus, kinase nucleic acid fragments further include sequences corresponding to the domains described herein, subregions also described, and specific functional sites. Kinase nucleic acid fragments also include combinations of the domains, segments, and other functional sites described above. A person of ordinary skill in the art would be aware of the many permutations that are possible.

Where the location of the domains or sites have been predicted by computer analysis, one of ordinary skill would appreciate that the amino acid residues constituting these domains can vary depending on the criteria used to define the domains.

However, it is understood that a kinase fragment includes any nucleic acid sequence that does not include the entire gene.

The invention also provides kinase nucleic acid fragments that encode epitope bearing regions of the kinase proteins described herein.

Methods Using Vectors and Host Cells

The methods using vectors and host cells are particularly relevant where vectors are expressed in the cells, tissues, and disorders shown in FIGS. 2-14, and otherwise discussed herein, or where the host cells are those that naturally express the gene, as shown in these figures and which may be the native or a recombinant cell expressing the gene.

It is understood that “host cells” and “recombinant host cells” refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

The host cells expressing the polypeptides described herein, and particularly recombinant host cells, have a variety of uses. First, the cells are useful for producing kinase proteins or polypeptides that can be further purified to produce desired amounts of kinase protein or fragments. Thus, host cells containing expression vectors are useful for polypeptide production, as well as cells producing significant amounts of the polypeptide, for example, the high-expressors shown in FIGS. 2-14.

Host cells are also useful for conducting cell-based assays involving the kinase or kinase fragments. Thus, a recombinant host cell expressing a native kinase is useful to assay for compounds that stimulate or inhibit kinase function. This includes effector molecule or substrate binding, gene expression at the level of transcription or translation, effector protein, for example protein kinase, interaction, substrate interaction, interaction with ATP, GTP, AMP or GMP, and components of a signal transduction pathway.

Host cells are also useful for identifying kinase mutants in which these functions are affected. If the mutants naturally occur and give rise to a pathology, host cells containing the mutations are useful to assay compounds that have a desired effect on the mutant kinase (for example, stimulating or inhibiting function) which may not be indicated by their effect on the native kinase.

Recombinant host cells are also useful for expressing the chimeric polypeptides described herein to assess compounds that activate or suppress activation by means of a heterologous domain, segment, site, and the like, as disclosed herein.

Further, mutant kinases can be designed in which one or more of the various functions is engineered to be increased or decreased and used to augment or replace kinase proteins in an individual. Thus, host cells can provide a therapeutic benefit by replacing an aberrant kinase or providing an aberrant kinase that provides a therapeutic result. In one embodiment, the cells provide kinases that are abnormally active.

In another embodiment, the cells provide a kinase that is abnormally inactive. This kinase can compete with endogenous kinase in the individual.

In another embodiment, cells expressing kinases that cannot be activated are introduced into an individual in order to compete with endogenous kinase for substrate, ATP, GTP, or AMP, GMP, or effector molecule. For example, in the case in which excessive substrate is part of a treatment modality, it may be necessary to inactivate this molecule at a specific point in treatment. Providing cells that compete for the molecule , but which cannot be affected by kinase activation would be beneficial.

Homologously recombinant host cells can also be produced that allow the in situ alteration of endogenous kinase polynucleotide sequences in a host cell genome. The host cell includes, but is not limited to, a stable cell line, cell in vivo, or cloned microorganism. This technology is more filly described in WO 93/09222, WO 91/12650, WO 91/06667, U.S. Pat. No. 5,272,071, and U.S. 5,641,670. Briefly, specific polynucleotide sequences corresponding to the kinase polynucleotides or sequences proximal or distal to a kinase gene are allowed to integrate into a host cell genome by homologous recombination where expression of the gene can be affected. In one embodiment, regulatory sequences are introduced that either increase or decrease expression of an endogenous sequence. Accordingly, a kinase protein can be produced in a cell not normally producing it. Alternatively, increased expression of kinase protein can be effected in a cell normally producing the protein at a specific level. Further, expression can be decreased or eliminated by introducing a specific regulatory sequence. The regulatory sequence can be heterologous to the kinase protein sequence or can be a homologous sequence with a desired mutation that affects expression. Alternatively, the entire gene can be deleted. The regulatory sequence can be specific to the host cell or capable of functioning in more than one cell type. Still further, specific mutations can be introduced into any desired region of the gene to produce mutant kinase proteins. Such mutations could be introduced, for example, into the specific functional regions such as the cyclic nucleotide-binding site.

In one embodiment, the host cell can be a fertilized oocyte or embryonic stem cell that can be used to produce a transgenic animal containing the altered kinase gene. Alternatively, the host cell can be a stem cell or other early tissue precursor that gives rise to a specific subset of cells and can be used to produce transgenic tissues in an animal. See also Thomas et al., Cell 51:503 (1987) for a description of homologous recombination vectors. The vector is introduced into an embryonic stem cell line (e.g., by electroporation) and cells in which the introduced gene has homologously recombined with the endogenous kinase gene is selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos. WO 90/11354; WO 91/01140; and WO 93/04169.

The genetically engineered host cells can be used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a kinase protein and identifying and evaluating modulators of kinase protein activity.

Other examples of transgenic animals include non-human primates, sheep, dogs, cows, goats, chickens, and amphibians.

In one embodiment, a host cell is a fertilized oocyte or an embryonic stem cell into which kinase polynucleotide sequences have been introduced.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the kinase nucleotide sequences can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the kinase protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems, which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of S. cerevisiae (O'Gorman et al. (1991) Science 251:1351-1355. If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to a pseudopregnant female foster animal. The offspring born of this female foster animal will be a clone of the animal from which the cell, e.g., the somatic cell, is isolated.

Transgenic animals containing recombinant cells that express the polypeptides described herein are useful to conduct the assays described herein in an in vivo context. Accordingly, the various physiological factors that are present in vivo and that could affect substrate or effector binding, kinase activation, and signal transduction, may not be evident from in vitro cell-free or cell-based assays. Accordingly, it is useful to provide non-human transgenic animals to assay in vivo kinase function, including substrate interaction, the effect of specific mutant kinases on kinase function and substrate interaction, and the effect of chimeric kinases. It is also possible to assess the effect of null mutations, that is mutations that substantially or completely eliminate one or more kinase functions.

In general, methods for producing transgenic animals include introducing a nucleic acid sequence according to the present invention, the nucleic acid sequence capable of expressing the protein in a transgenic animal, into a cell in culture or in vivo. When introduced in vivo, the nucleic acid is introduced into an intact organism such that one or more cell types and, accordingly, one or more tissue types, express the nucleic acid encoding the protein. Alternatively, the nucleic acid can be introduced into virtually all cells in an organism by transfecting a cell in culture, such as an embryonic stem cell, as described herein for the production of transgenic animals, and this cell can be used to produce an entire transgenic organism. As described, in a further embodiment, the host cell can be a fertilized oocyte. Such cells are then allowed to develop in a female foster animal to produce the transgenic organism.

Vectors/Host Cells

The methods using the vectors and host cells discussed above are based on the vectors and host cells including, but not limited to, those described below.

The invention also provides methods using vectors containing the kinase polynucleotides. The term “vector” refers to a vehicle, preferably a nucleic acid molecule that can transport the kinase polynucleotides. When the vector is a nucleic acid molecule, the kinase polynucleotides are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of the kinase polynucleotides. Alternatively, the vector may integrate into the host cell genome and produce additional copies of the kinase polynucleotides when the host cell replicates.

The invention provides vectors for the maintenance (cloning vectors) or vectors for expression (expression vectors) of the kinase polynucleotides. The vectors can function in procaryotic or eukaryotic cells or in both (shuttle vectors).

Expression vectors contain cis-acting regulatory regions that are operably linked in the vector to the kinase polynucleotides such that transcription of the polynucleotides is allowed in a host cell. The polynucleotides can be introduced into the host cell with a separate polynucleotide capable of affecting transcription. Thus, the second polynucleotide may provide a trans-acting factor interacting with the cis-regulatory control region to allow transcription of the kinase polynucleotides from the vector. Alternatively, a trans-acting factor may be supplied by the host cell. Finally, a trans-acting factor can be produced from the vector itself.

It is understood, however, that in some embodiments, transcription and/or translation of the kinase polynucleotides can occur in a cell-free system.

The regulatory sequence to which the polynucleotides described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al. (1989) Molecular Cloning: A Laboratory Manual 2nd. ed., Cold Spring Harbor Laboratory Press, Cold Spring Harbor, N.Y.).

A variety of expression vectors can be used to express a kinase polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al (1989) Molecular Cloning: A Laboratory Manual 2nd. ed., Cold Spring Harbor Laboratory Press, Cold Spring Harbor, N.Y.

The regulatory sequence may provide constitutive expression in one or more host cells (i.e., tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The kinase polynucleotides can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate polynucleotide can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, and plant cells.

As described herein, it may be desirable to express the polypeptide as a fusion protein. Accordingly, the invention provides fusion vectors that allow for the production of the kinase polypeptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired polypeptide can ultimately be separated from the fusion moiety. Proteolytic enzymes include, but are not limited to, factor Xa, thrombin, and enterokinase. Typical fusion expression vectors include pGEX (Smith et al. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al. (1988) Gene 69:301-315) and pET 11d (Studier et al. (1990) Gene Expression Technology: Methods in Enzymology 185:60-89).

Recombinant protein expression can be maximized in a host bacteria by providing a genetic background wherein the host cell has an impaired capacity to proteolytically cleave the recombinant protein. (Gottesman, S. (1990) Gene Expression Technology: Methods in Enzymology 185, Academic Press, San Diego, Calif. 119-128). Alternatively, the sequence of the polynucleotide of interest can be altered to provide preferential codon usage for a specific host cell, for example E. coli. (Wada et al. (1992) Nucleic Acids Res. 20:2111-2118).

The kinase polynucleotides can also be expressed by expression vectors that are operative in yeast. Examples of vectors for expression in yeast e.g., S. cerevisiae include pYepSec1 (Baldari et al. (1987) EMBO J. 6:229-234 ), pMFa (Kurjan et al. (1982) Cell 30:933-943), pJRY88 (Schultz et al. (1987) Gene 54:113-123), and pYES2 (Invitrogen Corporation, San Diego, Calif.).

The kinase polynucleotides can also be expressed in insect cells using, for example, baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow et al. (1989) Virology 170:31-39).

In certain embodiments of the invention, the polynucleotides described herein are expressed in mammalian cells using mammalian expression vectors. Examples of mammalian expression vectors include pCDM8 (Seed, B. (1987) Nature 329:840) and pMT2PC (Kaufman et al. (1987) EMBO J 6:187-195).

It is further recognized that the nucleic acid sequences of the invention can be altered to contain codons, which are preferred, or non preferred, for a particular expression system. For example, the nucleic acid can be one in which at least one altered codon, and preferably at least 10%, or 20% of the codons have been altered such that the sequence is optimized for expression in E. coli, yeast, human, insect, or CHO cells. Methods for determining such codon usage are known in the art.

The expression vectors listed herein are provided by way of example only of the well-known vectors available to those of ordinary skill in the art that would be useful to express the kinase polynucleotides. The person of ordinary skill in the art would be aware of other vectors suitable for maintenance propagation or expression of the polynucleotides described herein. These are found for example in Sambrook et al. (1989) Molecular Cloning: A Laboratory Manual 2nd, ed., Cold Spring Harbor Laboratory, Cold Spring Harbor Laboratory Press, Cold Spring Harbor, N.Y.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the polynucleotide sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook et al. (Molecular Cloning: A Laboratory Manual, 2d ed., Cold Spring Harbor Laboratory, Cold Spring Harbor Laboratory Press, Cold Spring Harbor, N.Y).

Host cells can contain more than one vector. Thus, different nucleotide sequences can be introduced on different vectors of the same cell. Similarly, the kinase polynucleotides can be introduced either alone or with other polynucleotides that are not related to the kinase polynucleotides such as those providing trans-acting factors for expression vectors. When more than one vector is introduced into a cell, the vectors can be introduced independently, co-introduced or joined to the kinase polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the polynucleotides described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the polypeptide is desired, appropriate secretion signals are incorporated into the vector. The signal sequence can be endogenous to the kinase polypeptides or heterologous to these polypeptides.

Where the polypeptide is not secreted into the medium, the protein can be isolated from the host cell by standard disruption procedures, including freeze thaw, sonication, mechanical disruption, use of lysing agents and the like. The polypeptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the polypeptides described herein, the polypeptides can have various glycosylation patterns, depending upon the cell, or maybe non-glycosylated as when produced in bacteria. In addition, the polypeptides may include an initial modified methionine in some cases as a result of a host-mediated process.

Pharmaceutical Compositions

The invention encompasses use of the polypeptides, nucleic acids, and other agents in pharmaceutical compositions to administer to the cells in which expression of the phosophodiesterase is relevant and in disorders as disclosed herein. Uses are both diagnostic and therapeutic. The kinase nucleic acid molecules, protein, modulators of the protein, and antibodies (also referred to herein as “active compounds”) can be incorporated into pharmaceutical compositions suitable for administration to a subject, e.g., a human. Such compositions typically comprise the nucleic acid molecule, protein, modulator, or antibody and a pharmaceutically acceptable carrier. It is understood however, that administration can also be to cells in vitro as well as to in vivo model systems such as non-human transgenic animals.

The term “administer” is used in its broadest sense and includes any method of introducing the compositions of the present invention into a subject. This includes producing polypeptides or polynucleotides in vivo as by transcription or translation, in vivo, of polynucleotides that have been exogenously introduced into a subject. Thus, polypeptides or nucleic acids produced in the subject from the exogenous compositions are encompassed in the term “administer.”

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a kinase protein or anti-kinase antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents that modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 1 0,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Other Embodiments

In another aspect, the invention features, a method of analyzing a plurality of capture probes. The method can be used, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence; contacting the array with a 20893 preferably purified, nucleic acid, preferably purified, polypeptide, preferably purified, or antibody, and thereby evaluating the plurality of capture probes. Binding, e.g., in the case of a nucleic acid, hybridization with a capture probe at an address of the plurality, is detected, e.g., by signal generated from a label attached to the 20893 nucleic acid, polypeptide, or antibody.

The capture probes can be a set of nucleic acids from a selected sample, e.g., a sample of nucleic acids derived from a control or non-stimulated tissue or cell.

The method can include contacting the 20893 nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of 20893. Such methods can be used to diagnose a subject, e.g., to evaluate risk for a disease or disorder, to evaluate suitability of a selected treatment for a subject, to evaluate whether a subject has a disease or disorder. 20893 is associated with protein kinase activity, thus it is useful for disorders associated with abnormal angiogenesis such as cancer, diabetic blindness, age-related macular degeneration, rheumatoid arthritis, and psoriasis; liver fibrosis; and atherosclerosis.

The method can be used to detect SNPs.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express or mis express 20893 or from a cell or subject in which a 20893 mediated response has been elicited, e.g., by contact of the cell with 20893 nucleic acid or protein, or administration to the cell or subject 20893 nucleic acid or protein; contacting the array with one or more inquiry probe, wherein an inquiry probe can be a nucleic acid, polypeptide, or antibody (which is preferably other than 20893 nucleic acid, polypeptide, or antibody); providing a two dimensional array having a plurality of addresses, each address of the plurality being positionally distinguishable from each other address of the plurality, and each address of the plurality having a unique capture probe, e.g., wherein the capture probes are from a cell or subject which does not express 20893 (or does not express as highly as in the case of the 20893 positive plurality of capture probes) or from a cell or subject which in which a 20893 mediated response has not been elicited (or has been elicited to a lesser extent than in the first sample); contacting the array with one or more inquiry probes (which is preferably other than a 20893 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features, a method of analyzing 20893, e.g., analyzing structure, function, or relatedness to other nucleic acid or amino acid sequences. The method includes: providing a 20893 nucleic acid or amino acid sequence; comparing the 20893 sequence with one or more preferably a plurality of sequences from a collection of sequences, e.g., a nucleic acid or protein sequence database; to thereby analyze 20893.

Preferred databases include GenBank™. The method can include evaluating the sequence identity between a 20893 sequence and a database sequence. The method can be performed by accessing the database at a second site, e.g., over the internet.

In another aspect, the invention features, a set of oligonucleotides, useful, e.g., for identifying SNP's, or identifying specific alleles of 20893. The set includes a plurality of oligonucleotides, each of which has a different nucleotide at an interrogation position, e.g., an SNP or the site of a mutation. In a preferred embodiment, the oligonucleotides of the plurality are identical in sequence with one another (except for differences in length). The oligonucleotides can be provided with different labels, such that an oligonucleotide that hybridizes to one allele provides a signal that is distinguishable from an oligonucleotide that hybridizes to a second allele.

This invention is further illustrated by the following examples that should not be construed as limiting. The contents of all references, patents, and published patent applications cited throughout this application are incorporated herein by reference.

### EXPERIMENTAL

### EXAMPLE 1

### Identification and Characterization of human 20893 Protein Kinase

The human 20893 protein kinase sequence (FIG. 1), which is approximately 6828 nucleotides long including untranslated regions, contains a predicted methionine-initiated coding sequence of about 1986 nucleotides (nucleotides 1381-3366 of SEQ ID NO:1; SEQ ID NO:3). The coding sequence encodes a 661 amino acid protein (SEQ ID NO:2).

For general information regarding PFAM identifiers, PS prefix and PF prefix domain identification numbers, refer to Sonnhammer et al. (1997) Protein 28:405-420 and http//www.psc.edu/general/software/packages/pfam/pfam.html.

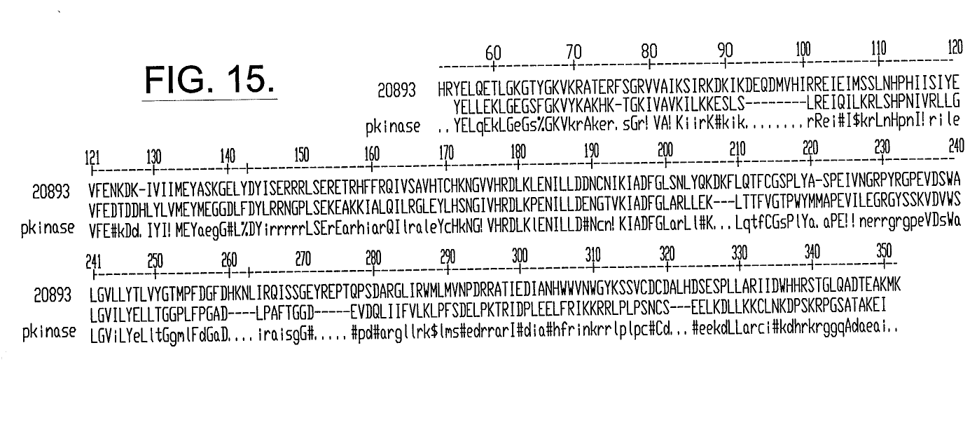

As used herein, the term “protein kinase domain” includes an amino acid sequence of about 200-400 amino acid residues in length and having a bit score for the alignment of the sequence to the protein kinase domain (HMM) of at least 8. Preferably, a protein kinase domain includes at least about 200-300 amino acids, more preferably about 250-300 amino acid residues, and has a bit score for the alignment of the sequence to the protein kinase domain (HMM) of at least 16 or greater. The protein kinase domain (HMM) has been assigned the PFAM Accession PF00069. An alignment of the protein kinase domain (amino acids 55 to 350 of SEQ ID NO:2) of human 20893 protein kinase with a consensus amino acid sequence derived from a hidden Markov model is depicted in FIG. 15.

In a preferred embodiment human 20893 protein kinase-like polypeptide or protein has a “protein kinase domain” or a region which includes at least about 200-400 more preferably about 200-300 or 250-300 amino acid residues and has at least about 60%, 70%, 80%, 90%, 95%, 99%, or 100% sequence identity with an “domain,” e.g., the protein kinase domain of human 20893 protein kinase-like polypeptides (e.g., amino acid residues 55 to 350 of SEQ ID NO:2).

To identify the presence of an “protein kinase” domain in a 20893-like protein sequence, and make the determination that a polypeptide or protein of interest has a particular profile, the amino acid sequence of the protein can be searched against a database of HMMs (e.g., the Pfam database, release 2.1) using the default parameters (http://www.sanger.ac.uk/Software/Pfarn/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference.

### EXAMPLE 2

### Tissue Distribution of 20893 mRNA

Expression levels of 20893 in various human tissue and cell types were determined by quantitative RT-PCR (Taqman® brand quantitative PCR kit, Applied Biosystems). The quantitative RT-PCR reactions were performed according to the kit manufacturer's instructions.

20893 was expressed in a variety of human tissue, including normal brain, kidney, heart, fibrotic liver, vascular endothelial cells and vascular smooth muscle cells. In normal liver cells, 20893 expression was lower than in activated hepatic stellate cells, fibrotic liver cells, and in BDL and CHCl₄ treated animals. The increased expression of 20893 in in vitro and in vivo models of liver fibrosis suggests an involvement of 20893 in liver fibrosis. 20893 was also upregulated during tube formation by endothelial cells on Matrigel and in a mouse ischemic hindlimb model of angiogenesis. The expression of 20893 in in vitro and in vivo models of angiogenesis suggests an involvement of 20893 in angiogenesis. Additionally, 20893 expression in the apoe mouse model of atherosclerosis was upregulated with time compared to the control, suggesting that 20893 is involved with atherosclerosis.

Northern blot hybridizations with various RNA samples are performed under standard conditions and washed under stringent conditions, i.e., 0.2×SSC at 65° C. A DNA probe corresponding to all or a portion of the 20893 cDNA (SEQ ID NO:1) can be used. The DNA is radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) are probed in ExpressHyb hybridization solution (Clontech) and washed at high stringency according to manufacturer's recommendations.

### EXAMPLE 3

### Recombinant Expression of 20893 in Bacterial Cells

In this example, 20893 is expressed as a recombinant glutathione-S-transferase (GST) fusion polypeptide in E. coli and the fusion polypeptide is isolated and characterized. Specifically, 20893 is fused to GST and this fusion polypeptide is expressed in E. coli, e.g., strain PEB199. Expression of the GST-20893 fusion protein in PEB199 is induced with IPTG. The recombinant fusion polypeptide is purified from crude bacterial lysates of the induced PEB 199 strain by affinity chromatography on glutathione beads. Using polyacrylamide gel electrophoretic analysis of the polypeptide purified from the bacterial lysates, the molecular weight of the resultant fusion polypeptide is determined.

### EXAMPLE 4

### Expression of Recombinant 20893 Protein in COS Cells

To express the 20893 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 20893 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 20893 DNA sequence is amplified by PCR using two primers. The 5′ primer contains the restriction site of interest followed by approximately twenty nucleotides of the 20893 coding sequence starting from the initiation codon; the 3′ end sequence contains complementary sequences to the other restriction site of interest, a translation stop codon, the HA tag or FLAG tag and the last 20 nucleotides of the 20893 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the 20893 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 20893-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 20893 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 20893 coding sequence is cloned directly into the polylinker of the pCDNA/Amp vector using the appropriate restriction sites. The resulting plasmid is transfected into COS cells in the manner described above, and the expression of the 20893 polypeptide is detected by radiolabelling and immunoprecipitation using a 20893 specific monoclonal antibody.

This invention may be embodied in many different forms and should not be construed as limited to the embodiments set forth herein; rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

What is claimed is:

1. A method of identifying an agent that binds a protein kinase, said method comprising combining an agent to be tested with a host cell expressing a nucleotide sequence selected from the group consisting of:
a) the nucleotide sequence corresponding to nucleotides 1381 to 3366 of SEQ ID NO:1;
b) the nucleotide sequence set forth in SEQ ID NO:3;
c) a nucleotide sequence encoding the amino acid sequence set forth in SEQ ID NO:2;
d) a nucleotide sequence corresponding to the cDNA insert of the plasmid deposited with ATCC as Patent Deposit No. PTA-2201;
e) a nucleotide sequence that is at least 60% identical to nucleotides 1381 to 3366 of SEQ ID NO:1;
f) a nucleotide sequence that is at least 60% identical to the nucleotide sequence set forth in SEQ ID NO:3; and,
g) a nucleotide sequence that is at least 60% identical to the nucleotide sequence corresponding to the cDNA insert of the plasmid deposited with ATCC as Patent Deposit No. PTA-2201; under conditions suitable for binding, and detecting the formation of a complex between said agent and said protein kinase; wherein said host cell is selected from the group consisting of brain, skeletal muscle, heart, fetal kidney; fetal heart; osteoblast; a virus-infected cell; vascular endothelium; vascular smooth muscle, and cells involved in tissue fibrosis.

2. The method of claiml, wherein said method is a competition assay, in which binding is determined in the presence of one or more agents.

3. A method of identifying a compound that inhibits binding of an agent to a protein kinase said method comprising combining a compound to be tested and said agent with a host cell expressing a nucleotide sequence selected from the group consisting of:
a) the nucleotide sequence corresponding to nucleotides 1381 to 3366 of SEQ ID NO:1;
b) the nucleotide sequence set forth in SEQ ID NO:3;
c) a nucleotide sequence encoding the amino acid sequence set forth in SEQ ID NO:2;
d) a nucleotide sequence corresponding to the cDNA insert of the plasmid deposited with ATCC as Patent Deposit No. PTA-2201;
e) a nucleotide sequence that is at least 60% identical to nucleotides 1381 to 3366 of SEQ ID NO: 1;
f) a nucleotide sequence that is at least 60% identical to the nucleotide sequence set forth in SEQ ID NO:3; and,
g) a nucleotide sequence that is at least 60% identical to the nucleotide sequence corresponding to the cDNA insert of the plasmid deposited with ATCC as Patent Deposit No. PTA-2201; under conditions suitable for binding of said agent thereto, and detecting the formation of a complex between said protein kinase and said agent, whereby inhibition of complex formation by said compound is indicative that said compound inhibits binding of said agent to said protein kinase; wherein said host cell is selected from the group consisting of brain, skeletal muscle, heart, fetal kidney; fetal heart; osteoblast; a virus-infected cell; vascular endothelium; vascular smooth muscle, and cells involved in tissue fibrosis.

4. The method of claim 3, wherein said compound is an antibody or antibody fragment.

5. A method of identifying an inhibitor of a protein kinase said method comprising combining an agent to be tested with a host cell expressing a nucleotide sequence selected from the group consisting of:
a) the nucleotide sequence corresponding to nucleotides 1381 to 3366 of SEQ ID NO:1;
b) the nucleotide sequence set forth in SEQ ID NO:3;
c) a nucleotide sequence encoding the amino acid sequence set forth in SEQ ID NO:2;
d) a nucleotide sequence corresponding to the cDNA insert of the plasmid deposited with ATCC as Patent Deposit No. PTA-2201;
e) a nucleotide sequence that is at least 60% identical to nucleotides 1381 to 3366 of SEQ ID NO:1;
f) a nucleotide sequence that is at least 60% identical to the nucleotide sequence set forth in SEQ ID NO:3; and,
g) a nucleotide sequence that is at least 60% identical to the nucleotide sequence corresponding to the cDNA insert of the plasmid deposited with ATCC as Patent Deposit No. PTA-220 1; under conditions suitable for detecting a protein kinase activity, and assessing the ability of said agent to inhibit said protein kinase activity, whereby inhibition of said protein kinase activity by said agent is indicative that said agent is an inhibitor; wherein said host cell is selected from the group consisting of brain, skeletal muscle, heart, fetal kidney; fetal heart; osteoblast; a virus-infected cell; vascular endothelium; vascular smooth muscle, and cells involved in tissue fibrosis.

6. The method of claim 5, wherein said protein kinase activity is a signaling activity or a cellular response.

7. An inhibitor of a protein kinase identified according to the method of claim 5, wherein said inhibitor is an antagonist.

8. A method for detecting the presence of a polypeptide in a sample, said method comprising contacting said sample with an agent that specifically allows detection of the presence of the polypeptide in the sample and then detecting the presence of the polypeptide, wherein said polypeptide is selected from the group consisting of:
a) a polypeptide having the amino acid sequence set forth in SEQ ID NO:2;
b) a polypeptide encoded by the nucleotide sequence corresponding to nucleotides 1381 to 3366 of SEQ ID NO:1;
c) a polypeptide encoded by the nucleotide sequence set forth in SEQ ID NO:3;
d) a polypeptide encoded by a nucleotide sequence corresponding to the cDNA insert of the plasmid deposited with ATCC as Patent Deposit No. PTA-2201;
e) a polypeptide encoded by a nucleotide sequence that is at least 60% identical to nucleotides 1381 to 3366 of SEQ ID NO:1;
f) a polypeptide encoded by a nucleotide sequence that is at least 60% identical to the nucleotide sequence set forth in SEQ ID NO:3;
g) a polypeptide encoded by a nucleotide sequence that is at least 60% identical to the nucleotide sequence corresponding to the cDNA insert of the plasmid deposited with ATCC as Patent Deposit No. PTA-2201; and,
h) a fragment of any of the polypeptides of a)-g) wherein said fragment comprises at least 15 contiguous amino acids of SEQ ID NO:2; wherein said sample is derived from a cell selected from the group consisting of brain, skeletal muscle, heart, fetal kidney, fetal heart, osteoblast, vascular endothelium, vascular smooth muscle, a virus-infected cell, and a cell involved in tissue fibrosis.

9. A method for modulating the level or activity of a polypeptide, the method comprising contacting said polypeptide with an agent under conditions that allow the agent to modulate the level or activity of the polypeptide, wherein said polypeptide is selected from the group consisting of:
a) a polypeptide having the amino acid sequence set forth in SEQ ID NO:2;
b) a polypeptide encoded by the nucleotide sequence corresponding to nucleotides 1381 to 3366 of SEQ ID NO:1;
c) a polypeptide encoded by the nucleotide sequence set forth in SEQ ID NO:3;
d) a polypeptide encoded by a nucleotide sequence corresponding to the cDNA insert of the plasmid deposited with ATCC as Patent Deposit No. PTA-2201;
e) a polypeptide encoded by a nucleotide sequence that is at least 60% identical to nucleotides 1381 to 3366 of SEQ ID NO:1;
f) a polypeptide encoded by a nucleotide sequence that is at least 60% identical to the nucleotide sequence set forth in SEQ ID NO:3;
g) a polypeptide encoded by a nucleotide sequence that is at least 60% identical to the nucleotide sequence corresponding to the cDNA insert of the plasmid deposited with ATCC as Patent Deposit No. PTA-2201; and,
h) a fragment of any of the polypeptides of a)-g) wherein said fragment comprises at least 15 contiguous amino acids of SEQ ID NO:2; wherein said modulation occurs in cells selected from the group consisting of brain, skeletal muscle, heart, fetal kidney, fetal heart, osteoblast, vascular endothelium, vascular smooth muscle, a virus-infected cell, and a cell involved in tissue fibrosis.

10. A method for detecting the presence of a nucleic acid molecule in a sample, said method comprising contacting said sample with an agent that specifically allows detection of the presence of the nucleic acid molecule in the sample and then detecting the presence of the nucleic acid molecule, the nucleic acid molecule having a nucleotide sequence selected from the group consisting of:
a) the nucleotide sequence corresponding to nucleotides 1381 to 3366 of SEQ ID NO:1;
b) the nucleotide sequence set forth in SEQ ID NO:3;
c) a nucleotide sequence encoding the amino acid sequence set forth in SEQ ID NO:2;
d) a nucleotide sequence corresponding to the cDNA insert of the plasmid deposited with ATCC as Patent Deposit No. PTA-2201;
e) a nucleotide sequence that is at least 60% identical to nucleotides 1381 to 3366 of SEQ ID NO:1;
f) a nucleotide sequence that is at least 60% identical to the nucleotide sequence set forth in SEQ ID NO:3; and,
g) a nucleotide sequence that is at least 60% identical to the nucleotide sequence corresponding to the cDNA insert of the plasmid deposited with ATCC as Patent Deposit No. PTA-2201; wherein said sample is derived from a cell selected from the group consisting of brain, skeletal muscle, heart, fetal kidney, fetal heart, osteoblast, vascular endothelium, vascular smooth muscle, a virus-infected cell, and a cell involved in tissue fibrosis.

11. The method of claim 10, wherein the method comprises contacting the sample with an oligonucleotide that hybridizes under stringent conditions to a nucleotide sequence selected from the group consisting of:
a) the nucleotide sequence corresponding to nucleotides 1381 to 3366 of SEQ ID NO:1;
b) the nucleotide sequence set forth in SEQ ID NO:3;
c) a nucleotide sequence encoding the amino acid sequence set forth in SEQ ID NO:2;
d) a nucleotide sequence corresponding to the cDNA insert of the plasmid deposited with ATCC as Patent Deposit No. PTA-2201;
e) a nucleotide sequence that is at least 60% identical to nucleotides 1381 to 3366 of SEQ ID NO: 1;
f) a nucleotide sequence that is at least 60% identical to the nucleotide sequence set forth in SEQ ID NO:3; and,
g) a nucleotide sequence that is at least 60% identical to the nucleotide sequence corresponding to the cDNA insert of the plasmid deposited with ATCC as Patent Deposit No. PTA-2201; and determining whether the oligonucleotide binds to the nucleic acid sequence in the sample.

12. The method of claim 11, wherein the nucleic acid whose presence is detected is mRNA.

13. A kit comprising reagents used for the method of claim 11, wherein the reagents comprise a compound that hybridizes under stringent conditions.

14. The method of claim 11 wherein a fragment of the nucleic acid is contacted.

15. A method for modulating the level or activity of a nucleic acid molecule, said method comprising contacting said nucleic acid molecule with an agent under conditions that allow the agent to modulate the level or activity of the nucleic acid molecule, said nucleic acid molecule having a nucleotide sequence selected from the group consisting of:
a) the nucleotide sequence corresponding to nucleotides 1381 to 3366 of SEQ ID NO:1;
b) the nucleotide sequence set forth in SEQ ID NO:3;
c) a nucleotide sequence encoding the amino acid sequence set forth in SEQ ID NO:2;
d) a nucleotide sequence corresponding to the cDNA insert of the plasmid deposited with ATCC as Patent Deposit No. PTA-2201;
e) a nucleotide sequence that is at least 60% identical to nucleotides 1381 to 3366 of SEQ ID NO:1;
f) a nucleotide sequence that is at least 60% identical to the nucleotide sequence set forth in SEQ ID NO:3; and,
g) a nucleotide sequence that is at least 60% identical to the nucleotide sequence corresponding to the cDNA insert of the plasmid deposited with ATCC as Patent Deposit No. PTA-2201; wherein said modulation is in a cell selected from the group consisting of brain, skeletal muscle, heart, fetal kidney, fetal heart, osteoblast, vascular endothelium, vascular smooth muscle, a virus-infected cell, and a cell involved in tissue fibrosis.

16. A method of modulating the activity of a polypeptide in a patient having a disorder selected from the group consisting of liver fibrosis, lung fibrosis, atherosclerosis, osteoporosis, osteopetrosis, cancer, diabetic blindness, psoriasis, age-related macular degeneration, viral infection, viral infection with hepatitis B virus, liver fibrosis resulting from hepatitis B virus infection, and disorders with abnormal angiogenesis, the method comprising administering to said patient a therapeutically effective amount of an agent that modulates the level or activity of a nucleotide sequence selected from the group consisting of:
a) the nucleotide sequence corresponding to nucleotides 1381 to 3366 of SEQ ID NO:1;
b) the nucleotide sequence set forth in SEQ ID NO:3;
c) a nucleotide sequence encoding the amino acid sequence set forth in SEQ ID NO:2;
d) a nucleotide sequence corresponding to the cDNA insert of the plasmid deposited with ATCC as Patent Deposit No. PTA-2201;
e) a nucleotide sequence that is at least 60% identical to nucleotides 1381 to 3366 of SEQ ID NO:1;
f) a nucleotide sequence that is at least 60% identical to the nucleotide sequence set forth in SEQ ID NO:3; and,
g) a nucleotide sequence that is at least 60% identical to the nucleotide sequence corresponding to the cDNA insert of the plasmid deposited with ATCC as Patent Deposit No. PTA-2201.

17. The method of claim 16 wherein said disorder is liver fibrosis.

18. A method of modulating the activity of a polypeptide in a patient having a disorder selected from the group consisting of liver fibrosis, lung fibrosis, atherosclerosis, osteoporosis, osteopetrosis, cancer, diabetic blindness, psoriasis, age-related macular degeneration, viral infection, viral infection with hepatitis B virus, liver fibrosis resulting from hepatitis B virus infection, and disorders with abnormal angiogenesis, the method comprising administering to a subject in need of treatment a therapeutically effective amount of an agent that modulates the level or activity of said polypeptide wherein said polypeptide is selected from the group consisting of:
a) a polypeptide having the amino acid sequence set forth in SEQ ID NO:2;
b) a polypeptide encoded by the nucleotide sequence corresponding to nucleotides 1381 to 3366 of SEQ ID NO:1;
c) a polypeptide encoded by the nucleotide sequence set forth in SEQ ID NO:3;
d) a polypeptide encoded by a nucleotide sequence corresponding to the cDNA insert of the plasmid deposited with ATCC as Patent Deposit No. PTA-2201;
e) a polypeptide encoded by a nucleotide sequence that is at least 60% identical to nucleotides 1381 to 3366 of SEQ ID NO:1;
f) a polypeptide encoded by a nucleotide sequence that is at least 60% identical to the nucleotide sequence set forth in SEQ ID NO:3;
g) a polypeptide encoded by a nucleotide sequence that is at least 60% identical to the nucleotide sequence corresponding to the cDNA insert of the plasmid deposited with ATCC as Patent Deposit No. PTA-2201.

19. A method for detecting a propensity of a patient to develop a liver disorder, said method comprising obtaining a sample from said patient and contacting said sample with an agent that specifically allows detection of the presence of a nucleic acid molecule in the sample and then detecting the presence of the nucleic acid molecule, the nucleic acid molecule selected from the group consisting of:
a) the nucleotide sequence corresponding to nucleotides 1381 to 3366 of SEQ ID NO:1;
b) the nucleotide sequence set forth in SEQ ID NO:3;
c) a nucleotide sequence encoding the amino acid sequence set forth in SEQ ID NO:2;
d) a nucleotide sequence corresponding to the cDNA insert of the plasmid deposited with ATCC as Patent Deposit No. PTA-2201;
e) a nucleotide sequence that is at least 60% identical to nucleotides 1381 to 3366 of SEQ ID NO:1;
f) a nucleotide sequence that is at least 60% identical to the nucleotide sequence set forth in SEQ ID NO:3; and,
g) a nucleotide sequence that is at least 60% identical to the nucleotide sequence corresponding to the cDNA insert of the plasmid deposited with ATCC as Patent Deposit No. PTA-2201; wherein said sample is derived from a patient with or at risk for liver disorders.


SEQUENCE LISTING (6 sequences)

SEQ ID NO: 1 - DNA, 6828 bp, H. sapiens
  CDS (1381)...(3366)
aatctcccaaggcctaaggaggcaagaggcctgcaaatcgcctcctgctcagcaaacgggttgctcagcaggcccggggtcctggtccaccccaggtccctggtttgcccacctccgatggcggccttcgctggcagggtgggcgcctctggggagccagctccgtcccggcgcctttagagccccatctcttccacgtccctggccttcctccccttccaggcggctgtccccgccggggtccagatggtgtcggagggccggcggttcgacggcgggcccggggttcagcctcccggcctccctccgtccctgactctcctttcttcggagagggcgcgggggccggggccaaagcgccgctcttggggttctcctggactcggagttgccccaggcgggcgcagctctgccccgcggggtgccagcctcgggcgggcaaggtccgtgagtcaccgcctgtaaccgaacaccaggcctccctgccccctcccccagctccggccgccaggctgcggcgacacctacaagaaaatgaaggggcgcccaggcccgcggcggccccggccgtatcgcgagcaggtcccggcggcccccggctcgcggcgctctttcttccccggccccggggctcggccagccgcaaccgccgccccggcgccagcaggaatccaggccgagcgaccggccccggagcccgaggcggcggagggcccgcggtagctgcgactggcgagcccgagagcgcccggggagggggcgcccggcttggaatttcccggtcccttccggcccagcgaggacaaagcactcctggccgccgccgccgccgccgccgtggcctacgccgcgccgcacaaagggcgagtcgcgacacgctcccatccccctcccagctcacggcggccccggccccgggtggctgcagggaggtgggggaagccctggctgcaccgcccctcgctccccctcccctggggccgcgcgagcgccgcccccgccccgtctgcgcgtcctcccggggaggggttggggggcgcggcgccccacataacactccccctcctgcgctgcgagccaccctctcccctccctcctgcaaacaccaccgcctcccctgccaccgccgccacctcgcccgacgctccacagctcgccgcggccggggggcggtgcgcggaccgtgcgcgccgcgggcgccagatgtgcagtccccgccgccgccagtgaccgagccgcagtccgagcggtatcgggccgcctccctgatgctgcgggggcgaccttgagcgtacagcggcttccctcggtggggaccccgacatcccagcgctgtgcccggtcttgccctctgtagcccggctcgccccgcgcttggacatggaaggggccgccgcgcctgtggcgggggaccgccccgacttggggctgggggcgccgggctctccccgagaggcggtggcgggggcgactgcagccctggagcccaggaagccgcacggggtgaagcggcatcaccacaagcacaacttgaagcaccgctacgagctgcaggagaccctgggcaaaggcacctacggcaaagtcaagcgggccaccgagaggttttctggccgagtggttgctataaaatccattcgtaaggacaaaattaaggatgaacaagacatggttcacatcagacgagagattgagatcatgtcatctctcaaccatcctcatatcatcagtatttatgaagtgtttgagaacaaagataagattgtgatcatcatggaatatgccagcaaaggggagctgtacgattacatcagtgagcggcgacgcctcagtgagagggagacccggcacttcttccggcagatcgtctctgctgtgcactattgtcacaagaacggtgtggtccaccgggacttgaagctggaaaatatactgctcgatgacaactgcaatattaagattgctgactttgggctttccaacctgtaccagaaggataagttcttacaaacgttttgtgggagtccactctatgcatctcctgagattgtcaatgggagaccttaccgagggccagaggtggacagctgggccctgggtgtgttgctttacactcttgtttatggaacaatgcccttcgatggtttcgatcacaaaaacctcattcggcaaatcagcagcggagagtaccgggagccaacacagccctcagatgctcgaggactcatacggtggatgctgatggtgaaccccgatcgccgggccactattgaggacattgccaaccactggtgggtgaactggggctataagagcagcgtgtgtgactgtgatgccctccatgactctgagtccccactcctggctcggatcattgactggcaccaccgttccacagggctgcaggctgacaccgaagccaaaatgaagggcctggccaaacccacgacctctgaggtcatgctagagcggcagcggtcgctgaagaaatccaagaaagagaatgactttgctcagtctggtcaggatgcagtgcctgaaagcccatccaagttgagttctaagaggcccaaggggatcctgaagaagcgaagcaacagcgagcatcgctctcacagcactggcttcattgaaggtgtagttggtcctgccttaccctctactttcaagatggagcaggacttgtgcaggactggcgtgctcctcccaagctcaccagaggcagaggtgccgggaaaactcagccccaagcagtcggccacgatgcccaagaaaggcatcttgaaaaagacccagcagagagaatcaggttactactcttccccagagcgcagtgagtcttcggagctgttggacagtaatgatgtgatgggcagcagcatcccctcccccagccccccggacccagccagggtaacctcccacagcctctcctgccggaggaagggcatcttgaaacacagcagcaaatactcagcgggcaccatggacccagccctggtcagccctgaaatgcccacactggaatccctgtcagagcctggtgtccctgccgagggcctctcccggagctacagccgcccttccagtgtcatcagcgatgacagcgtgctgtccagcgactcttttgacttgctggatttgcaggagaatcgccctgcccgccagcgcatccgcagctgcgtctctgcagaaaacttcctccagatccaggactttgaggggctccagaaccggccccggccccagtacctgaagcggtaccggaaccggctggcagacagcagcttctccctcctcacagacatggatgatgtgactcaggtctacaagcaagcgctggagatctgcagcaagctcaactagcattccagggcgcccaggggcgggcgggggtacgagggaggaaggggagcaagacttgggctcacaggctggttacctctttgctggctgtgacaacagactgaaaaaggattggcactgtctcacttggccaagtttgcagccttgagccaacacctaaaagggagaggtgggctcttctgccagttctgtcaattgtcagtcagaatttgggccctgtttggcatttgctttatggcacctcctagaggaccagctgtccaggggaggtggtattgaccggcactcagtgggtggagaggaagcatatgtggaaggagcatttccttagaaatgcttcattcatccagatgcttctggaggaggggcaggagacacttgggctgtttgccttgggcgagcccaaagaacttgccccttttctccttgcattagcaagctaggtctggctgcgtggagctggcaagtagatttcagcaacttgagcttgagttgatgatcaattaatagtggctgccagttgtgctggcgtaagtggcccacatcatggggaaggagtgctgtgattgactagtaatggctaccacgggaaagggaaggggaagcagtagcactaatgctatgtagttgtcatctttgatctggctaggccctgggaatcgggtttagtcatcctgtgggatctgtgttaactctttcatgccactggtgaggcatttgttaatttgctactcaactttgaggaaagacagggccttggtcagagagagaatgctctgaactctgctaaggacatagagtcagcccatggtgatttagctccttgctgttcacctcctctttcctgatgtctgccttgctctacagcacaacctcttgagggtggacagggagaaagatgatggtgtcagaggtcaaaactattatatatgacagggcacaagatggtctgtgatctttgcacagatgaatggaagttgatgcacaccaacaagaggcaacttgtcactttctttctcaatattaactggaatgctgcctcttgggttctcacctgcatggatgctttgagttggatgtgatactgtccatattctccagaggattacctggctgaaccattggctctgttcaccagtgacagatggtttccccatccactgagtgtagcatcctcagaggtaggcaagtttgcttctagggagttagcatgtagatgggatattgggatgaggaaaggaaaatcaggtagatggtgctttttttcccccaaatctaagtattctatgtcatggttttaaactttgccatgaactcctgggctttgggggaagagaaagttccattcatttaaatgaataaggtgttgaaagagtgcagggggttgggaggaagcatgtaagagagggaacatttccttagatgttacccagatggttctgggggagacagaaaagaggtgcggcaggactcttatcttaaaaagtaaacaaaacaaaacaaaacaaaacaaaaaaactagatatgtaatttctaaacacccagatcacaatgacaagatgccactccaaccatgggacaccttcatgatactaggtttgtacttcctggtctctgggatgacttcagattctgctggccaaggcaaattgaactcagttcaagatggccaccactggtagacgtgtagatagaaaagaggactggtcttgggaacatctttggaaaaaccaacaaacaatagttctagggagatgagaaaaaaattcaccttacagtgctaagaaagtgcattagaatggaattgccctttccttaaggagacagtttgggctctccccttgccaccggctctggtgttttggcttatgcgttccttcaggttgagctgagcagtgtgttatgggaagctgctcaatttcctttcattcaattccacctccttcctgaactctaatagaggttaaaagggaaaaaaaaaattctgtagatagcaaattgtgtgtgtgggggggggtgggggtgtgggtgcatggaggacaacctgcaactctgagctccctacttcctgcctcatttcatgcagtcttttctgaacagcctatgctgctgccctgctggccccttgtgcacggcagctggccgtgtccgtagctgtcagtatgacttagatctagctcctacctactggttgatgtgttttttccttttgccaagtgattgagtctgtttagtagtttccatcattctagtctttaagtaaaaatgacactattgaggaaagtcagtctactcccttcttcctccccccaaacacgtgttctcttttgtcaggaaactcagccagtgggctgtggcagagaaagtcctccactcagaggcagagactgagttaagtcataggtggccttaggcatctgcattgtttgcaggggttaagttttccttccagtgagggctggagggatgaattagctggtacctgaagccccgcttagctctgacactctgccaacatcctctgattctaggtgtggtgttgactgtcctttcaaggaaaaacttgcaatagagggaaaagccattaaagcagctccctgcttcatcattaagtcctgtcatccctaccagccaatcccagtcaaagaagttatgctttattcacttctgtggaattacaagtgagagacacttttaggacctgatggacaaagcaggagattcactgtcagctttcctggtcctctccttacttctgtgggccttgcaccgtcttagtttacacatctgccaaaggggtagaattacacttctttttacaggtaaatgtcaaggcacaatcagttttcaggaagtgcttcaagaccccaggtgaaatgaaaatgctaagtaccctctgaatggccatgcctgttaccaggtgctgcttcttcagatgatggggagcacttttcagggtgaaattcaggcgagttttgcccaggcctgctgtcttgagtacaaatgtgaatgatcgactgactgcttgttgccaaactggaaatgttctgtagggatttactggcatggtatcattcctagaagaaaaaaagagagaaacttgactgcacattaaaaaaaaaaaaatccacattgtgacttttatttaatttctattttttttggtaataaaaagttgacttttttatttgaatttgtcttttttatttattggtctgaaaggcatttcaaaggtattataataatatattggtgtaatttaattggtgcaacatgctttatggctcctgtcaaaattggttttcactcatttgattggtttgagcccagaacagcctacaggggaaaaacaagctggataaccacccaaagtgtttgtattttcgttggaaactgatttttgtttcattttggtttttgtttctgtttttatttttaaattaaataaattgcaatgaactg

SEQ ID NO: 2 - protein, 661 aa, H. sapiens
MEGAAAPVAGDRPDLGLGAPGSPREAVAGATAALEPRKPHGVKRHHHKHNLKHRYELQETLGKGTYGKVKRATERFSGRVVAIKSIRKDKIKDEQDMVHIRREIEIMSSLNHPHIISIYEVFENKDKIVIIMEYASKGELYDYISERRRLSERETRHFFRQIVSAVHYCHKNGVVHRDLKLENILLDDNCNIKIADFGLSNLYQKDKFLQTFCGSPLYASPEIVNGRPYRGPEVDSWALGVLLYTLVYGTMPFDGFDHKNLIRQISSGEYREPTQPSDARGLIRWMLMVNPDRRATIEDIANHWWVNWGYKSSVCDCDALHDSESPLLARIIDWHHRSTGLQADTEAKMKGLAKPTTSEVMLERQRSLKKSKKENDFAQSGQDAVPESPSKLSSKRPKGILKKRSNSEHRSHSTGFIEGVVGPALPSTFKMEQDLCRTGVLLPSSPEAEVPGKLSPKQSATMPKKGILKKTQQRESGYYSSPERSESSELLDSNDVMGSSIPSPSPPDPARVTSHSLSCRRKGILKHSSKYSAGTMDPALVSPEMPTLESLSEPGVPAEGLSRSYSRPSSVISDDSVLSSDSFDLLDLQENRPARQRIRSCVSAENFLQIQDFEGLQNRPRPQYLKRYRNRLADSSFSLLTDMDDVTQVYKQALEICSKLN

SEQ ID NO: 3 - DNA, 1986 bp, H. sapiens
atggaaggggccgccgcgcctgtggcgggggaccgccccgacttggggctgggggcgccgggctctccccgagaggcggtggcgggggcgactgcagccctggagcccaggaagccgcacggggtgaagcggcatcaccacaagcacaacttgaagcaccgctacgagctgcaggagaccctgggcaaaggcacctacggcaaagtcaagcgggccaccgagaggttttctggccgagtggttgctataaaatccattcgtaaggacaaaattaaggatgaacaagacatggttcacatcagacgagagattgagatcatgtcatctctcaaccatcctcatatcatcagtatttatgaagtgtttgagaacaaagataagattgtgatcatcatggaatatgccagcaaaggggagctgtacgattacatcagtgagcggcgacgcctcagtgagagggagacccggcacttcttccggcagatcgtctctgctgtgcactattgtcacaagaacggtgtggtccaccgggacttgaagctggaaaatatactgctcgatgacaactgcaatattaagattgctgactttgggctttccaacctgtaccagaaggataagttcttacaaacgttttgtgggagtccactctatgcatctcctgagattgtcaatgggagaccttaccgagggccagaggtggacagctgggccctgggtgtgttgctttacactcttgtttatggaacaatgcccttcgatggtttcgatcacaaaaacctcattcggcaaatcagcagcggagagtaccgggagccaacacagccctcagatgctcgaggactcatacggtggatgctgatggtgaaccccgatcgccgggccactattgaggacattgccaaccactggtgggtgaactggggctataagagcagcgtgtgtgactgtgatgccctccatgactctgagtccccactcctggctcggatcattgactggcaccaccgttccacagggctgcaggctgacaccgaagccaaaatgaagggcctggccaaacccacgacctctgaggtcatgctagagcggcagcggtcgctgaagaaatccaagaaagagaatgactttgctcagtctggtcaggatgcagtgcctgaaagcccatccaagttgagttctaagaggcccaaggggatcctgaagaagcgaagcaacagcgagcatcgctctcacagcactggcttcattgaaggtgtagttggtcctgccttaccctctactttcaagatggagcaggacttgtgcaggactggcgtgctcctcccaagctcaccagaggcagaggtgccgggaaaactcagccccaagcagtcggccacgatgcccaagaaaggcatcttgaaaaagacccagcagagagaatcaggttactactcttccccagagcgcagtgagtcttcggagctgttggacagtaatgatgtgatgggcagcagcatcccctcccccagccccccggacccagccagggtaacctcccacagcctctcctgccggaggaagggcatcttgaaacacagcagcaaatactcagcgggcaccatggacccagccctggtcagccctgaaatgcccacactggaatccctgtcagagcctggtgtccctgccgagggcctctcccggagctacagccgcccttccagtgtcatcagcgatgacagcgtgctgtccagcgactcttttgacttgctggatttgcaggagaatcgccctgcccgccagcgcatccgcagctgcgtctctgcagaaaacttcctccagatccaggactttgaggggctccagaaccggccccggccccagtacctgaagcggtaccggaaccggctggcagacagcagcttctccctcctcacagacatggatgatgtgactcaggtctacaagcaagcgctggagatctgcagcaagctcaactag

SEQ ID NO: 4 - protein, 272 aa, Artificial Sequence
  This is a consensus protein kinase domain.
YELLEKLGEGSFGKVYKAKHKTGKIVAVKILKKESLSLREIQILKRLSHPNIVRLLGVFEDTDDHLYLVMEYMEGGDLFDYLRRNGPLSEKEAKKIALQILRGLEYLHSNGIVHRDLKPENILLDENGTVKIADFGLARLLEKLTTFVGTPWYMMAPEVILEGRGYSSKVDVWSLGVILYELLTGGPLFPGADLPAFTGGDEVDQLIIFVLKLPFSDELPKTRIDPLEELFRIKKRRLPLPSNCSEELKDLLKKCLNKDPSKRPGSATAKEI

SEQ ID NO: 5 - DNA, 2884 bp, H. sapiens
  CDS (24)...(2009)
cccggctcgccccgcgcttggacatggaaggggccgccgcgcctgtggcgggggaccgccccgacttggggctgggggcgccgggctctccccgagaggcggtggcgggggcgactgcagccctggagcccaggaagccgcacggggtgaagcggcatcaccacaagcacaacttgaagcaccgctacgagctgcaggagaccctgggcaaaggcacctacggcaaagtcaagcgggccaccgagaggttttctggccgagtggttgctataaaatccattcgtaaggacaaaattaaggatgaacaagacatggttcacatcagacgagagattgagatcatgtcatctctcaaccatcctcatatcatcagtatttatgaagtgtttgagaacaaagataagattgtgatcatcatggaatatgccagcaaaggggagctgtacgattacatcagtgagcggcgacgcctcagtgagagggagacccggcacttcttccggcagatcgtctctgctgtgcactattgtcacaagaacggtgtggtccaccgggacttgaagctggaaaatatactgctcgatgacaactgcaatattaagattgctgactttgggctttccaacctgtaccagaaggataagttcttacaaacgttttgtgggagtccactctatgcatctcctgagattgtcaatgggagaccttaccgagggccagaggtggacagctgggccctgggtgtgttgctttacactcttgtttatggaacaatgcccttcgatggtttcgatcacaaaaacctcattcggcaaatcagcagcggagagtaccgggagccaacacagccctcagatgctcgaggactcatacggtggatgctgatggtgaaccccgatcgccgggccactattgaggacattgccaaccactggtgggtgaactggggctataagagcagcgtgtgtgactgtgatgccctccatgactctgagtccccactcctggctcggatcattgactggcaccaccgttccacagggctgcaggctgacaccgaagccaaaatgaagggcctggccaaacccacgacctctgaggtcatgctagagcggcagcggtcgctgaagaaatccaagaaagagaatgactttgctcagtctggtcaggatgcagtgcctgaaagcccatccaagttgagttctaagaggcccaaggggatcctgaagaagcgaagcaacagcgagcatcgctctcacagcactggcttcattgaaggtgtagttggtcctgccttaccctctactttcaagatggagcaggacttgtgcaggactggcgtgctcctcccaagctcaccagaggcagaggtgccgggaaaactcagccccaagcagtcggccacgatgcccaagaaaggcatcttgaaaaagacccagcagagagaatcaggttactactcttccccagagcgcagtgagtcttcggagctgttggacagtaatgatgtgatgggcagcagcatcccctcccccagccccccggacccagccagggtaacctcccacagcctctcctgccggaggaagggcatcttgaaacacagcagcaaatactcagcgggcaccatggacccagccctggtcagccctgaaatgcccacactggaatccctgtcagagcctggtgtccctgccgagggcctctcccggagctacagccgcccttccagtgtcatcagcgatgacagcgtgctgtccagcgactcttttgacttgctggatttgcaggagaatcgccctgcccgccagcgcatccgcagctgcgtctctgcagaaaacttcctccagatccaggactttgaggggctccagaaccggccccggccccagtacctgaagcggtaccggaaccggctggcagacagcagcttctccctcctcacagacatggatgatgtgactcaggtctacaagcaagcgctggagatctgcagcaagctcaactagcattccagggcgcccaggggcgggcgggggtacgagggaggaaggggagcaagacttgggctcacaggctggttacctctttgctggctgtgacaacagactgaaaaaggattggcactgtctcacttggccaagtttgcagccttgagccaacacctaaaagggagaggtgggctcttctgccagttctgtcaattgtcagtcagaatttgggccctgtttggcatttgctttatggcacctcctagaggaccagctgtccaggggaggtggtattgaccggcactcagtgggtggagaggaagcatatgtggaaggagcatttccttagaaatgcttcattcatccagatgcttctggaggaggggcaggagacacttgggctgtttgccttgggcgagcccaaagaacttgccccttttctccttgcattagcaagctaggtctggctgcgtggagctggcaagtagatttcagcaacttgagcttgagttgatgatcaattaatagtggctgccagttgtgctggcgtaagtggcccacatcatggggaaggagtgctgtgattgactagtaatggctaccacgggaaagggaaggggaagcagtagcactaatgctatgtagttgtcatctttgatctggctaggccctgggaatcgggtttagtcatcctgtgggatctgtgttaactctttcatgccactggtgaggcatttgttaatttgctactcaactttgaggaaagacagggccttggtcagagagagaatgctctgaactctgctaaggacatagagtcagcccatggtgatttagctccttgctgttcacctcctctttcctgatgtctgccttgctctacagcacaacct

SEQ ID NO: 6 - protein, 661 aa, H. sapiens; identical to SEQ ID NO: 2